(19) United States
(12) Patent Application Publication
(10) Pub. No.: US 2007/0299458 A1
(43) Pub. Date: Dec. 27, 2007

(54) Renewable rotary skin lancet

(76) Inventor: John A. Epple, Kansas City, MO (US)

Correspondence Address: John Albert Epple Jr., 309 West 124th Street, Kansas City, MO, 64145, US

(21) Appl. No.: 11/810,955

(22) Filed: Jun. 7, 2007

Related U.S. Application Data
(60) Provisional application No. 60/812,202, filed on Jun. 10, 2006.
(60) Provisional application No. 60/873,411, filed on Dec. 6, 2006.

(51) Int. Cl. A61B 17/32

(52) U.S. Cl. 606/181

(57) ABSTRACT

A lancet device used as a puncturing tool for creating a hole in a person's skin to obtain blood for testing. The device comprises a hammer which propels needle points into skin to release a droplet of blood. The device comprises a method for adjusting the needle's penetration. The device comprises a renewable wheel to carry the needles, and it is exchanged for a new needle wheel when no unused needles remain. The device comprises a method for rotating the needle wheel. The device shields the needle points from human touch. The device comprises a numbering system to indicate the needles remaining unused. The device is designed to minimize pain and bruising. Its simplicity means even children, the aged, and the handicapped can safely and economically use this device.

I am hereby claiming whatever benefits accrue to the present invention and its application from 2 (two) provisional patent applications filed earlier by the present inventor at the USPTO offices. The first provisional patent application, “Renewable Rotary Skin Lancet”, was filed Jun. 10, 2006, and was granted an application No. 60/812,202. The second provisional patent application, “Shouldered Lancet Needle Carriers”, was filed Dec. 6, 2006, and was granted an application No. 60/873,411.

The present invention is a tool used to perform the task of lancing a person's skin. The commonly-applied term used for such tools is “Lancet”. Thus the name “Lancet” is used. Because the target of the Lancet is “Skin”, that terminology is included. The present invention includes as one of its components a “Rotary” wheel, so that name is included. The wheel is made so it can be replaced and is in that way “Renewable, so that name is included. The title “Renewable Rotary Skin Lancet” was chosen to adequately describe the type and function of the present invention.

### CROSS REFERENCES TO RELATED APPLICATIONS

U.S. Patent Documents

| | | | |
|---|---|---|---|
| 7,175,641 B1 | Feb. 13, 2007 | Schraga | 606/181 |
| 7,105,006 B2 | Sep. 12, 2006 | Schraga | 606/182 |
| 7,087,068 B1 | Aug. 8, 2006 | Marshall, et al. | 606/182 |
| 6,949,111 B2 | Sep. 27, 2005 | Schraga | 606/182 |
| 6,887,253 B2 | May 3, 2005 | Schraga | 606/181 |
| 6,660,018 B2 | Dec. 9, 2003 | Lum, et al. | 606/181 |
| 6,228,100 1 | May 8, 2001 | Schraga | 606/183 |
| 5,645,555 | Jul. 8, 1997 | Davis, et al. | 606/182 |
| 5,476,474 | Dec. 19, 1995 | Davis, et al. | 606/181 |
| 4,794,926 | Jan. 3, 1989 | Munsch, et al. | 128/314 |
| 4,157,086 | Jun. 5, 1979 | Maiorano, et al. | 128/637 |

### COMPARISON BETWEEN THE REFERENCED PATENTS AND THE RENEWABLE ROTARY SKIN LANCET

U.S. Pat. No. 7,175,641 B1 is for a single-needle type lancet, whereas the present patent application is for a multi-needle lancet. Because of this there is a distinct difference between the two lancet devices.

U.S. Pat. No. 7,105,006 B2 for is a single-needle lancet, whereas the present patent application is for a multi-needle lancet. Because of this there is a distinct difference between the two lancet devices.

U.S. Pat. No. 7,087,068 B1 is for a single-needle lancet, whereas the present patent application is for a multi-needle lancet. Because of this there is a distinct difference between the two lancet devices.

U.S. Pat. No. 6,949,111 B2 is for a single-needle lancet, whereas the present patent application is for a multi-needle lancet. Because of this there is a distinct difference between the two lancet devices.

U.S. Pat. No. 6,887,253 B2 is for the needle-depth, adjustment-mechanism for a single-needle lancet, whereas the present patent application is for a multi-needle lancet, which, although it too embodies a needle-depth adjustment, makes use of an entirely different mechanical process to revise the needle depth. Because of this there is a distinct difference between the two lancet devices.

U.S. Pat. No. 6,660,018 B2 is for a multi-needle type lancet, as is the present patent application. However, U.S. Pat. No. 6,660,018 B2 embodies several needles, each parallel to the others, and all of which effectuate a different location of skin puncture at essentially the same moment within close distances from each other, and the device may be repeatedly used, possibly leading to unwanted infections when used on different subjects or at different times. In comparison, the present patent application is for a multi-needle lancet arranged in a non-parallel, radial fashion, each of which can be used only alone, and only one time, limiting the possibility of unwanted infections. Because of these facts, there is a distinct difference between the two lancets.

U.S. Pat. No. 6,228,100 1 is for a device which has some similarities to the device in the present patent application. The similarities are these. Both have a multitude of needle carriers arrayed in a radial fashion from a common center. However, the needle carriers are distinctly different. Both have a mechanism for driving the needle carriers radially into the target's skin, but the mechanisms for doing this are distinctly different. Both have a mechanism for rotating a needle-carrying wheel, but the mechanisms for doing this are distinctly different.

There are other distinct differences. Some of them are listed following.

The ratcheting parts of the device of U.S. Pat. No. 6,228,100 1 are unchangeably clustered in a circle close to the axial center of said device, and this means that only a very-limited, unchangeable number of ratcheting points can be built into a device constructed under the limitations of the design of U.S. Pat. No. 6,228,100 1. In said design there must be a ratcheting point accompanying each needle in said device, and the restriction on numbers created by the ratcheting-number restriction means that only a comparatively few needles can be built into that patented device. In comparison, the present patent application will embody many ratcheting points, actually named Cogs in the present invention, by which to advance each needle in its turn, and, being near the outer periphery of the needle-bearing wheel of said present device, comparatively, many more needles can be made available for use in a device of practical size in the device of the present patent application. In short, the greater the diameter of the Cog Wheels and Needle-Carrier Wheels in the present patent application, the more Needle Carriers will be available in it, whereas, in the device shown for U.S. Pat. No. 6,228,100 1 only the same, limited, maximum number of Needles will ever be available, no matter how large the device is constructed. One of the primary objectives for the device in the present patent application was having a multitude of Needles available in it, and in the present invention the number available is limited only by the device needing to be built of a practicable physical size. Although the number of needles available in the present invention can be from sixty count to 100 count without seeming to be overly large, the optimal number will not be known until prototype units of the present invention can be made and tested. In comparison, the number of needles fixedly available in the device in U.S. Pat. No. 6,228,100 1 seems to be six needles as only six ratcheting points are shown on the drawings, this irrespective of the fact the drawings show fifteen needles, an impossible number if the drawings are correct with respect to the ratcheting points.

Further, U.S. Pat. No. 6,228,100 1 is for a multi-needle type lancet device in which the location of the puncturing needle in a finger-shaped indentation makes it impractical to attempt a puncture on a flat segment of skin such as is the target for blood samples used by some persons who have an aversion to injuring their fingers, and who want to use their arms or thighs for obtaining blood samples. Notable examples of that sort of person might be musicians who play stringed instruments, or shop clerks whose hands are prominent in their work. The soreness and blue bruising evidenced by persons who stick their fingers several times daily cannot be masked, and in some persons they can be hurtful and ugly. The needle points of the device of U.S. Pat. No. 6,228,100 1 might be unable to project as far as the location of the skin at an arm or thigh, and even if they could, the depth of penetration would seem to be ungovernable. In comparison, the device in the present patent application projects the needle point outside the bounds of the device case, making it usable on fingers, thighs, arms, or the skin on nearly any part of a human body.

Further, U.S. Pat. No. 6,228,100 1 has no mechanism for adjusting the distance its needle points project into the skin when blood samples are being obtained. This is unfortunate due to the fact that on different persons the skin thickness can vary considerably. For example, a carpenter who has practiced the carpentry trade for many years may have thick, calloused skin on his or her fingers due to the years of contact with rough objects and the beating the fingers would have taken from missing the nail and hitting a finger instead. Clerks in a linen store would most likely have thin skin on the fingers because their work would not likely have caused damage to their finger's skin. Older people sometimes sustain a thinning of the skin all over their body as they age. Children will mostly have very thin and very sensitive skin. Children will have a natural dread of having any sort of needle penetrating their skin, and may more strenuously object when a needle is used which either penetrates too deeply than is necessary to obtain a sufficiently large blood sample, or even worse, insufficiently deep to obtain any blood at all, thereby necessitating multiple punctures while seeking the needed amount of blood. The inventor of the device being presently applied for is a diabetic who currently must use a lancet device for obtaining blood samples four times each day, and one of the primary considerations of said inventor in designing the presently-applied-for Renewable Rotary Skin Lancet was to provide an effective means of adjusting the depth of needle penetration. The present patent application embodies such a mechanism. Said inventor knows of no other lancet device having a similar adjusting mechanism.

Further, the device described in U.S. Pat. No. 6,228,100 1 does not appear to embody an easily-removable disc, wheel, or cartridge in which the needle-containing objects are located. It appears that the individual needle-carrying parts must be manually removed and replaced one at a time if multiple uses of the said device's needle spaces are to exist. Replacing the needles one by one would entail several objectionable processes. The opportunity for the needles, exposed as they would be, to become septic, is objectionable. The chance of inadvertent finger sticks as the needle devices are either being removed or replaced, and the attendant chance of unwanted infections, is absolutely undesirable in the present day of scourges such as the AIDS disease.

Further, there will be many users of a lancet device who would be incapable of safely removing used lancets one at a time and then replacing them. It is true that the device described in U.S. Pat. No. 6,228,100 1 might be able to be used until its supply of needles is exhausted, but it should then be discarded in entirety to maintain the safety which it should maintain, and doing so would make the use if this device overly expensive compared to many other types of lancet devices. In comparison, the present patent application uses an easily-removable wheel for containing the Needle Carriers and Needles, and it can be easily replaced by a factory-made, replacement needle-wheel manufactured and assembled in a sterile environment.

Further, the present patent application's needles are at all times, except at the moment of skin puncture, shielded from the possibility of contamination from touching, or from infections arising from an accidental finger sticking during human contact with the used or replacement needle-bearing wheel. And, once used, its needle-bearing wheel can be thrown away without using a sharps container, and without creating an undue hazard. This, of course, results in a comparatively economical use of the device of the present application. The device shown in U.S. Pat. No. 6,228,100 1 seems to leave a used needle point exposed until a new needle is rotated into the indented edge space. This would mean that accidental, unwanted, possibly hazardous, punctures could take place on that device. The needles used in this type of device are so small as to be insignificantly visible, and a person desiring to use the device could unknowingly easily place a finger in the indentation before it has been newly cocked, and that might be very dangerous.

Further, the process of replacing the needle-bearing wheel in the device of the present application is simple enough to safely perform that only very young children and physically or mentally incapable persons would need to be excluded from the personal use of the device.

Further, the device of U.S. Pat. No. 6,228,100 1 features a protective covering over the point of its needles. These covers must be removed one by one before the needles are used for punctures. U.S. Pat. No. 6,228,100 1 makes use of an included, physically-separate device for removing the protective covers. This process would be a burden to anyone using said device, particularly young or elderly users, or anyone else having limited physical or mental capacities. In comparison, the present patent application does not require needle point coverings to maintain safety in its use, because the needle points in said present application are hidden away from human contact until the time they are used momentarily to make a skin puncture, and they are then immediately retracted from exposure, so there is no need for a separate device to prepare the needles for use. Needing to have available an altogether separate device in order to make use of the primary lancet device is likely to be troublesome. The device of the present application has no such problem connected to its use.

Further, The enclosing case of the device of U.S. Pat. No. 6,228,100 1 is a bulky unit, with protruding parts. This will make it inconvenient to grasp in the hand of the user, and bulky for carrying in a pocket or purse. The enclosing case of the device in the present application is a flat shape, and, when it is constructed in a practicable size, is able to be easily grasped in the user's hand, and able to be fitted easily in a pocket or purse.

Further, because the needles used in a desirable lancet must be made with a very small diameter in order to be as comfortable to use as is possible, when the skin of a person is punctured in order to obtain a droplet of blood for testing, quite often the flow of blood from the puncture wound must be encouraged to appear by squeezing the skin around the puncture soon after it is made. The device in the present application embodies a raised rim around the hole thru which the device's needles project at the moment they are making a puncture. This rim, when pressed against the target skin, will leave a briefly-visible indentation, showing exactly where the puncture has occurred. If the blood does not quickly appear, the spot which must be squeezed to start it flowing is easily determined from said indentation In comparison, the device in U.S. Pat. No. 6,228,100 1 has no such raised rim, and, as a result the exact point of puncture may be indeterminable when blood does not immediately appear. The result may be that an undesirable second, or even third, puncture may be required to release blood, and an undesirable deeper penetration of the puncturing needle on the patented device may ordinarily be necessary for effective use of that patented device.

Whereas the device in the present application makes use of a trigger button which moves a sort of holed gate away from the striking end of the Hammer, and this releases the Hammer so the Needle Carrier and Needle will be impelled toward the target skin, the device in U.S. Pat. No. 6,228,100 1 employs an altogether different sort of releasing mechanism.

In the device of the present application there is a choice of two different types of systems used to advance the needle-bearing wheel one needle space forward after using the prior needle for a puncture. Two systems are included so that the manufacturer may use whichever one will be most easily produced and/or most effective in its use. The needle-advancement process used in the device in U.S. Pat. No. 6,228,100 1 makes use of an entirely dissimilar mechanism.

In the device of the present application there is a detent device employed to accurately center the projecting needle in the aperture in the case containing said device so that there will be no interference between the case and the needle when the needle is projected. The device in U.S. Pat. No. 6,228,100 1 has no such detent centering mechanism.

There are several needle carrier designs shown in the drawings made for the device of the present application. Four of these embody devices in the needle carrier and/or the needle carrier's wheel whereby accidental reuse of a used needle in forestalled. And, with the use of one of those four needles this is true even if the used needle carrier wheel is accidentally reinserted for use. It is impossible, given the scant information provided for the needle devices in U.S. Pat. No. 6,228,100 1 to determine whether its needle devices embody any means to disallow accidental reuse of its used needles. In any event, that patent's text and drawings do not seem to specify such means.

Any commercially-sold lancet device will be used by a wide range of people. Diabetics, for example, use several lancets daily for determining the glucose content of their blood, and their insulin intake is then based on the blood tests. Diabetes is prevalent in people all ages, all genders, all races, and all ages, and it is reportedly becoming more prevalent. Diabetes is probably the largest reason for a person to have regular blood tests requiring obtaining a drop or two of blood, and sufferers of this disease must regularly test their blood. Modern lancet devices are the best way to obtain the blood for testing. But it is almost mandatory that the lancet devices used can be used with uncomplicated methods. As regards the device in U.S. Pat. No. 6,228,100 1, it is overly complicated to use, so much so that it is likely that its use is too complicated for many people requiring a lancet's use. On the other hand the device in the present application can be successfully and safely used by a young child, a blind person, or even a one-armed person, so long as there is present in them a moderate amount of intelligence and physical ability. The only operations required in using the device in the present application are opening the case, loading a needle-carrier wheel, cocking the hammer, pressing the firing button, and when the needle supply is exhausted, again opening the case, and replacing the used needle carrier wheel. All these are easily performed, making its use practical for a wide range of people.

U.S. Pat. No. 5,645,555 carries the word “Rotary” in its name, and, in fact, the part used to penetrate a patient's skin does rotate within the device in the process of performing its intended function. However, it differs from the device contemplated in the present patent application in the following ways. The device in U.S. Pat. No. 5,645,555 embodies a knife-type edge to effectuate entrance into a skin surface in a cutting fashion. The present patent application embodies a group of needles which effectuate entrance into a skin surface in a puncturing fashion which is much less intrusive and much less traumatic than the imposition of a cut in the skin. Further, the device in U.S. Pat. No. 5,645,555 embodies only one cutting part which is able to be repeatedly used. This fact means it can lead to unwanted infections when used on different subjects or at different times. The present patent application embodies a group of needles each of which can be used but once, thereby limiting the possibility of infections.

U.S. Pat. No. 5,476,474 is simply an earlier embodiment of many of the same features as U.S. Pat. No. 5,645,555, and its differences from the device contemplated in the present patent application are essentially the same.

U.S. Pat. No. 4,794,926 is in fact a lancet device having a rotating disc with fingers. Each finger has a single tooth which is used to puncture a person's skin for the purpose of obtaining a sample of blood. U.S. Pat. No. 4,794,926 differs from the device in the present application in these ways. U.S. Pat. No. 4,794,926 uses a sharpened tooth. The present application uses needles. The tooth used by the device in U.S. Pat. No. 4,794,926 will cause a larger hole in skin than a needle, and this will cause greater trauma and more emission of blood than may be desirable. The tooth used by the device in U.S. Pat. No. 4,794,926 projects from the case of its device perpendicularly to the plane of the case body. In the device of the present application the needle projects from a very small opening and in the same plane as the body of its case, and the positioning of the puncture being made can more exactly be selected because more of the skin area will be visible when the device in the present application is used since less of the device case blocks a view of the skin area. It appears that the device in U.S. Pat. No. 4,794,926, as constituted, will permit reuse of its inserted toothed disc more than one time, possibly leading to unwanted infections. In the most preferred embodiments of the Needle Carriers used in the present invention the Needle Carriers are mechanically blocked from being casually or accidentally reused, even if the used Needle Carrier Wheel is accidentally reinserted, and, thereby, the opportunity for unwanted infections is reduced.

U.S. Pat. No. 4,157,086 embodies a knife-type edge to effectuate entrance into a skin surface in a cutting fashion. The present patent application embodies a group of needles which effectuate entrance into a skin surface in a puncturing fashion which is much less intrusive and much less traumatic than the imposition of a cut in the skin. Further, the device in U.S. Pat. No. 4,157,086 embodies only one cutting part, and that part can be repeatedly used. This fact means it can lead to unwanted infections when used on different subjects or at different times. The present patent application embodies a group of needles each of which can be used but once, thereby limiting the possibility of infections.

### DESCRIPTION OF THE RELATED ART

There are numerous types of devices made for performing the task the present invention performs. There are single-use devices consisting of nothing more than a needle, sometimes surrounded by a holding device. There are single use, short, pencil-like devices in which a needle is shielded until a time when the needle is released and driven by the action of a spring into a patient's skin. There are multiple-use devices made in the shape of a large-diameter pencil, and holding a few needles released one at a time from a shielded position and thrust by spring-driven action into a patient's skin. There are multiple-use devices containing a wheel which carries radially-arranged, shielded needles which can be driven into a person's skin.

The inventor of the present invention chose to make his invention one containing a wheel carrying a plurality of radially-arranged needle carriers, each of which can be driven radially by spring action to perform a skin puncturing operation.

There are at least two similarly-constituted, patented devices, but their components and operations are radically different in nature to the present invention. The only similarities between those other devices and the present invention are the ability to make skin punctures, the use of a cased wheel to carry the puncture-making components, and the use of a spring to project the puncturing components. None of those similarities are now a novelty, nor non-obvious, in the field of lancet devices. There are multitudes of patented needle-equipped lancets. There are at least two patents which use a wheel to carry radially-mounted lancet points. And many lancet patents feature the use of a cocked spring to activate a puncturing needle.

The present invention contains a number of new concepts which are new, non-obvious, and which were created solely in the mind of the present inventor, thus meeting the criteria for patenting. Those concepts will be shown on the accompanying drawings, and discussed in the text accompanying this patent application.

The present inventor lists in his utility patent application several representative patents by number and name which he believes a person might wish to investigate for comparisons to the present inventors present invention. However, the present inventor knows of no patented invention which bears a substantial resemblance to the present inventor's present invention.

### STATEMENT REGARDING FEDERALLY SPONSORED RESEARCH OR DEVELOPMENT

Not Applicable

### REFERENCE TO SEQUENCE LISTING A TABLE, OR A COMPUTER PROGRAM LISTING COMPACT DISC APPENDIX

Not Applicable

### BACKGROUND OF THE INVENTION

1. Field of the Invention

The present invention, the Renewable Rotary Skin Lancet, pertains to the class of needle-bearing devices and equipment usable by medical practitioners, persons, and patients for the purpose of puncturing a person's skin as a means of obtaining droplets of fresh blood to be used in making analytical tests. The present invention provides the equipment and a plurality of renewable puncturing needles whereby such punctures can be made safely and antiseptically a plurality of times by a broad range of users by simply cocking and firing the equipment. Then, when its supply of puncturing needles has been exhausted, a new wheel containing a plurality of new puncturing needles can be exchanged for the used wheel containing the used puncturing needles, and the Renewable Rotary Skin Lancet can continue to safely and antiseptically perform its function time after time.

2. Description of the Related Art

[a] See also, herein, the section Cross References to Related Applications, specifically the comparison there between the present invention and previously patented lancets.

[b] Many previously patented lancet devices are designed so a fresh needle-bearing part must be inserted at the time of each use. There are several problems connected with such systems. The primary problem is that during the course of preparing the skin for puncturing, readying a blood analyzer device, and loading a new needle-bearing part, it is quite easy to remember whether or not a new, antiseptic, unused, needle-bearing part has been inserted in the lancet device. The visual appearance of a used and an unused needle when seen by unassisted eyes is precisely the same. The user of such a device may simply overlook the need to insert a fresh needle-bearing part. This can be hazardous to the person whose skin is being punctured, for the needle point of a used needle can have been, during the course of making a prior puncture, or during an intervening time period, contaminated with microscopic antigens or bacteria, possibly leading to dangerous infections. That hazard could be much greater, and more significant if the lancet device being used is used not only on one person, but on a group of persons, as, for example, in a nursing home where an inordinately-high percentage of the occupants are going to be diabetics. Also, the point of an unprotected needle, during the time it resides in its lancet device between usings, may have been damaged to the extent a hook has actually been formed on the delicate, sharpened point, thereby causing an imperfect puncture, and, quite possibly, a dragging out of live tissue from the puncture site, an injurious action. Additionally, loose needle-bearing parts, as marketed by drug stores, although new and unused, may because of their boxed, unwrapped, continually exposed nature, be subject continually to the foregoing hazards. Further, such needle bearing parts subject the user and/or the assistant to the chance of an accidental puncture before, during, and after the actual intended puncture. Imagine you are the nurse administering to a diabetic hepatitis, AIDS or HIV patient. The very thought scares the present inventor, and situations such as that do occur, regularly. The known existence of such dangers as are named above prompted the present inventor to design the present invention.

The present invention is designed to eliminate the hazards described just preceding. The needle points used in the present invention are shielded from human touch and physical damage from the time they are manufactured until the time their needle wheel is disposed of, except at the moment of an intended skin puncture. It is true that contamination or blunting of the needle points can have occurred before the needle wheels are sterilely packaged for distribution, but federal authorities may have programs overseeing such manufacturing operations, and it is hoped that in no case would a manufacturer of such products allow such problems to exist in their operations. But once the needle wheel of the present invention is in the hands of a user, it would take a deliberate action to expose a needle point wherein it might be blunted, contaminated, or cause an accidental puncture.

The present inventor knows of no other lancet device having the safeguards against the preceding, named hazards as are designed into the present invention.

Even so, the present inventor, personally a diabetic who draws his blood at least four times daily, takes additional precautions against the possibility of sustaining infections at his skin-puncturing sites by disinfecting with rubbing alcohol his skin, any exposed needles, and the exposed parts of the lancet devices where they will touch his skin. Not all patients will be given or will take such precautions, so, in the opinion of the present inventor, it is quite important that the needles used for skin punctures be non-reusable, kept sterile, and not used on a plurality of patients.

[c] There are presently being marketed several lancet devices best described as throwaways. The present inventor does not know whether any, none, or all of these have received patents. They are ideal for hospitals, doctor's offices, and phlebotomist laboratories, for they can easily be kept sterile, then used once, and finally discarded. However, for a person needing a lancet device several time daily, the inconvenience of keeping a boxful of these on hand, the extra cost, and the disposal factor weigh heavily in favor of having one lancet device for each patient, provided it embodies a large supply of needles.

The lancet device for which a patent is being asked for by the present inventor will more ideally suit the needs of a person requiring the continual use of a patent device because of the convenience, sterility, and cost factors.

[d] There is presently being marketed a lancet device in which six needle-bearing devices are installed. The present inventor is unaware of there being a patent issued for said device, but, on the chance that a patent has been issued, And because it is being used by the general public, and thus it will be a competitive product for other lancet devices, it should be noted here that the present invention differs in almost every respect from said six-needle lancet device. In the opinion of the present inventor that lancet device has at least two shortcomings. First, the device holds only six needles, so, based on four punctures being performed daily, the cartridge is good for only 1½ days. Secondly, there is no fail-safe system to prevent the needles in the cartridge from being used over and over. This could lead to finger infections. It also makes it possible for the same needle to be used on more than one person. Again, the threat of diseases such as hepatitis, AIDS, and HIV infections makes such a prospect quite fearful.

The present invention can hold many needles, the number depending on the diameter of its needle wheel. The inventor estimates it will be practicable for there to be between 60 and 100 needles in the present invention's needle wheel. A new needle wheel should last 15 to 25 days when used four times daily. Additionally the present invention has a fail-safe feature, whereby no needle can be reused, even by accident.

[e] There exists a patent for a multi-needle lancet device which contains several needles, and where together the needles make a skin puncture at the same time. Using this device will likely get blood flowing, but is overly productive for normal use, and it will create several holes in the target skin. It is possible for that device to be used time after time, with the attendant possibility of infections being transmitted. Again, the hepatitis/AIDS/HIV factor must be recognized.

The present invention safeguards against the reuse of lancet needles.

[f] There exists a patent for a lancet device which includes several puncturing devices mounted on a wheel. That device uses knife-like cutters to puncture skin. Each cutter can be used time after time to draw blood, but the patient would suffer from overly extensive lacerations, and the chance of infections in the patient from repetitive uses would be objectionable.

The present invention safeguards against the hazards present in that existing patent's lancet device.

[g] There exists patents for lancet devices using a rotating knife-like cutter to puncture skin. Each cutter can be used time after time to draw blood, but the patient would suffer from overly extensive lacerations, and the chance of infections in the patient would be objectionable.

The present invention safeguards against the hazards present in that existing patent's lancet device.

[h] There exists a patent for a lancet device wherein the needle-bearing parts are mounted radially on a wheel. It appears that only six such needles can be installed on a single wheel, and it does not appear that the wheel can be replaced when the needles on it have been used. Said lancet device apparently has other shortcomings. Neither the drawings nor claims for said invention are extensive enough to be certain about what that invention included, and such details as are not shown or explained by the patent papers cannot be considered to exist. Other shortcomings are these: The puncture the device makes is within an indentation in the rim of that device, and this is not conducive to its use on flat skin areas such as are preferred by some patients needing the use of a lancet device; There is no means of adjusting the depth its needles penetrate, and such adjustments are needed for different types of skin; The patent suggests that the needle-carrying parts are to be inserted in the wheel by hand, and this presents the possibility of finger sticks and contamination of the needles; The patent suggests that a used needle can remain with its point hazardously exposed after the lancet has been used, and this can be dangerous; The needles in said device must employ an entirely separate device to pull off protective covers on the tips of the needles in the lancet device, and this can lead to problems such as a lost pulling device, blunting of the fragile needle point during the process, and infection of the needle through the presence of contaminated material in the separate, pulling device; The patented device has protruding parts making it unwieldy in both the hand and in storage; The patented device will not delineate the puncture point when it is used, so the exact point of puncture, if it needs to be squeezed to start the blood flowing, cannot be easily determined, and this may mean additional punctures must be made; The patented device is overly complicated for use by patients having borderline mental or physical abilities, and is thereby not conducive to being used by a broad array of people; The needle-bearing parts of the patented device are not made so that, once used, they cannot be reused; and The patented device does not make use of a secondary needle-centering device, so its needle's precise location may be questionable and erratic.

In comparison to the shortcomings of the patented device, the present invention has these attributes and differences: A replaceable needle wheel; A detent system for positively centering its needles at the needle aperture; Needles which are shielded from human touch except when a puncture is intended to occur; Can be used on flat or curved areas of skin; Contains a mechanism for closely adjusting the penetration depth of the needle points; Makes use of factory loaded needle wheels; Does not require the use of a separate device for pulling off protective covers on the needles; Will indent the skin visibly in order to see where a puncture has occurred; Cannot reuse the needles; Some of its needle carriers are made wherein a secondary backup to non-reuse of its needles is built in; Requires fewer steps for its use; Can be successfully used by a greater proportion of the population.

Additionally, the needle-propulsion system, the wheel rotation system, and the firing mechanism on the patented lancet device all differ greatly from those on the present invention being submitted for patenting.

### BRIEF SUMMARY OF THE INVENTION

The Renewable Rotary Skin Lancet is a device designed to allow the puncture of the skin of medical patients for the purpose of safely and antiseptically obtaining small amounts of their blood, usually as a preliminary to using said blood for testing purposes, and to do so with the least practicable amount of danger, discomfort, cost, outside assistance, and effort.

The Renewable Rotary Skin Lancet is comprised of a mechanism contained in a case which may be easily opened by the user. The mechanism is comprised of a circular wheel containing needle carriers, each of which contains a pointed needle. A mechanism is provided whereby the user may propel the needles on the needle carriers, one by one, into the patient's skin. A cocking procedure is required after each needle carrier is used, and this cocks the propulsion mechanism while rotating the needle-carrying wheel so that a new, unused, needle is moved into a position where it may be propelled next. After all the new, unused, needles on the wheel have been used one time the needle-carrying wheel can no longer be made to rotate. Then, so the device can continue to be used, the used wheel can be removed, and a new needle-bearing rotary wheel can be inserted into the device, making the device again ready for use.

The present invention is intended for each of the devices to be used on one person. Even though the chance of cross infection would be small if it were used on more than one person, that possibility should be avoided. The device is designed with the intention that the user is also the person whose blood is being tested, but, of course, an assistant can also make the puncture if that need exists.

It is the primary objective of the present invention, the Renewable Rotary Skin Lancet, to provide a safe, convenient, and easily used lancet device for obtaining small amounts of blood from a broad array of persons.

Largely due to the growing prevalence of diabetes across all ages, races, genders and sizes of people, there is a growing need for a lancet device which can be used with safety by a diverse group of persons. People who are young, disabled, incapacitated, or ill may need, irrespective of their limitations, a lancet device which they can personally use without the possibility of accidental blood contamination from unwanted needle punctures, dirty needles. The present invention has needle points which are, with normal use, unable to be touched except at the time of each intended puncture, and, because of this, it stands almost alone in its safe shielding of its plurality of needle points up to and after the time a purposeful skin puncture is made. The present invention promotes the safe use of lancet devices.

Whereas, after each use of a single-use lancet device, such a device should be disposed of in a sharps container, this is not necessary with the present invention, for, even after a renewable needle wheel has be completely used, the needle points of the throwaway wheel are not exposed to being touched.

In the event the present invention encounters some possibly-infectious environment, it ordinarily may be sterilized by immersion in an antiseptic mix of alcohol. As an additional precaution, the present inventor recommends using an alcohol pad to wipe and sterilize the needle aperture area of this or any lancet device prior to each use. During normal blood testing there will be an alcohol-soaked pad used in other parts of the testing, and this will be available for fulfilling this extra safety step.

When the present invention is used in group facilities such as nursing homes or hospitals, it is important that the name of the person to whom the use of the device is consigned be written with an indelible marker directly on the device as a means of segregating the device to the use of only one individual. There is space provided on both sides of the present invention for this to be done.

The size of the present invention is larger than some other lancets, but it is small enough to fit in the palm of one hand, yet large enough to keep it from being easily misplaced.

Because there is a plurality of numbered needle carriers in the present invention, its supply of needles will last much longer than other types of lancets, and, at any time, the user can see what quantity of needles remains available for use. Once the present invention is equipped with a new needle carrier wheel, all that remains for putting it to use is pushing the triggering button to fire the device, then cocking the device before the next use. With the exception of some juveniles, the present invention should be able to be used without assistance by all persons having near-normal physical and mental capacities.

Some other lancet devices have some of the advanced features of the present invention. The present inventor knows of no other lancet device which has all of the advanced features of the present invention.

Following is a list of some of the problems present in at least one or more other lancet devices. These problems have been solved in the design of the Renewable Rotary Skin Lancet.

1. The needles in the other device(s) are able to be reused, possibly leading to infections or damaged needle points.

2. The other device(s) are too cumbersome or their use too involved to be used personally by some persons having reduced capabilities.

3. The other device(s) incorporate the use of either only one or only a few needles, thus requiring extra time and effort to make the device(s) ready for use.

4. The other device(s) cut the skin instead of puncturing it, thereby causing an unnecessary, possibly harmful, laceration when obtaining blood samples.

5. The other device(s) have their needle points exposed for a period of time, other than at the moment of puncture, during the use of the device(s), possibly leading to needle contamination or needle damage.

6. The other device(s) have their needle points exposed for a period of time during the readying or use of the device(s), possibly resulting in accidental needle punctures and a resulting disease.

7. The other device(s) incorporate no provision for adjusting the depth of penetration of their needles during a puncturing procedure.

8. The other device(s) leave a needle point exposed after their use, necessitating, if good sanitation is practiced, the use of a sharps container.

9. The other device(s) would be subject to damage if they were immersed in rubbing alcohol, making it necessary to throw them away if contamination is suspected.

10. The other device(s) cannot be marked with the name of the person to whom they are consigned.

11. The other device(s) require the removal of a protective cover from their needles before they can be used, thereby increasing the time and effort for their use and subjecting the user to a possible unwanted skin puncture while removing the cover.

12. The other device(s) require the use of a separate, preparatory device in order for the lancet device to be used.

13. The other device(s) are an unwieldy shape or size, making them awkward to handle and carry, difficult to store, confusing to use, or so small as to be easily misplaced.

14. The other device(s) do not delineate the point of puncture with a short-lived impression in the skin when a puncture is made, possibly necessitating follow-up punctures if the puncture site should be squeezed but cannot be found.

15. The other device(s) do not have needle carriers which, once used, will refuse to be propelled for another use, thereby denying the user a backup to any first line of reuse rejection the device may have.

16. The other device(s) can only be used on a tightly curved area of skin, such as a finger or toe, and this excludes their use by persons seeking to puncture a flatter area of skin, such as an arm or a thigh.

17. The other device(s), where a plurality of needles is stored in the device, lack a means of quickly determining what quantity of unused needles remain in the device.

### IDENTIFICATION AND DESCRIPTION OF THE FIGURES ON THE DRAWINGS FOR THE RENEWABLE ROTARY SKIN LANCET

FIG. 1 Oblique view of the lid side of the Case for Alternate Device No. 1. The Case is used to contain the entire working mechanism of the device. The Case serves to minimize the intrusion of unwanted matter in the working mechanism, which must be kept as sterile as possible. There will be three unsealed openings in the case. The Aperture 1D for the Needle projection. The Opening 1A for the triggering Pushbutton 5. And the Opening 1B for the Cocking Lever 4G. There will be some projecting parts molded to the inner surface of the Case Lid 2 and the Case Bottom 1. The Case Lid and the Case Bottom may be made of either some sort of plastic or some sort of metal.

FIG. 2 Oblique view of the bottom side of the Case for Alternate device No. 1.

FIG. 3 Plan view of the lid exterior of the Case for Alternate Device No. 1.

FIG. 4 Plan view of the lid interior of the Case for Alternate Device No. 1.

FIG. 5 Plan view of the Lid attachment screws 2B. These serve to hold the Case Lid on the Case Bottom. It is highly likely that these screws will be replaced in the actual manufacturing process by some sort of fastener which can be operated by the user's fingers, and there may be a hinge of some sort to keep the Case Lid and Case Bottom attached. The capability of the manufacturer will dictate the fastening used. The screws, if such are used, may be made of some sort of plastic or some sort of metal.

FIG. 6 Side view of the Lid attachment screws.

FIG. 7 Section through the perimeter of the lid for the Case of Alternate Device No. 1. The Offset 2A in the meeting-edges of both the Case Lid and the Case Bottom merely serve to help keep those two parts firmly attached together in the proper relationship to each other.

FIG. 8 Plan view of the exterior of the bottom side of the Case for Alternate device No. 1.

FIG. 9 Plan view of the interior of the Case Bottom for Alternate Device No. 1. The Centering Notch 1E is used to mark the location of Needle-Carrier number 1 when a new Needle-Carrier Wheel is inserted. The Lid-Attachment Screw Posts 1H are shown, and these serve also as spacers to keep the Case Lid the proper distance from the Case Bottom so the Case Lid does not keep the Needle-Carrier Wheel from rotating freely. The Carrier Alignment Posts 1G are shown. These fit snugly, but freely, in Alignment-Post Openings 3S located in the Wheel Carrier 3, and serve to keep the Wheel Carrier laterally stable while still permitting it to slide towards and away from the Needle Aperture 1D when the Needle's penetration depth is being adjusted.

FIG. 10 A view of the Case Bottom for Alternate Device No. 1 while looking at its needle aperture end.

FIG. 11 Longitudinal section through the Case Bottom for Alternate Device No. 1.

FIG. 12 A view of the Case Bottom for Alternate Device No. 1 while looking at its adjusting knob 3D end.

FIG. 13 Plan view of the inside of the Wheel Carrier 3A for Alternate Device No. 1. The Wheel Carrier 3 serves as a platform for the working mechanisms of the device. Use of the Wheel Carrier allows the working mechanism to be moved towards or from the Needle Aperture 1D of the Case, and this makes possible the adjustment of the depth of penetration of the Needle 7A when it is used for puncturing skin. The Wheel Carrier may be made of some sort of plastic or some sort of metal.

FIG. 14 Longitudinal section of the Wheel Carrier 3A for Alternate Device No. 1.

FIG. 15 View of the outer end of the Adjusting Arm 3P on the Wheel Carrier 3. The Adjusting Arm will be an integral part of the Wheel Carrier, made of the same material, and it serves as a part into which the Adjusting Stud 3A will be threaded.

FIG. 16 Face view of the Upper Hammer Guide 3H showing the Hammer opening 3L and the Push Button Spring opening 3J. Both Hammer Guides are essentially the same, but the Upper Hammer Guide contains an opening through which the Pushbutton Spring will pass and be flexed. The Hammer Guides will prevent lateral movement of the Hammer as it is operated. The Hammer Guides will serve as retainers for the cylindrical compression springs 4A and 4B surrounding the Hammer. The Hammer Guides will be an integral part of the Wheel Carrier 3H, and made of the same material

FIG. 17 Plan view of the Upper Hammer Guide 3H.

FIG. 18 Side view of the Upper Hammer Guide.

FIG. 19 Face view of the Lower Hammer Guide 3K.

FIG. 20 Plan view of the Lower Hammer Guide.

FIG. 21 Side view of the Lower Hammer Guide.

FIG. 22 Face view of the Cocking Lever Stop Arm for Alternate Device No. 1. This part is an integral part of the Wheel Carrier. It serves to limit the axial movement of the Hammer when the Hammer is cocked.

FIG. 23 Plan view of the Cocking Lever Stop Arm 3M for Alternate Device No. 1.

FIG. 24 Side view of the Cocking Lever Stop Arm for Alternate Device No. 1.

FIG. 25 Plan view of the bottom of the Wheel Carrier 3 for Alternate Device No. 1.

FIG. 26 Longitudinal section of the Renewable Rotary Skin Lancet, Alternate Device No. 1 including applicable parts numbers. This version shows the Hammer 4, cocked and ready to fire.

FIG. 27 Longitudinal section of the Renewable Rotary Skin Lancet, Alternate Device No. 1 with parts numbers removed for clarity of drawing details. This version shows the Hammer fired, and the Needle in its puncturing position.

FIG. 28 Longitudinal section of the Renewable Rotary Skin Lancet, Alternate Device No. 1 with parts numbers removed for clarity of drawing details. This version shows the Hammer as it is being cocked, and with the Needle partly retracted after being used to make a skin puncture.

FIG. 29 This shows a section through the Wheel 6 of Alternate Device No. 1 with a Needle Carrier 7 in its pre-fired or retracted position. The Wheel contains the Needle Carriers in the Needle Slots 6B. The Wheel 6 and the Needle Carriers 7 may be made of plastic or metal, and there must be little friction between the Wheel and the Needle Carriers so the Needle Carriers can easily be impelled. The Wheel has a Rubbing Rail 6A which is perpendicular to the main plane of the Wheel, and this seats over a counterpart 3R Rubbing Rail on the Wheel Carrier 3 to keep the Wheel 6 properly aligned and smoothly rotational.

FIG. 30 This shows a section through the Wheel 6 of Alternate Device No. 1 at an Advancer Pit 6G. The Advancer Pit is a recess into which the Advancer 4K will be thrust when the Hammer 4 is cocked. The Advancer Pit serves as a sort of cog, and is used to move the Wheel 6 along its rotational path.

FIG. 31 This shows a section through the Wheel 6 of Alternate Device No. 1 at a Detent Notch 6E. The Detent Notch is used as a seat for the Detent Protrusion 3Y. Together, the Detent Notch and Detent Protrusion serve to keep the Needle Carriers centered on the Needle Aperture 1D.

FIG. 32 A plan view of the Wheel of Alternate Device No. 1 showing the Stop Post 6D and the direction of rotation. The Stop Post is an integral projection on the Needle Carrier Wheel 6. Its counterpart is the Wheel-Stop Tab 3W which is an integral part of the Wheel Carrier. When a new Wheel 6 is inserted in the device the Stop Post will be on the leading side of the Wheel-Stop Tab. When all the Needle Carriers in the Wheel have be used once, the Stop Post will meet the Wheel Stop Tab, and will then disallow any further rotation of the Wheel, and, thereby, any further use of the now-used Needles.

FIG. 33 A plan view of internal sections of the Wheel of Alternate Device No. 1 showing the Needle-Carrier Slots 6B, Detent Notches 6E, and entry-exit spaces 6G and 6H for the Probe when it is in a fired state. The Wheel contains Needle-Carrier Slots 6B within which the Needle Carriers 7 are located. The Needle Carriers contain the Needles 7A used for making skin punctures. The Probe Slot for Wheel Insertion 6G is an entry space in the Wheel used to receive the Probe when it is in a fired state, as it must be so the advancer 4K will be clear of the Advancer Pits 6C at the opposite end of the Wheel when a new Wheel is inserted. The Probe Slot for Wheel Removal serves a similar purpose when the used Wheel is removed.

FIG. 34 Another plan view of internal sections of the Wheel of Alternate Device No. 1 showing the Needle Carrier Slots, Detent Notches, and entry-exit spaces for the Probe when it is in a fired state.

FIG. 35 A section through one edge of the Alternate Device No. 1 showing the Stop Post, Detent Ramp, and Detent Leaf. The detent Ramp is an integral part of the Wheel Carrier. It serves to elevate the Detent Leaf to a usable height, The Detent Leaf and its Detent Protrusion are one part. The Detent Leaf acts as a spring to keep its Protrusion tightly against the bottom side of the Wheel. The Detent Leaf and its Protrusion 3Y may be made of either plastic or of metal.

FIG. 36 A view from its anchoring end of the Detent Leaf and its Protrusion 3Y of the Alternate Device No. 1.

FIG. 37 A view from its anchoring end of the Detent Leaf 3Y and Detent Ramp 3X showing a part of the Wheel 6 of the Alternate Device No. 1 in the background.

FIG. 38 A section through one edge of the Alternate Device No. 1 showing the Stop Post 6D and Wheel Stop Tab 3W.

FIG. 39 A detail at one edge of the Alternate Device No. 1 showing the Stop Post and Wheel Stop Tab after a new Wheel has been inserted, with a part of the Wheel in the background. The wheel is now ready to be used from its first Needle.

FIG. 40 A plan view of the Detent Leaf and Detent Ramp of the Alternate Device No. 1.

FIG. 41A plan view showing the Stop Post and Wheel Stop Tab of the Alternate Device No. 1 after a new Wheel has been inserted.

FIG. 42 A plan view of the Wheel of the Alternate Device No. 1 showing the entry and exit spaces in the bottom side of the Wheel to allow insertion and removal of the Wheel at the beginning and end of its use when the Probe and Hammer are in their fired position. Also shown are the numbers 8 on the Wheel indicating the number of the Needle Carrier 7 positioned beneath the number. The numbers, and any other markings, arrows, or instructions are to be of lasting quality, not destructible by any alcohol used for sterilization purposes, and clearly visible on whichever surface they are installed.

FIG. 43 A section through one edge of the Wheel of Alternate Device No. 1 without any descriptive lettering, shown for clarity of design.

FIG. 44 A view of the inner perimeter of the Wheel of the Alternate Device No. 1 showing the entry and exit slots for the Probe when the Wheel is being inserted or removed.

FIG. 45 A section through one edge of Alternate Device No. 1 without any descriptive lettering, shown for clarity of design.

FIG. 46 A detail at one edge of the Alternate Device No. 1 showing the Stop Post and Wheel Stop Tab after a Wheel has been completely exhausted of new Needles, with a part of the Wheel in the background. The Wheel is ready for replacement.

FIG. 47 A plan section of the inner perimeter of the Wheel of the Alternate Device No. 1 showing the entry and exit slots for the Probe for use when the Wheel is being inserted or removed. The Hammer must be in a fired position for the Wheel to be successfully inserted or removed. This creates the need for these slots. If the Hammer is not fired, the Advancer would be inside one of its pits, and would restrict insertion or removal of the wheel. If the Hammer is fired, the Probe/Retractor would be inside a Needle Carrier Slot in the Wheel except when the Probe/Retractor is at the designated insertion and removal slots 6 G and 6H in the Wheel.

FIG. 48 A plan view at one edge of the Alternate Device No. 1 showing the Stop Post and Wheel Stop Tab after a Wheel has been completely exhausted of new Needles. The Wheel is ready for replacement.

FIG. 49 The plan view of the Hammer 4 of the Alternate Device No. 1. The hammer may be made of plastic or metal. Its purpose is to act as a weighted mass which will properly carry the probe/retractor toward a Needle Carrier, strike the Needle Carrier, and impel the Needle Carrier towards the skin of the target person. It must fit snugly but not bound within its Guides, and be nearly without friction so it can travel easily and without lateral deviation along its longitudinal axis. The Hammer may need weight added or subtracted so that it will have the proper balance of momentum and inertia, and coordinated with the springs surrounding it, it will capably perform its intended functions.

FIG. 50 The left side view of the Hammer of the Alternate Device No. 1.

FIG. 51 The face view of the Hammer of the Alternate Device No. 1.

FIG. 52 The right side view of the Hammer of the Alternate Device No. 1.

FIG. 53 The bottom view of the Hammer of the Alternate Device No. 1.

FIG. 54 A side view of the Advancer 4K of the Alternate Device No. 1 after it has been moved by the Cocking Lever 4G to advance the Wheel 6 forward one Needle Carrier Space. The Advancer is used to rotate the Wheel. It may be made of either plastic or metal. It is fastened to the Hammer in a way so it can be removed for repair or replacement. The extent of its action is governed by adjustment screw 30 (three-Oh). If there is some malfunction of the operating mechanisms during their use, it may be necessary to remove the Advancer so the Wheel can be removed, for the Probe/Retractor may not be in its Slot for Wheel Removal 6H at the time of malfunction. The leaf part of the advancer must be flexible, but the degree of flexibility must be finely governed to be sure the Advancer performs properly. There is an angled hump on the Advancer, and the purpose of this is to cant the loose end of the Advancer in the direction of rotation of the Wheel 6, thereby, with its push on the side wall of an Advancer Pit 6C, moving the Wheel 6 along its intended path of rotation by the proper distance for bringing a new Needle Carrier into the position in which it can be impelled towards the target's skin. There will be a tendency for the loose end of the Advancer to drag the Wheel 6 backwards, opposite its intended rotational path, as the Advancer returns to its static position, and the Detent with Protrusion 3Y is intended to firmly anchor the Wheel once the Wheel has been advanced to place a new Needle Carrier in a usable position. The Protrusion of 3Y may need to be made with a slope on the following face and a vertical face on the leading face, with a complimentary shape used on the Detent Notches 6E, to firmly anchor the Wheel 6 when it has reached its proper advancement. If the Detent Leaf 3Y needs to be more effective, its orientation may need to be revised so any tendency of the leaf part of Detent Leaf 3Y to twist or crumple will be forestalled.

FIG. 55 A side view of the Advancer 4K of the Alternate Device No. 1 either before or after it has actuated the Wheel 6.

FIG. 56 A face view of the Advancer of the Alternate Device No. 1 either before or after it has actuated the Wheel.

FIG. 57 A side view of the Advancer of the Alternate Device No. 1 by itself.

FIG. 58 A face view of the Advancer of the Alternate Device No. 1 by itself.

FIG. 59 A plan view of the Advancer of the Alternate Device No. 1 by itself.

FIG. 60 A knob end view of the Adjusting Knob 3D when assembled. This knob is attached to the Wheel Carrier 6 via a Threaded Stud 3A. Turning the Adjusting Knob moves the Wheel Carrier 3 towards or away from the Needle Aperture, and, thereby, adjusts the depth of the Needle's skin penetration. The Knob and its associated parts may be made of either plastic or metal. A Washer 3B will be used to reduce friction. A partially-protruding Pin 3C may be needed to retain the Washer. Although not shown, a second Washer 3B may be needed between the Knob 3D and the case containing the mechanisms.

FIG. 61A plan view of the Adjusting Knob parts when assembled.

FIG. 62 An Adjusting Stud end view of the Adjusting Knob parts when assembled.

FIG. 63 A section through one edge of the Alternate Device No. 1 showing the use of Alternate Needle Carrier 7 and Alternate Probe 4E. The Needle is in its puncturing position. Several alternate Needle Carriers and Probe/Retractors designed to function with their companion Needle are shown on the drawings accompanying the application for patenting the present device. The type chosen for actual use will be made when the capabilities of the selected manufacturer is known and the best embodiment of the Renewable Rotary Skin Lancet has been chosen. Although the various Needle Carrier types are shown with cylindrical or oval-sectioned bodies, most of them may be made also of rectangular-sectioned bodies. The present inventor is applying for a patent which includes all of the Needle Carrier types. Needle Carrier 7 includes two flexible Wings 7B which have at their ends Stop Protrusions 7C, the purpose of which is to limit the distance the Needle Carrier can travel in its Needle-Carrier Slots 6B. There are Retraction Protrusions 7C on the inner surface of the Wings 7B, the purpose of which is to fit into the Retractor Slot 4F on Probe/Retractor 4E, the companion Probe/Retractor for Needle Carrier 7, when the Probe/Retractor 4E is thrust into the Needle Carrier, and the Needle Carrier is impelled, and to provide an anchor so the Needle Carrier can be retracted. Once the Probe/Retractor, and connected Needle Carrier have been retracted a certain amount, the Retraction Protrusions 7C will let the Probe/Retractor continue to withdraw by itself and without the Needle Carrier. The Wings 7B on the Needle Carrier 7 will be formed in a spread shape, the shape it needs to assume upon retraction, so they will spread out when not bound inside the Needle-Carrier Slot 6B.

FIG. 64 A face view of Alternate Needle Carrier Type 7 of the Alternate Device No. 1. This Needle Carrier, its companion Probe/Retractor 4E, and all other Needles and their companion Probe Retractors shown in the present patent application may be made of either plastic or metal. The Needles in all those Needle Carriers will be cylindrical rods made of metal, sharply pointed at their protruding end, anchored firmly in the Needle Carrier, and of the smallest diameter consistent with minimal skin damage, but a large enough diameter to withstand bending forces which may normally be present while being used to puncture the target's skin. Each of the combination of companion Needle Carriers and Probe/Retractors is designed to function together performing their functions of impelling the Needle Carrier to a location where a target's skin can be punctured, then partially retracting the Needle Carrier while leaving the Needle Carrier in the Wheel within which it resides. Although several of the Needle Carriers are designed so that once they are used a blocking mechanism prevents their reuse, Needle 7 features no such blocking mechanism. However, any reuse would entail a malfunction of some sort, and reuse will, absent such a malfunction, be prevented by the introduction of a new, unused Needle Carrier each time the device's Hammer is cocked. This should provide the safety factor required, since a malfunction will probably result in the Hammer being unable to be cocked, or no new Needle Carrier being advanced in rotation of the Wheel. This Needle Carrier has no blocking mechanism to provide a second line of defense against its accidental reuse. Nevertheless, its use may be warranted because of its simplicity.

FIG. 65 A side view of Alternate Needle Carrier 7 of the Alternate Device No. 1.

FIG. 66 A section through one edge of the Alternate Device No. 1 showing the use of Alternate Needle Carrier 7 and Alternate Probe Type 4E. The Needle is shown after its retraction. It would look the same when new and ready to be activated.

FIG. 67 A face view of Alternate Probe/Retractor 4E of the Alternate Device No. 1. The application being made by the present inventor includes several types of Probe/Retractors. This is due to the fact that different needle types require different Probe/Retractors. The purpose of the Probe/Retractors is to strike the Needle Carriers when they are in their proper position to have their Needles impelled into the target's skin. The Probe/Retractors may be made of plastic or metal.

FIG. 68 A plan view of Alternate Probe 4E of the Alternate Device No. 1.

FIG. 69 A side view of Alternate Probe 4E of the Alternate Device No. 1.

FIG. 70 A section through one edge of the Alternate Device No. 1 showing the use of Alternate Needle Carrier 9 and Alternate Probe Type 10. The Needle is in its puncturing position. This Needle Carrier has no blocking mechanism to provide a second line of defense against its accidental reuse. In this version of Needle Carrier/Probe/Retractor, the Needle Carrier is equipped with Jaws 9C, and the Probe/Retractor 10 includes a cap onto which the Jaws of the Needle Carrier will be clamped in the process of being used. Stop Shoulders 9B on the Needle Carrier will serve to limit its travel towards the target. The Wings 9A will be flexible, and will be spread when not bound. Much of what has been said about Needle Carrier 7 and Probe/Retractor 4E applies also to Needle Carrier 9 and Probe/Retractor 10.

FIG. 71A face view of Alternate Needle Carrier 9 of the Alternate Device No. 1.

FIG. 72 A side view of Alternate Needle Carrier 9 of the Alternate Device No. 1.

FIG. 73 A section through one edge of the Alternate Device No. 1 showing the use of Alternate Needle Carrier 9 and Alternate Probe 10. The Needle is shown after its retraction. It would look the same when new and ready to be activated.

FIG. 74 A face view of Alternate Probe 10 of the Alternate Device No. 1.

FIG. 75 A plan view of Alternate Probe 10 of the Alternate Device No. 1.

FIG. 76 A side view of Alternate Probe 10 of the Alternate Device No. 1.

FIG. 77 A section through one edge of the Alternate Device No. 1 showing the use of Alternate Needle Carrier A1 and Alternate Probe A2. The Needle is shown when new and ready to be activated.

FIG. 78 A section through one edge of the Alternate Device No. 1 showing the use of Alternate Needle Carrier A1 and Alternate Probe A2. The Needle is shown at the moment it is making a puncture. Alternate Needle Carrier A1 has been designed with a Shoulder A6 which is intended to prevent any reuse of this Needle Carrier. When Needle Carrier A1 is partially retracted after being used, the Shoulder A6 will be moved outwardly as the Flexible Shoulder Arm A5 flexes outwardly to its normal shape when it is unrestricted, and Shoulder A6 will catch on the inner edge of the Wheel if any attempt is made to reuse the Needle Carrier. As with several other Needle Carrier and Probe/Retractor designs, Needle Carrier A1 and Probe/Retractor A6 will have deformations on their Caps and Jaws which will make their attachment more secure when they are functioning.

FIG. 79 A section through one edge of the Alternate Device No. 1 showing the use of Alternate Needle Carrier A1 and Alternate Probe A2. The Needle Carrier and Needle is shown after it has been used for a puncture, after it is released from the Probe/Retractor, before the Probe/Retractor has been completely retracted, and the wings of the Needle Carrier have spread apart, with the wing's shoulders preventing any reuse of the used Needle Carrier and its Needle.

FIG. 80 A face view of Alternate Needle Carrier A1 of the Alternate Device No. 1.

This version of the Needle Carrier features two sets of shoulders. The shoulders at the Jaw stops the Needle Carrier from excess travel when those shoulders hit the Wheel after the Hammer has been fired. The shoulders on the Needle Carrier Wing are inside the Needle Carrier Slot before being fired, but are exposed after the Needle Carrier's retraction, and are then in a position to disallow reuse of the used Needle Carrier and its Needle.

FIG. 81A side view of Alternate Needle Carrier A1 of the Alternate Device No. 1.

FIG. 82 A side view of the Cap on Alternate Probe A2 used with Alternate Needle Carrier A1 on Alternate Device No. 1. The Cap surfaces are deformed to better grasp complementary deformations in the Needle Carriers. Such deformations are used on several of the Needle Caps, Needle Jaws, Probe/Retractor Caps, and Probe/Retractor Jaws shown in the present patent application. The deformations are matching grooves and ridges intended to help the caps center on and hold the caps without slipping laterally.

FIG. 83 A face view of the Cap on Alternate Probe A2 used with Alternate Needle Carrier A1 on Alternate Device No. 1.

FIG. 84 A view of the beveled end of the Clamping Jaw on Alternate Needle Carrier A1 used with Alternate Probe/Retractr A2 0n Alternate Device No. 1. The Beveled Ends are intended to make it easier for the Needle Caps or Jaws and the Needle caps or Jaws to meet and slip over each other when their Needle Carriers are being impelled toward the target's skin.

FIG. 85 A face view of the beveled end of the Clamping Jaw on Alternate Needle Carrier A1 used with Alternate Probe/Retractor A2 0n Alternate Device No. 1.

FIG. 86 A face view of Alternate Probe/Retractor A2 for use on Alternate Device No. 1.

FIG. 87 A side view of Alternate Probe/Retractor A2 for use on Alternate Device No. 1.

FIG. 88 A section through one edge of the Alternate Device No. 1 showing the use of Alternate Needle Carrier A11 and Alternate Probe/Retractor A2. The Needle Carrier is shown when new and ready to be activated.

FIG. 89 A section through one edge of the Alternate Device No. 1 showing the use of Alternate Needle Carrier A11 and Alternate Probe/Retractor A2. The Needle Carrier is shown at the moment of making a puncture.

FIG. 90 A section through one edge of the Alternate Device No. 1 showing the use of Alternate Needle Carrier A11 and Alternate Probe/Retractor A2. The Needle Carrier is shown after having been retracted. The Shoulders A13 on the Needle Carrier Arms A12 have dropped into the Shoulder Pockets A18 formed inside the Needle Carrier Slots within the Wheel, and the Shoulders A13 then will disallow this Needle Carrier and Needle from reuse.

FIG. 91A face view of Alternate Needle Carrier A11 of the Alternate Device No. 1.

FIG. 92 A side view of Alternate Needle Carrier A11 of the Alternate Device No. 1. The Flexible Shoulder Arm A12 must flex, but must be stiff enough to force Shoulder A13 firmly into Shoulder Pocket A18

FIG. 93 A side view of the beveled end of the Clamping Jaw on Alternate Needle Carrier A11 used with Alternate Probe A2 0n Alternate Device No. 1. The Jaw surfaces are deformed to better grasp complementary deformations in the Probes.

FIG. 94 An end view of the beveled end of the Clamping Jaw on Alternate Needle Carrier A11 used with Alternate Probe A2 0n Alternate Device No. 1.

FIG. 95 A plan view of the Push Button 5 used to release and restrain the Hammers of the Renewable Rotary Skin Lancet. The Pushbutton comprises a rectangular button 5 on which an integral square ring 5C is formed. The inner dimensions of this ring are somewhat larger than the diameter of the Hammers, for the Hammers must pass through the ring when the Pushbutton is pressed. When the Hammer is cocked the inner edge of the ring will hold the Hammer in its cocked position. The Pushbutton Spring 5A is a leaf type spring which presses the Pushbutton outwardly from the device Case. When the Hammer is cocked the Pushbutton Spring returns the Pushbutton to its initial position, and the square ring again retains the Hammer. The Pushbutton and Pushbutton Spring are fastened together, and the Pushbutton Spring is fastened to the floor of the Wheel Carrier. The Pushbutton and Pushbutton Spring may be made of Plastic or Metal. This Pushbutton 5 is used whichever of the Alternate Devices is used.

FIG. 96 An side view of the Push Button used to release and restrain the Hammers of the Renewable Rotary Skin Lancet.

FIG. 97 A face view of the Push Button used to release and restrain the Hammers of the Renewable Rotary Skin Lancet.

FIG. 98 Oblique view of the lid side of the Case for Alternate Device number 2. Alternate Device Number 2 is essentially the same as Alternate Device Number 1, but Alternate Device Number 2 uses a Rocker Arm C10 and Cog Wheel B6 method for advancing the Needle-Carrier Wheel B8. In this system the Cog Wheel and Needle-Carrier Wheel are two separate wheels bound together only by three Positioning Pins C11D. The Positioning Pins are eccentric in location so the Needle Carrier Wheel B8 can fit only in its correct location on the Cog Wheel B6, and, additionally, the Needle Carrier Wheel cannot be installed upside down. The Lancet Number Viewing Window B3 is located immediately beneath the Needle Aperture 1D.

FIG. 99 Oblique view of the bottom side of the Case for Alternate device No. 2.

FIG. 100 Plan view of the lid exterior of the Case for Alternate Device No. 2.

FIG. 101 Plan view of the lid interior of the Case for Alternate Device No. 2.

FIG. 102 Plan view of the Lid attachment screws. These are the same as those shown for Alternate Device Number 1. They too may be replaced by another fastening method.

FIG. 103 Side view of the Lid attachment screws.

FIG. 104 Section through the perimeter of the lid for the Case of Alternate Device No. 2. This is the same as is shown for Alternate Device Number 1.

FIG. 105 Plan view of the exterior of the bottom side of the Case for Alternate device No. 2. The bottom part of the Case contains the Lancet Number Viewing Opening for Alternate Device Number 2, whereas the Case Lid included that opening.

FIG. 106 View of the Case Bottom for Alternate Device No. 2 while looking at its adjusting knob end.

FIG. 107 Plan view of the interior of the Case Bottom for Alternate Device No. 2.

FIG. 108 View of the Case Bottom for Alternate Device No. 2 while looking at its needle aperture end.

FIG. 109 Longitudinal section through the Case Bottom for Alternate Device No. 2.

FIG. 110 Plan view of the inside of the Wheel Carrier for Alternate Device No. 2.

FIG. 111 View of the outer end of the Adjusting Arm 3P on the Wheel Carrier B3.

FIG. 112 Face view of the Upper Hammer Guide 3H showing the Hammer opening 3L and the Push Button Spring opening 3J.

FIG. 113 Side view of the Upper Hammer Guide.

FIG. 114 Plan view of the Upper Hammer Guide.

FIG. 115 Face view of the Lower Hammer Guide 3K.

FIG. 116 Side view of the Lower Hammer Guide.

FIG. 117 Plan view of the Lower Hammer Guide.

FIG. 118 Face view of the combination Hammer Stop and Retractor Spreader B3A for Alternate Device Number 2.

FIG. 119 Side view of the combination Hammer Stop and Retractor Spreader for Alternate Device Number 2.

FIG. 120 Plan view of the combination Hammer Stop and Retractor Spreader for Alternate Device Number 2.

FIG. 121 Plan view of the Rocker Arm Pivot Pedestal C10E for Alternate Device Number 2.

FIG. 122 Face view of the Rocker Arm Pivot Pedestal for Alternate Device Number 2.

FIG. 123 Side view of the Rocker Arm Pivot Pedestal for Alternate Device Number 2.

FIG. 124 Plan view of Pedestal C11C for Detent Dog Body C11 for Alternate Device Number 2.

FIG. 125 Face view of Pedestal for Detent Dog Body for Alternate Device Number 2.

FIG. 126 Side view of Pedestal for Detent Dog Body for Alternate Device Number 2.

FIG. 127 Longitudinal section of the Wheel Carrier 3R for Alternate Device No. 2.

FIG. 128 Plan view of the bottom of the Wheel Carrier for Alternate Device No. 2.

FIG. 129 Longitudinal section of the Renewable Rotary Skin Lancet, Alternate Device No. 2, including applicable parts numbers. This version shows the Hammer B4 cocked and ready to fire.

FIG. 130 Longitudinal section of the Renewable Rotary Skin Lancet, Alternate Device No. 2 without showing parts numbers. This version also shows the Hammer B4 cocked and ready to fire.

FIG. 131 Longitudinal section of the Renewable Rotary Skin Lancet, Alternate Device No. 2 without showing parts numbers. This version also shows the Hammer cocked and ready to fire. This drawing is the same as FIG. 130, and is shown again to provide correlation to FIG. 132 located on the same drawing page.

FIG. 132 Plan view of interior of Alternate Device No. 2 with the Case Lid removed. In this drawing the function of the Rocker Arm C10 and Detent C11 can be seen. The Rocker Arm is activated by the cocking of the Hammer B4. But cocking the Hammer does not immediately move the Rocker Arm. The cocking action must first let the Probe/Retractor B9 grasp the used Needle Carrier B7 and retract it as far as it is intended to be retracted. This must be done prior to any movement of the Rocker Arm C10 because, otherwise, the Probe/Retractor may not have had sufficient time to properly retract the used Needle Carrier. Once the Needle Carrier is retracted the Rocker Arm Dog C10D must engage a Cog B6A in the Cog Wheel B6 to advance the Cog Wheel, and thereby the Needle Carrier Wheel B8, the space of one Needle Carrier. The Elongated Slots C10E at the Hammer end of the Rocker Arm will allow the Hammer B4 to be partly cocked before the Rocker Arm Pin C10A reaches the end of the Elongated Holes C10E. By that time the proper Needle Carrier retraction must have been completed, and then the Wheel advancement may begin. The purpose of the Detent C11 is to keep the Wheel B8 from reversing its rotation once it has been made. The exact length of Elongated Hole C10E, the Rocker Arm Axle Pin C10C, and the Detent C11 must be precise for the mechanisms to function as they should.

FIG. 133 View of the bottom of the Needle Carrier Wheel B8. This is the side which will rest against the Cog Wheel B6 of Alternate Device No. 2. The Needle Carrier blocking function for Alternate Device Number 2 is contained at each Needle Carrier Channel B7G. The blocking parts are integral parts of the Wheel. They are Flaps B7F which are pressed out of the way, and encompassing part of the Needle Carrier, when the Needle Carrier is beside them, but fold down, when a Needle Carrier is partly retracted, to the location they had when the Needle Carriers B7 were not yet in the Needle-Carrier Slots B7G. When the Needle Carrier has been partly retracted, the Blocking Flaps, part B7F will then be in a position which will block reuse of the used Needle Carrier, and Cover Lips B7E will still retain the Needle Carrier in its slot. This Blocking Flaps B7F and Cover Lips B7E system will lend itself to the Needle Carriers being loaded into the Needle-Carrier Wheel by being pressed in laterally to the Slots B7G, rather than longitudinally, probably a more easily conducted procedure.

FIG. 134 Plan view of a single Needle Carrier of Alternate Device No. 2 in the Needle Carrier Wheel while in the position it would be in after having been used. The Blocking Flaps B7F have folded into a position at the Needle end of the Needle Carrier to disallow any further use of this Needle Carrier. The Cover Lips B7E will retain the Needle Carrier in its slot in the Wheel until the Needle Carrier Wheel is removed and discarded.

FIG. 135 A view at the Needle end of a used Needle Carrier of Alternate Device No. 2, showing the folded-down Blocking Flaps B7F.

FIG. 136 A view of a single Needle Carrier Slot B7G of Alternate Device No. 2 showing the Blocking Flaps B7F as well as the Cover Lips B7E

FIG. 137 Plan view of a single Needle Carrier B7 of Alternate Device No. 2 in the Needle Carrier Wheel B8 while in the position it would be in before it is used. The Blocking Flaps B7F partly cover the Needle Carrier. The Cover Lips B7E are also partly covering the Needle Carrier.

FIG. 138 A view at the Needle end of several of the Needle Carriers of Alternate Device Number 2 before they have been used. One Needle Carrier Slot B7G is empty to illustrate the unoccupied position of the Blocking Flaps and Cover Lips. The Blocking Flaps are in a folded-down position in their unoccupied state so they return to that condition when a Needle Carrier is not present.

FIG. 139 A section through a single Needle Carrier of Alternate Device Number 2 as it occupies its Slot B7G in the Needle Carrier Wheel of before it has been used.

FIG. 140 A section through the Needle Carrier Cap Connector looking towards the Needle Carrier body as used on Alternate Device Number 2.

FIG. 141 A section through the Needle Carrier body as used on Alternate Device Number 2.

FIG. 142 A view at the outer end of a Needle Carrier Retraction Cap as used on Alternate Device Number 2.

FIG. 143 A plan view of the top of the Cog Wheel B6 as used on Alternate Device Number 2. This is the side against which the Needle Carrier Wheel B8 will be placed.

FIG. 144 A side view of the Cog Wheel B6 as used on Alternate Device Number 2. The side shown is the side located at the Needle Aperture at the time a new Needle Carrier Wheel has been inserted but has not yet started on its rotational path.

FIG. 145 A section through the perimeter of the Cog Wheel B6 as used on Alternate Device Number 2.

FIG. 146 A plan view of the Hammer B4 as used on Alternate Device Number 2.

FIG. 147 A side view of the Hammer B4 as used on Alternate Device Number 2.

FIG. 148 A bottom view of the Hammer as used on Alternate Device Number 2.

FIG. 149 A face view of the Hammer as used on Alternate Device Number 2. The Cocking Lever B4B is an integral part of the Hammer B6.

FIG. 150 A view of the Dog C11A end of the Cog Wheel Detent C11 assembly as used on Alternate Device Number 2. The Cog Wheel Detent Assembly C11 has two functions. Primarily it keeps the Cog Wheel B6 from being dragged in reverse by the action of the Rocker Arm Dog CLOD when the Cocking Lever C10 is pulled and the Rocker Arm Dog CLOD is moving in reverse against the Cog Wheel surfaces. Additionally, the forcible seating of the Dog of the Cog Wheel Detent Assembly C11 will help to firmly center and keep centered the new Needle Carrier in the correct position for being struck by the Probe/Retractor.

FIG. 151 A view of the Rocker Arm assembly C10 at its Dog CLOD end as used on Alternate Device Number 2.

FIG. 152 A face view of the Cog Wheel Detent C11 assembly as used on Alternate Device Number 2.

FIG. 153 A side view of the Cog Wheel Detent Assembly as used on Alternate Device Number 2.

FIG. 154 A face view of the Rocker Arm assembly as used on Alternate Device Number 2.

FIG. 155 A side view of the Rocker Arm C10 assembly as used on Alternate Device Number 2.

FIG. 156 A view from the end opposite the Dog end of the Cog Wheel Detent assembly as used on Alternate Device Number 2.

FIG. 157 A view of the Rocker Arm assembly from the end opposite its Dog end as used on Alternate Device Number 2.

FIG. 158 A view of a Needle Carrier B7 of Alternate Device Number 2 from the Needle B7A end.

FIG. 159 A view of the Probe/Retractor B9 of Alternate Device Number 2 from the Retractor Jaws B9C end.

FIG. 160 A face view of a Needle Carrier B7 of Alternate Device Number 2.

FIG. 161 A side view of a Needle Carrier B7 of Alternate Device Number 2.

FIG. 162 A face view of the Probe/Retractor B9 of Alternate Device Number 2.

FIG. 163 A side view of the Probe/Retractor B9 of Alternate Device Number 2.

FIG. 164 An end view of a Needle Carrier B7 of Alternate Device Number 2 from the Retractor Cap B7B end.

FIG. 165 A bottom view of the Probe/Retractor B9 of Alternate Device Number 2.

FIG. 166 A view of the Needle Carrier B7 and Probe/Retractor B9 of Alternate Device Number 2 with the Needle Carrier B7 in position to be used, and the Probe/Retractor B9 fully retracted and cocked, ready to fire.

FIG. 167 A view of the Needle Carrier B7 and Probe/Retractor B9 of Alternate Device Number 2 with the Needle Carrier B7 in the position for it to be used, and the Probe/Retractor B9 is moving towards the Needle Carrier B7 preparatory to striking it.

FIG. 168 A view of the Needle Carrier B7 and Probe/Retractor B9 of Alternate Device Number 2 with the Needle Carrier having been struck by the Probe/Retractor, moving the Needle Carrier towards the point where it can make a puncture in a person's skin.

FIG. 169 A view of the Needle Carrier B7 and Probe/Retractor B9 of Alternate Device Number 2 with the Needle Carrier being shown at the moment its Needle makes a puncture.

FIG. 170 A view of the Needle Carrier B6 and Probe/Retractor B9 of Alternate Device Number 2 with the Needle Carrier being partly retracted from its puncturing position. The Needle Carrier should have reached this position under pressure from the Rebound Spring 4B, which must be selected for that purpose. From this point in the retraction process the retraction will be caused by the manual cocking of the Hammer B4 by use of the Cocking Lever B4B.

FIG. 171A view of the Needle Carrier B6 and Probe/Retractor B9 of Alternate Device Number 2 with the Needle Carrier having been fully retracted. The Spreader B3A has just engaged the flexible arms of the Probe/Retractor B9, and will open up the Retractor Jaws B7B and the Needle Carrier's Retractor Cap B7B will be released. It is at this point that the Dog CLOD on the Rocker Arm C10 is to engage a Cog B6A in the Cog Wheel B6, and by the time the Hammer B4 is fully recocked the Cog Wheel B6, and thereby the attached Needle Carrier Wheel B7, should have advanced the space of one Needle position, and a new Needle Carrier should have been moved into the position preparatory to its firing.

FIG. 172 A view of the Needle Carrier B7 and Probe/Retractor B9 of Alternate Device Number 2 with the Needle Carrier now fully released. The Blocking Flaps B7F are now engaged to disallow further use of this Needle Carrier. The Probe/Retractor B9 is still moving under force from the Cocking Lever B4B, and will quickly be in a cocked position again.

FIG. 173 A view of the Needle Carrier B7 and Probe/Retractor B9 of Alternate Device Number 2 with a new Needle Carrier now in position to be used, and the Probe/Retractor fully retracted and cocked, ready to fire.

FIG. 174 A longitudinal section of Alternate Device Number 3 with the various part numbers shown. Many of the parts are the same as those found on Alternate Device Number 1 and Alternate Device Number 2. The Needle Carriers C6, Needle-Carrier Wheel C8, Probe Retractor C7, and Hammer C9 all have some minor changes from comparable parts from Alternate Device Number 1 and Alternate Device Number 2. The changes were made necessary because of the changes in the Needle Carriers, namely, the oval section and Stop Arm C6B shapes on Needle Carrier C6.

FIG. 175 This is the same as FIG. 174, except that no parts numbers are shown. This is done to enable a clearer vision of the device's parts. In this view the Hammer C9 is cocked and the Needle Carrier C6 is new and ready to be impelled into the target's skin.

FIG. 176 Same as FIG. 175. Placed alongside FIG. 177 as an aid in understanding the information in FIG. 177.

FIG. 177 A plan view of Alternate Device Number 3 with its Case Lid B2 removed.

FIG. 178 A view of the bottom of the Needle-Carrier Wheel C8 of Alternative Device Number 3 as seen from the side which will rest against the Cog Wheel B6.

FIG. 179 A view of part of the outer edge of Needle-Carrier Wheel C8 showing the oval-shaped Needle-Carrier Slots C8A. In order for the matching deformations on the Probe/Retractor Jaws and the Needle-Carrier Caps to be kept in alignment, the Needle Carrier must not twist, so the Needle Carriers C6 and the Needle-Carrier Slots C8A are being made in an oval shape. This will preclude twisting of the Needle Carriers as they might do if made cylindrically. However, if there is some advantage in their manufacture if a rectangular section replaces the oval section, that would work as well. Further, the reduced lateral dimension made possible by an oval or rectangular shape will allow more Needle Carriers to be placed in the Wheel than would cylindrical Needle Carriers. The Needle Carriers, whichever type is used in the final production of this device or any of the devices shown in the present invention, are to be made as small in lateral dimension as is possible, consistent with their structural integrity during actual usage, for this will permit the use of the greatest number of Needle Carriers in the present invention. The Stop Arms C6B of the Needle Carriers will be formed, when they are manufactured, spread out from the body of the Needle Carrier. These will need to be depressed all the way to the Needle-Carrier C6 body in order to insert them into the Needle-Carrier Wheel C8 when the Needle Carriers are being loaded into the Wheel, and they will remain tightly alongside the Needle-Carrier body until such time as they are partially retracted, at which time the Stop Arms C6B will spring back to their natural position, Will catch on the Needle-Carrier Wheel's edge, keeping the Needle Carriers from reentry into their slots, thereby preventing any accidental reuse of the Needle Carrier. The Stop Arms must be made as small as is possible, consistent with their being able to not be crumpled if called on to resist a blow from the Hammer. All of these parts may be made of plastic or metal.

FIG. 180 This shows a section through one of the oval Needle Carriers C6 of Alternative Device Number 3 while it is in a firing position inside its Needle-Carrier Wheel C8.

FIG. 181 In this view the Hammer C9 and Probe/Retractor C7 of Alternative Device Number 3 are cocked and ready to be fired. A new Needle Carrier C6 is in the proper position to be propelled into a target's skin by the Probe/Retractor C7.

FIG. 182 In this view the Hammer C9 of Alternative Device Number 3 has been released by the Pushbutton 5 and the Probe/Retractor Jaws C7C have bumped the Cap C6E of the Needle Carrier C6. The Jaws have slid over the Cap, moving it a bit. Now the Shoulders of the Jaws will strike the Cap and move the Needle C6A toward the target's skin.

FIG. 183 In this view of Alternative Device Number 3 the Jaws C7C have completely clamped onto the Cap C6E of the Needle Carrier C6, and the Needle Carrier is well on its way to making a puncture in the target's skin, if any is present.

FIG. 184 This is a view of the Alternate Device Number 3 at the moment its Needle C6A is making a puncture. The Hammer B4 has been stopped by the Spreader Bar B3A. The Needle Carrier has been stopped by its rim C6C, which has met the Needle-Carrier Wheel's C8 inner edge.

FIG. 185 This is a view of the Alternate Device Number 3 at the time the Jaws are pulling the Needle Carrier back from a puncturing position, and the spreader bar has just met the Probe/Retractor Arms C7B which are about to be spread so the Jaws can release the Needle-Carrier Cap, thus completing the retraction of the used Needle Carrier. Immediately thereafter the Rocker Arm C10 will, during the remainder of the travel left in the Rocker Arm, engage the striking side of a Cog B6D and will advance the Needle-Carrier Wheel to position a new Needle Carrier to a usable location.

FIG. 186 In this view the Needle Carrier C6 has been retracted far enough so two wings C6B on the Needle Carrier have popped out from the Needle-Carrier body. These Wings will keep the used Needle Carrier from being accidentally reused. In actual practice momentum may have carried the Needle Carrier a bit further, but it will remain in its slot, and cannot be used again. The Rocker Arm is just now beginning to rotate the Needle-Carrier Wheel.

FIG. 187 This view concludes the series of drawings showing Needle-Carrier movement from start to finish. Here, a new, unused Needle-Carrier C6 has been moved into position, ready to be impelled. The Hammer C9 has been cocked and is ready for firing.

FIG. 188 A face view of Needle Carrier C6 for use in Alternate Device Number 3. The Stop Arms C6B, Needle C6A, Stop Rim C6C, Cap Connector C6D, and Cap C6E are shown. Because of the shape of the Stop Arms an oval-sectioned body will work best for this Needle Carrier. The Cap is deformed to fit complementary deformations in the Jaws C7C of Probe/Retractor C7 used with this Needle Carrier.

FIG. 189 A side view of Needle Carrier C6.

FIG. 190 A view of the Cap C6E on Needle Carrier C6 from the end opposite the Needle end.

FIG. 191 A view of the Needle end of Needle Carrier C6.

FIG. 192 A view of the Needle end of Needle Carrier C6.

FIG. 193 A face view of Probe/Retractor C7 of Alternate Device Number 3.

FIG. 194 A side view of Probe/Retractor C7.

FIG. 195 A view of the end of Probe Retractor C7 at the end opposite the Jaws C7C.

FIG. 196 A view of Hammer C9 from the end where the Probe/Retractor C7 will be socketed on Alternate Device Number 3.

FIG. 197 A side view of Hammer C9. A Spreader Recess C9A is shown, as is a Probe/Retractor Socket C9B. The Cocking Lever B4B is shown.

FIG. 198 A side view of Hammer C9.

FIG. 199 A view of Hammer C9 from the Cocking Lever end.

FIG. 200 A view of a portion of Alternate Device Number 3 showing the use of a rimless Needle Carrier C12 and a Cog Wheel C13 which incorporates a bumper rim C13B used to limit the forward travel of the now rimless Needle Carrier.

FIG. 201 Shows the needle-end view of rimless Needle Carrier C12.

FIG. 202 A face view of rimless Needle Carrier C12.

FIG. 203 A side view of rimless Needle Carrier C12.

FIG. 204 A view of rimless Needle Carrier C12 from its cap end.

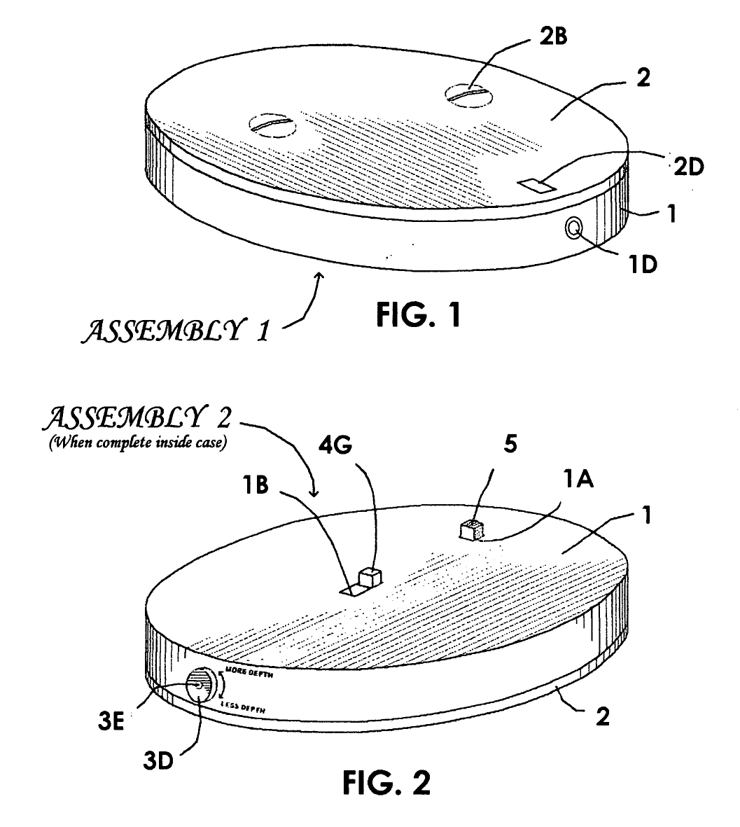

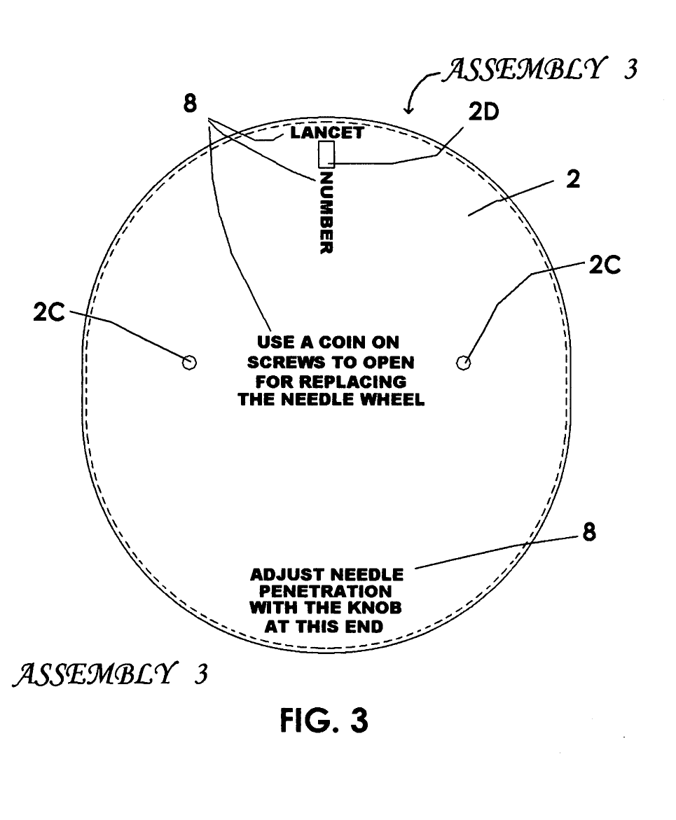

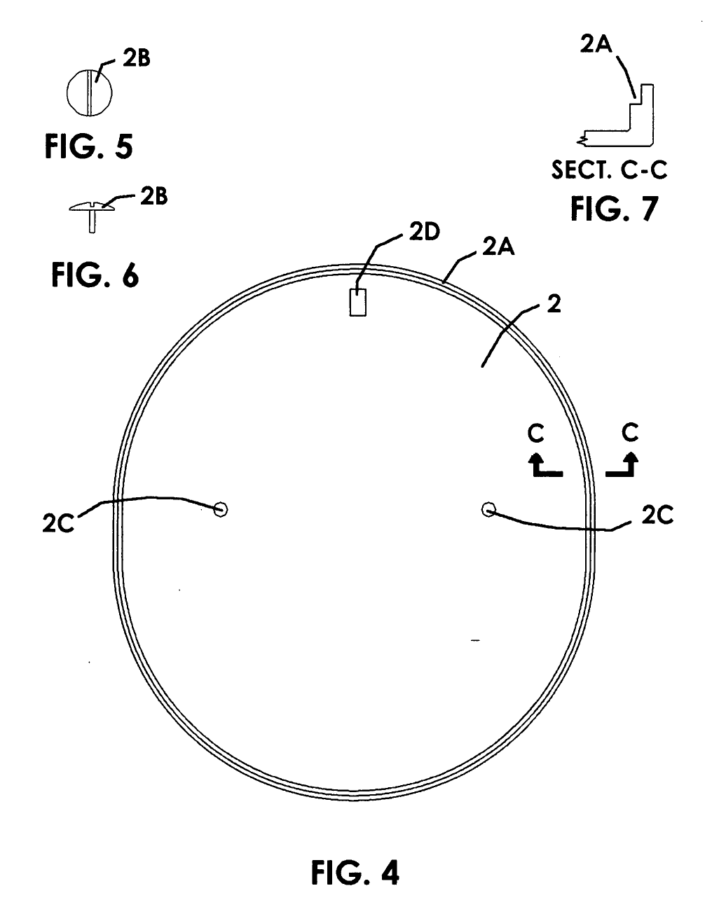

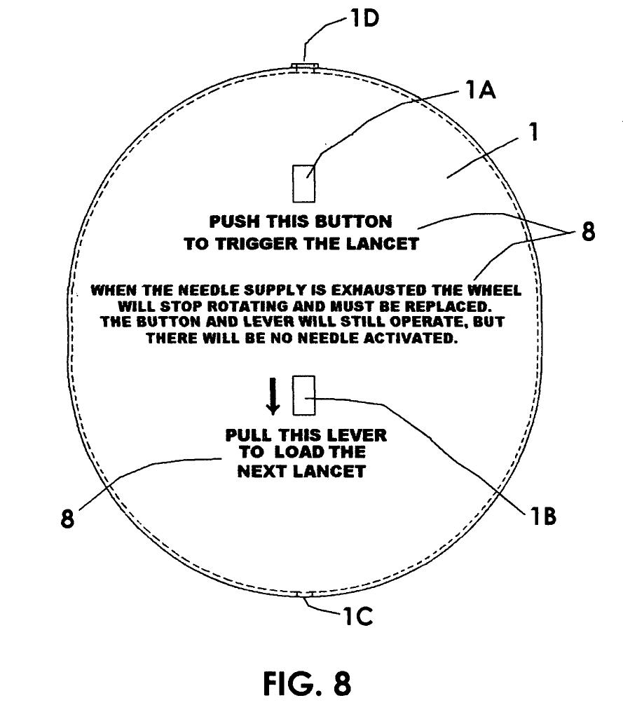

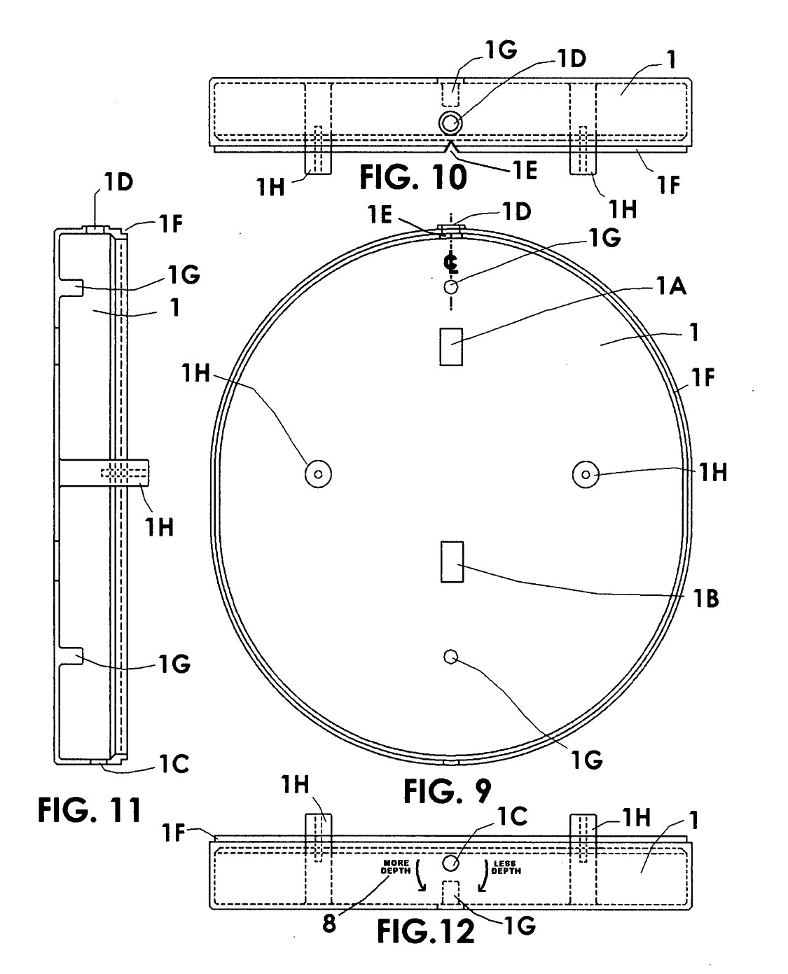

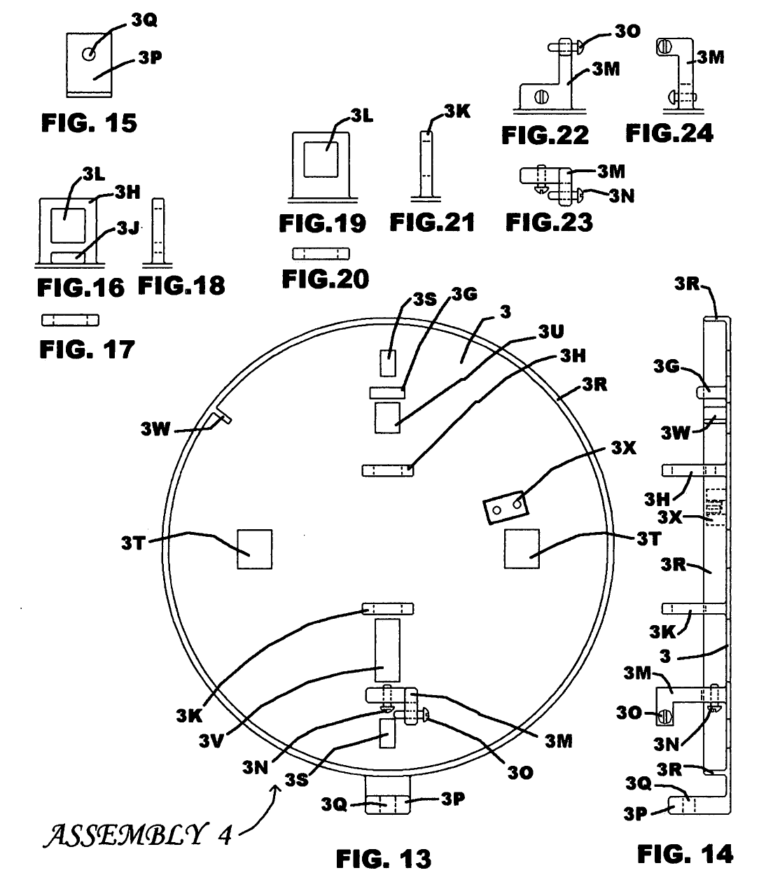

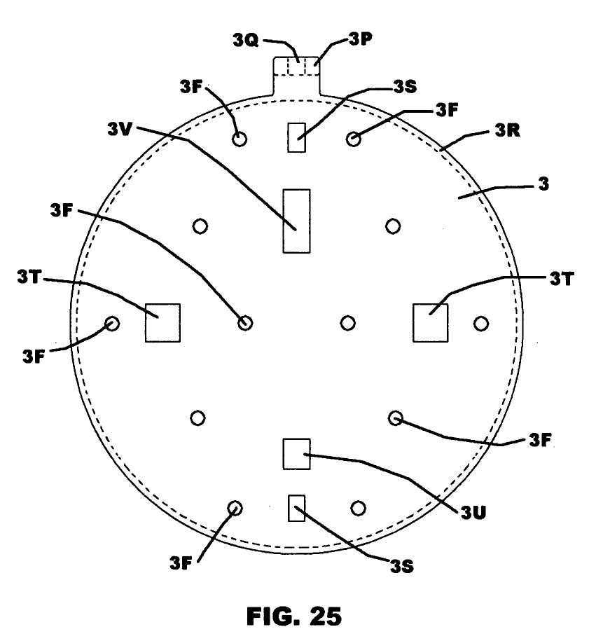

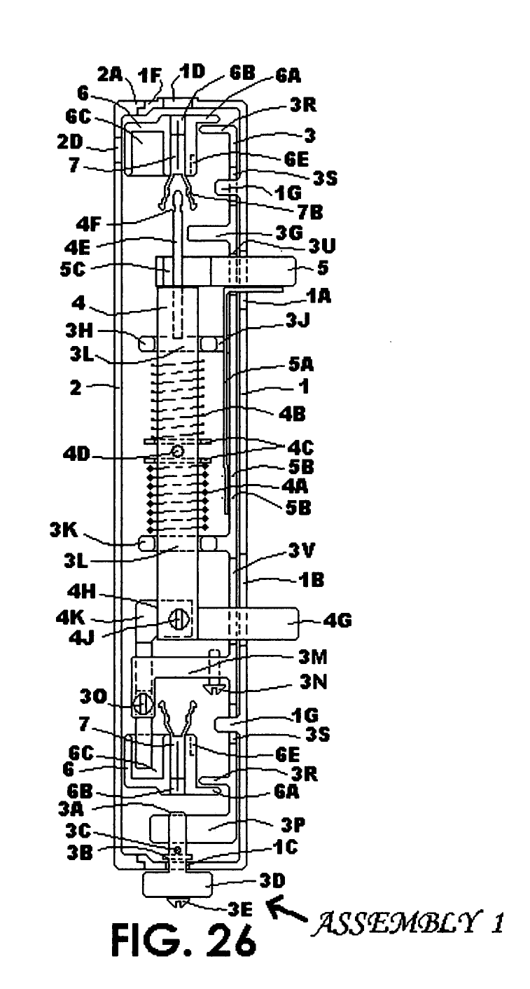

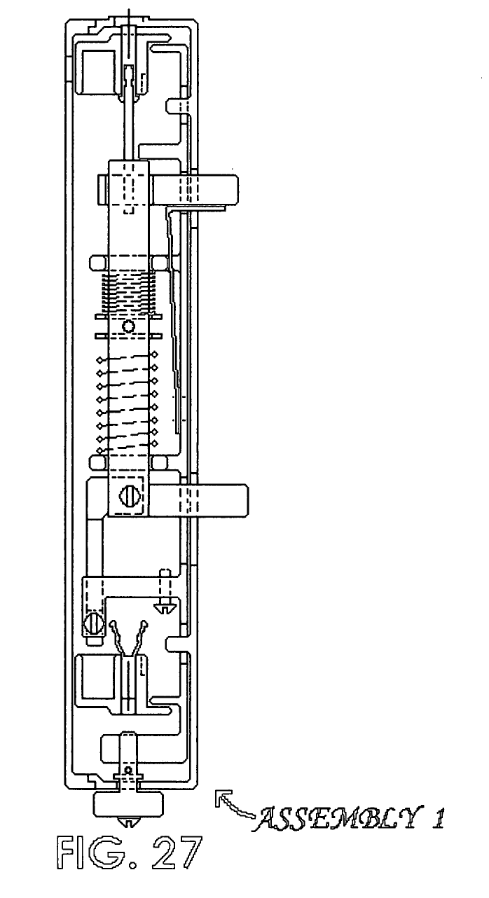

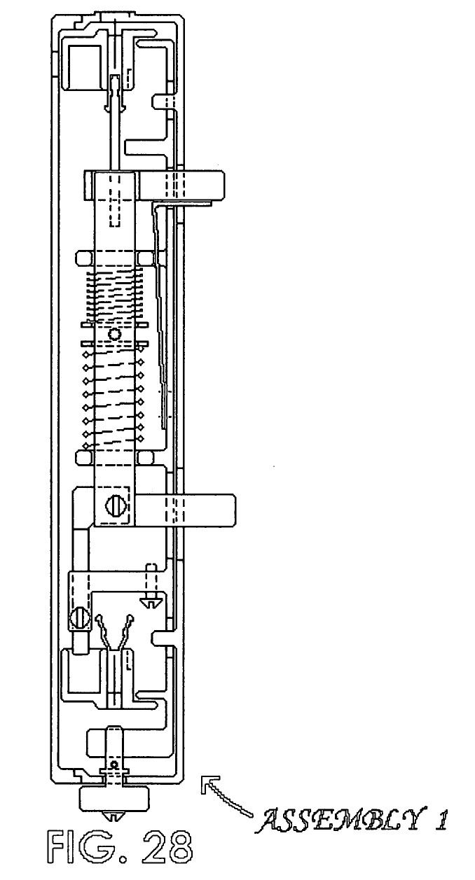

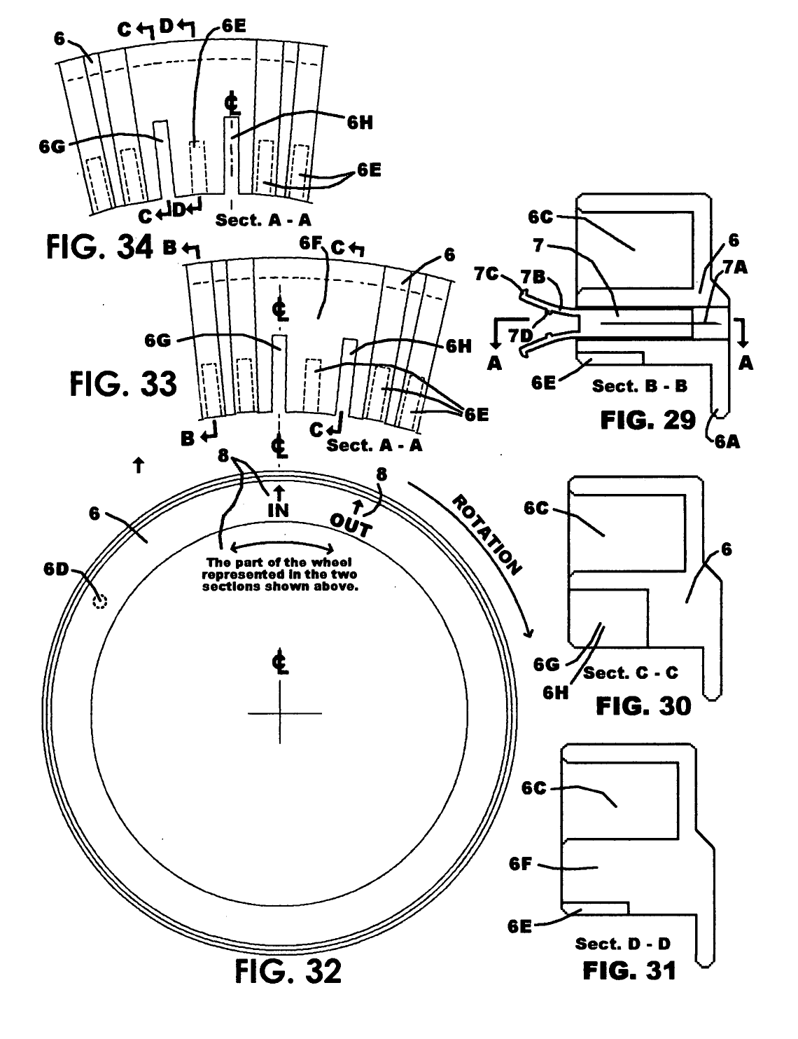

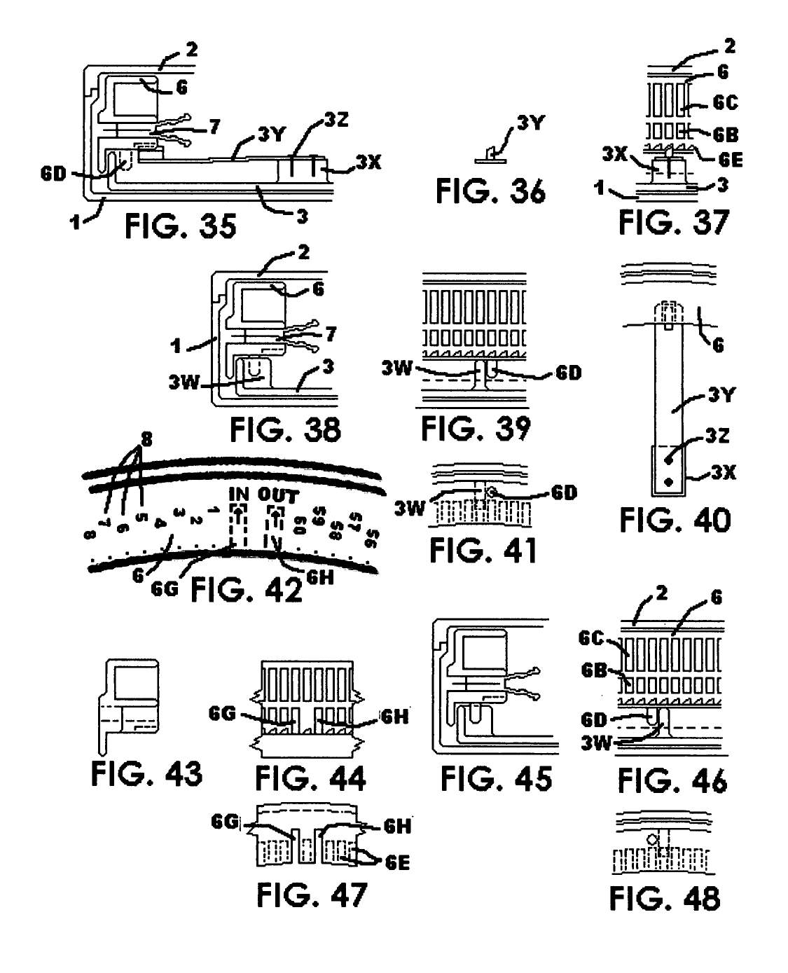

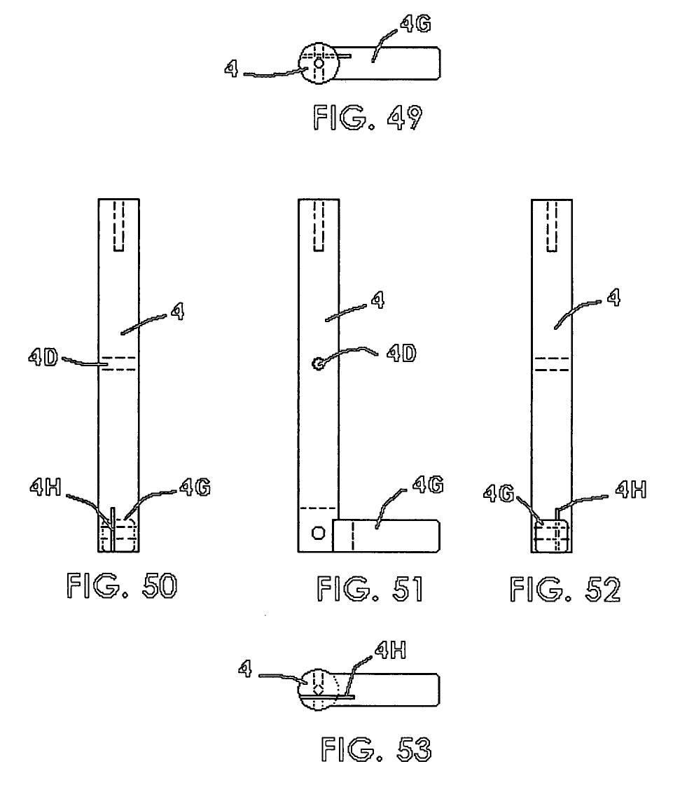

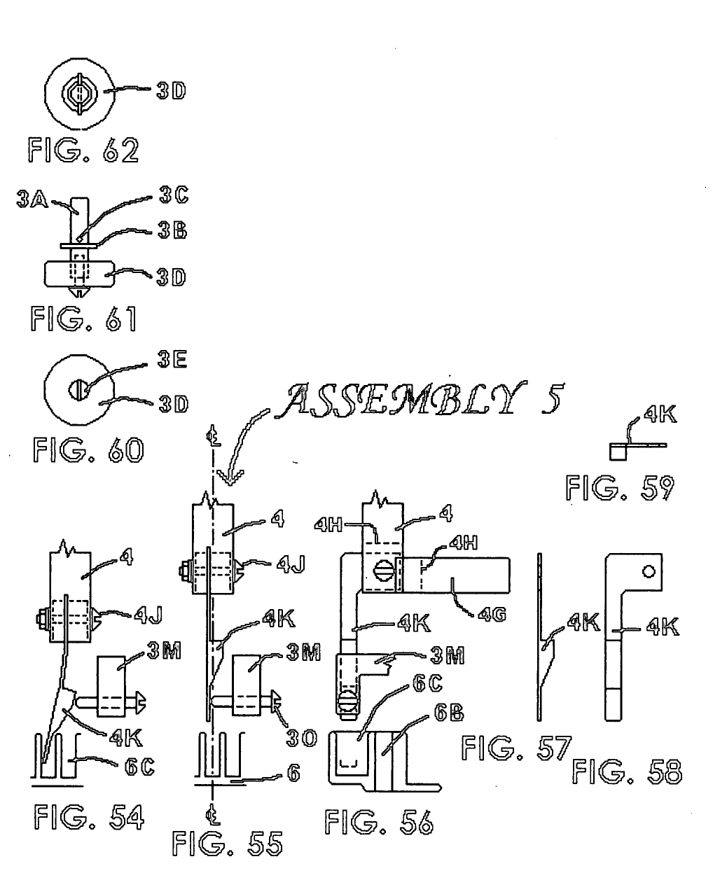

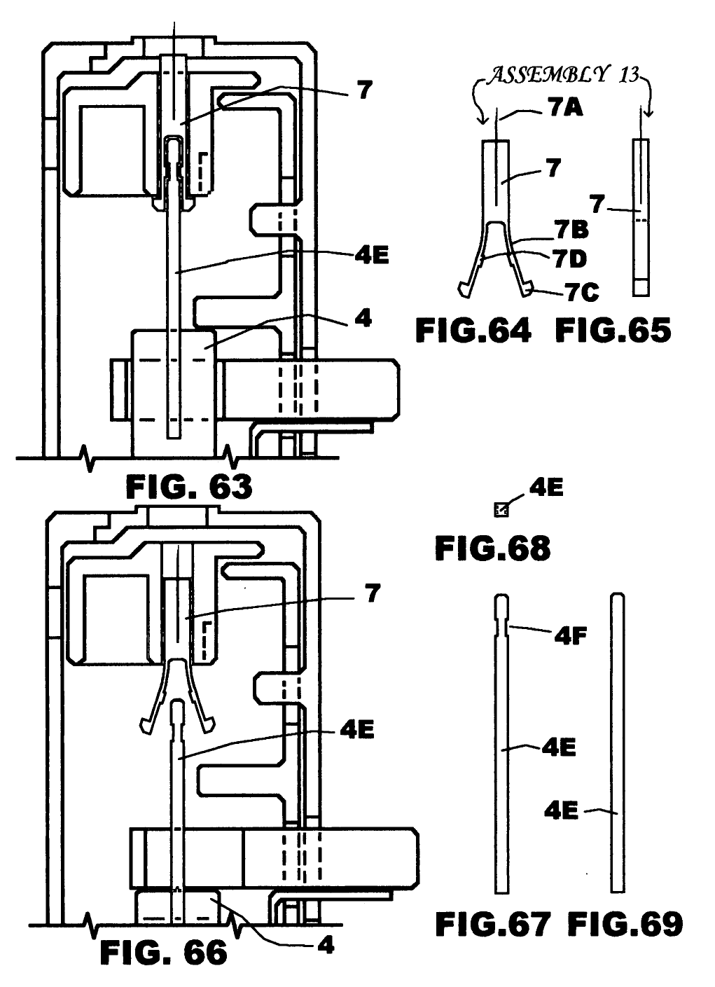

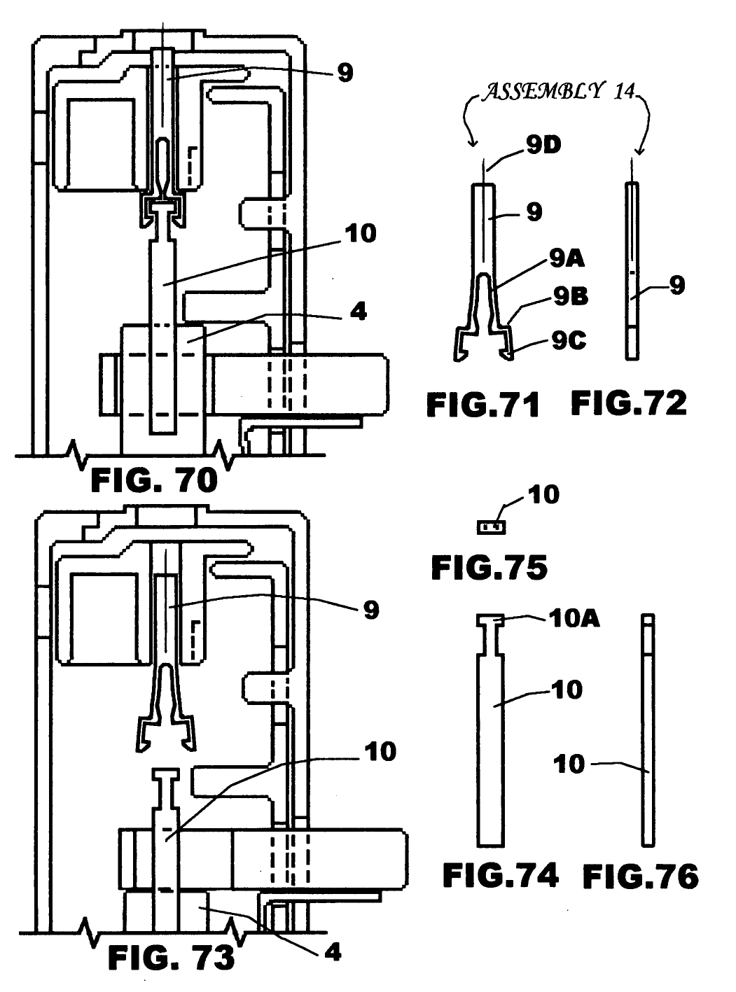

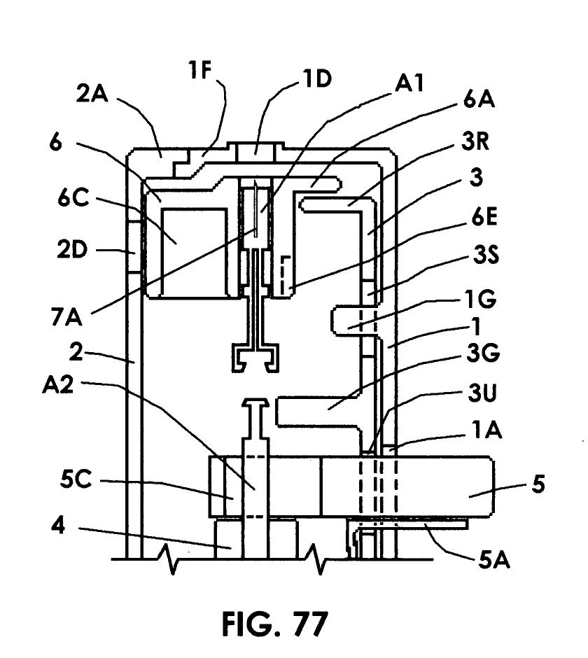

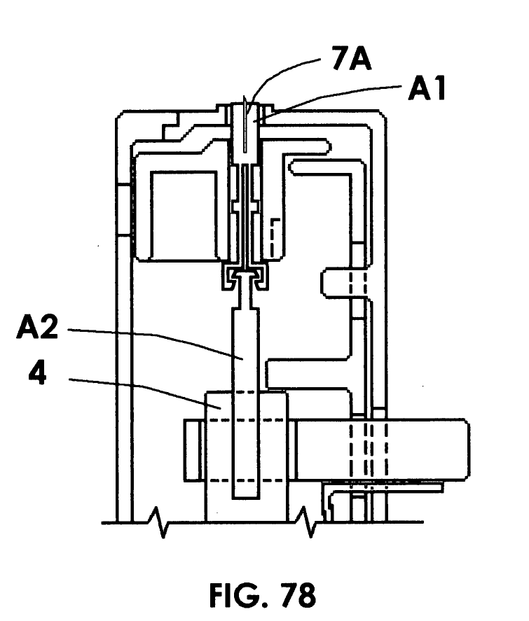

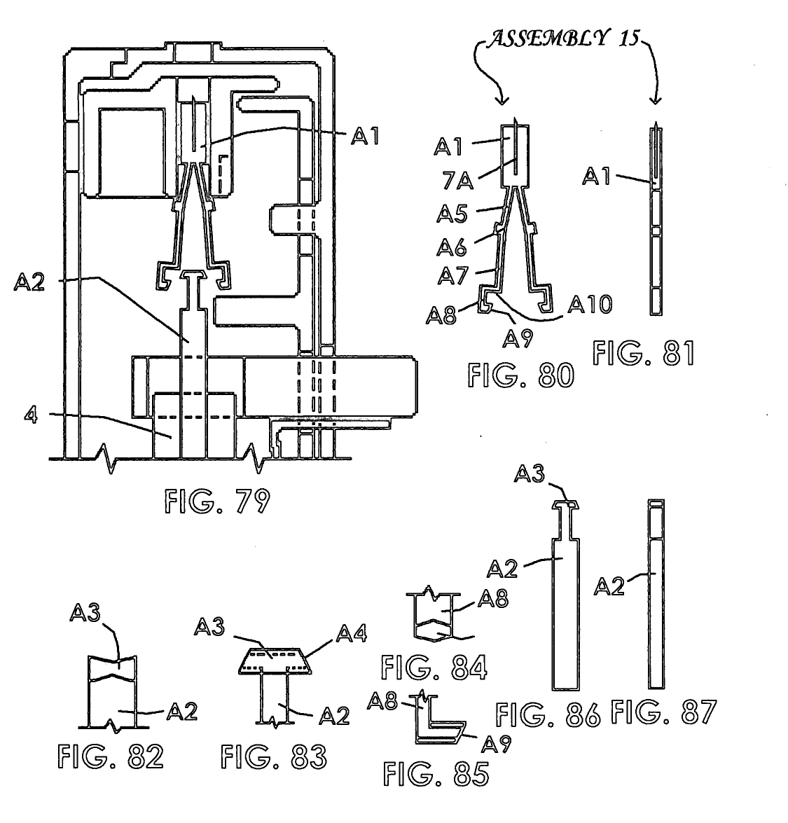

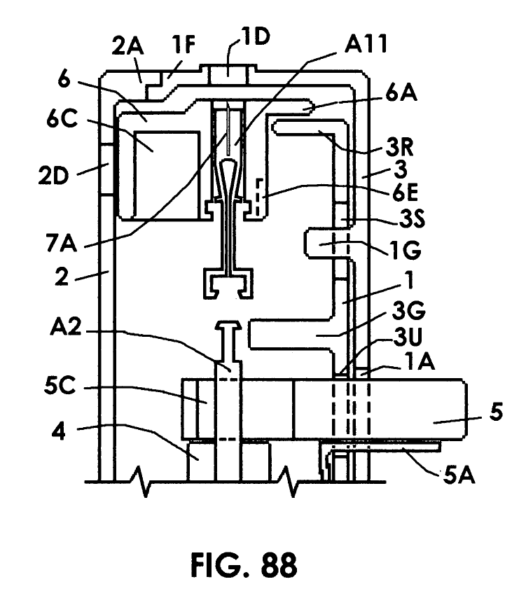

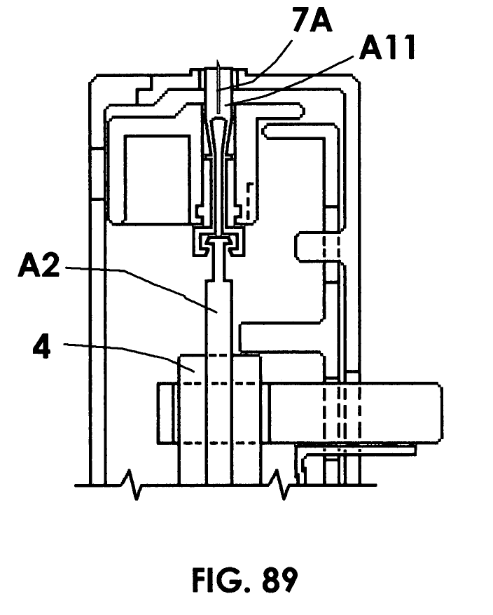

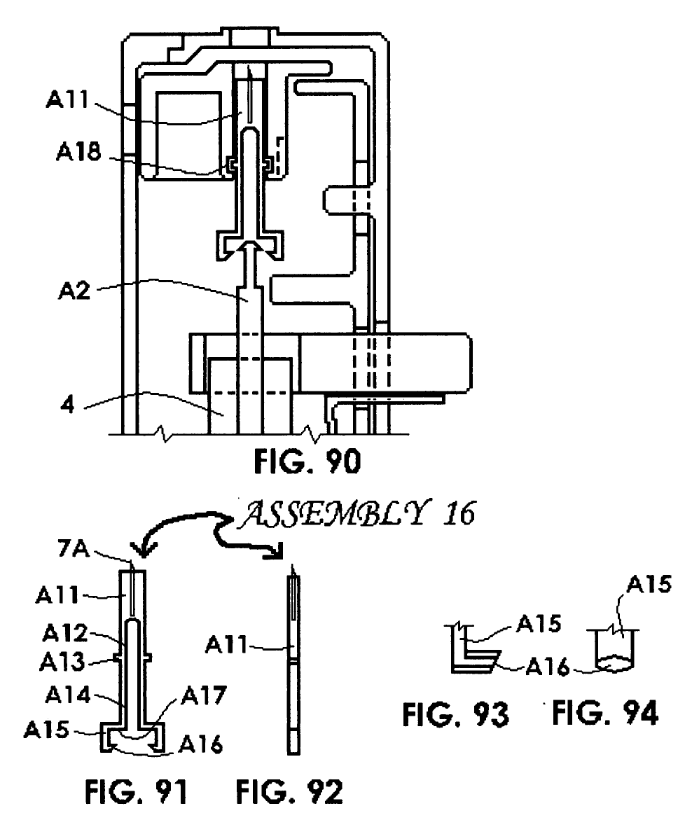

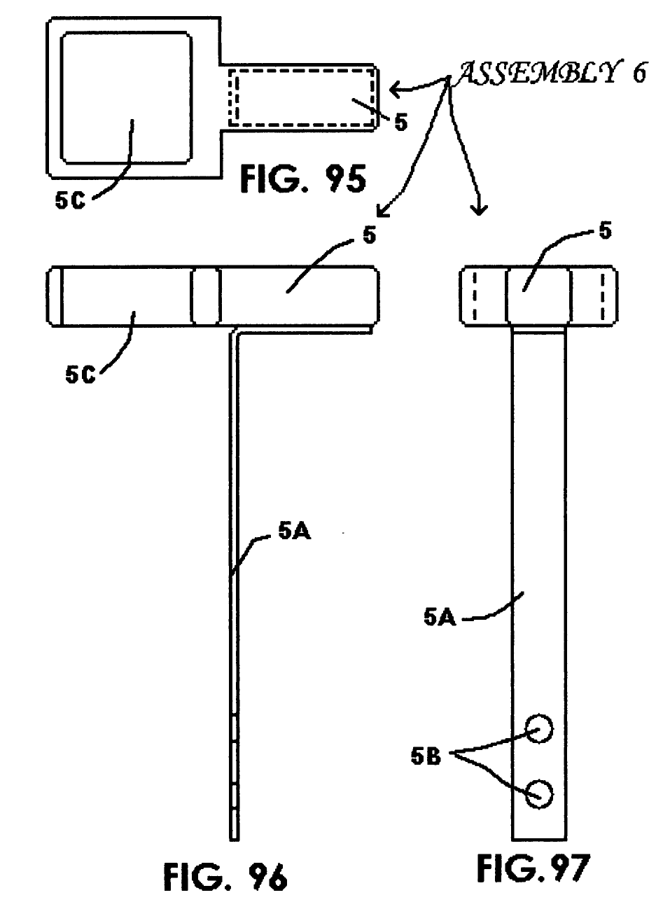

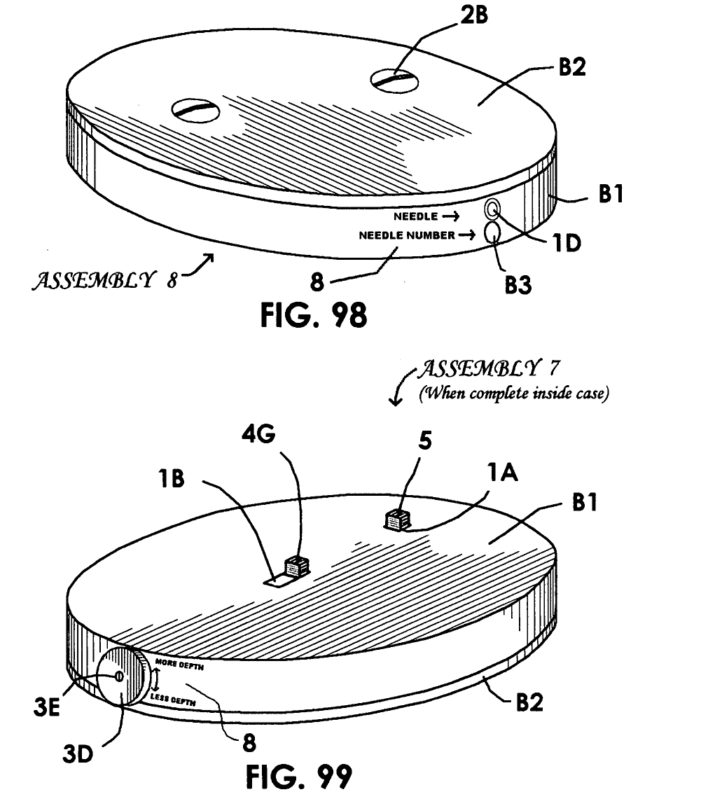

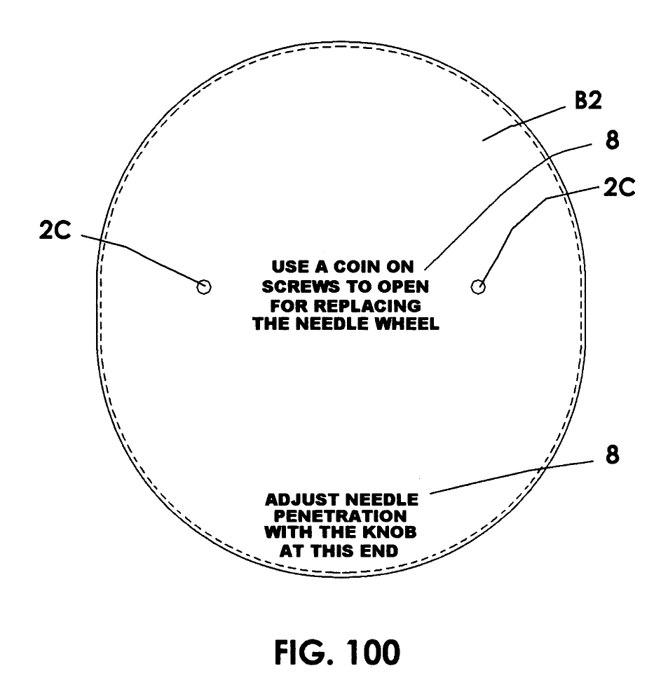

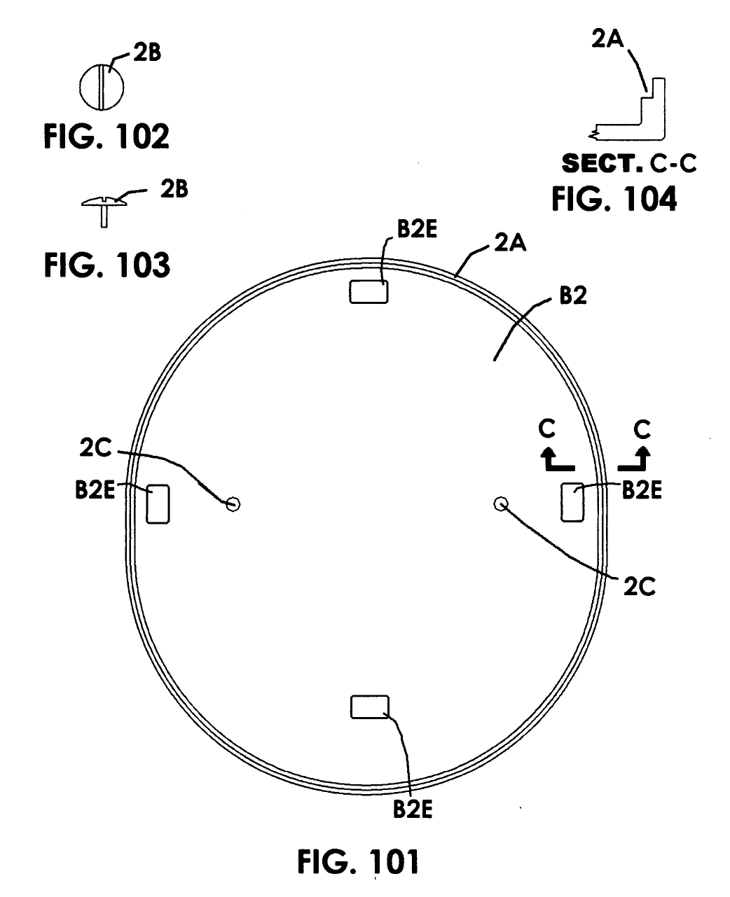

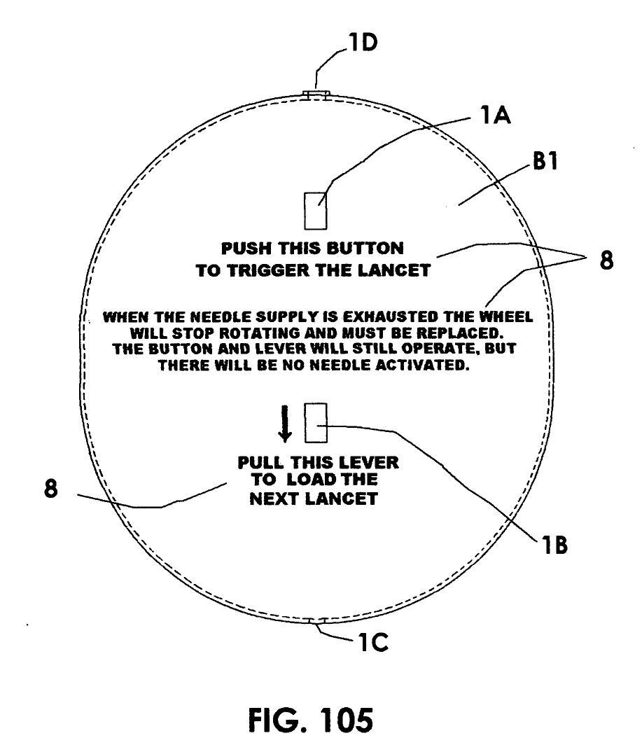

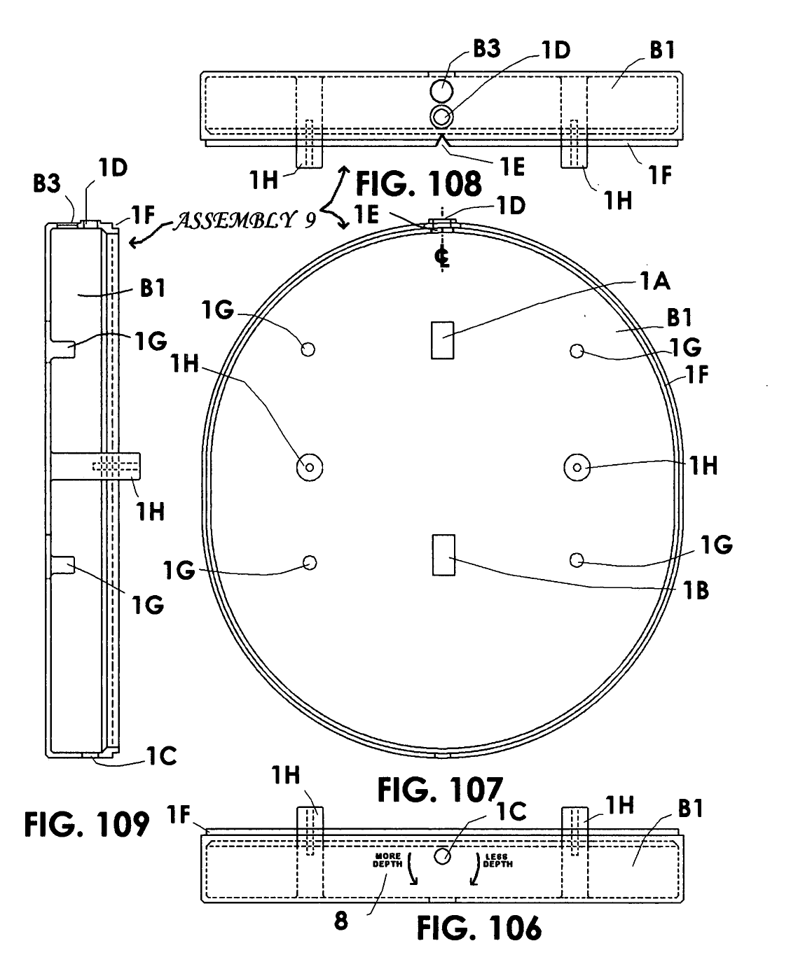

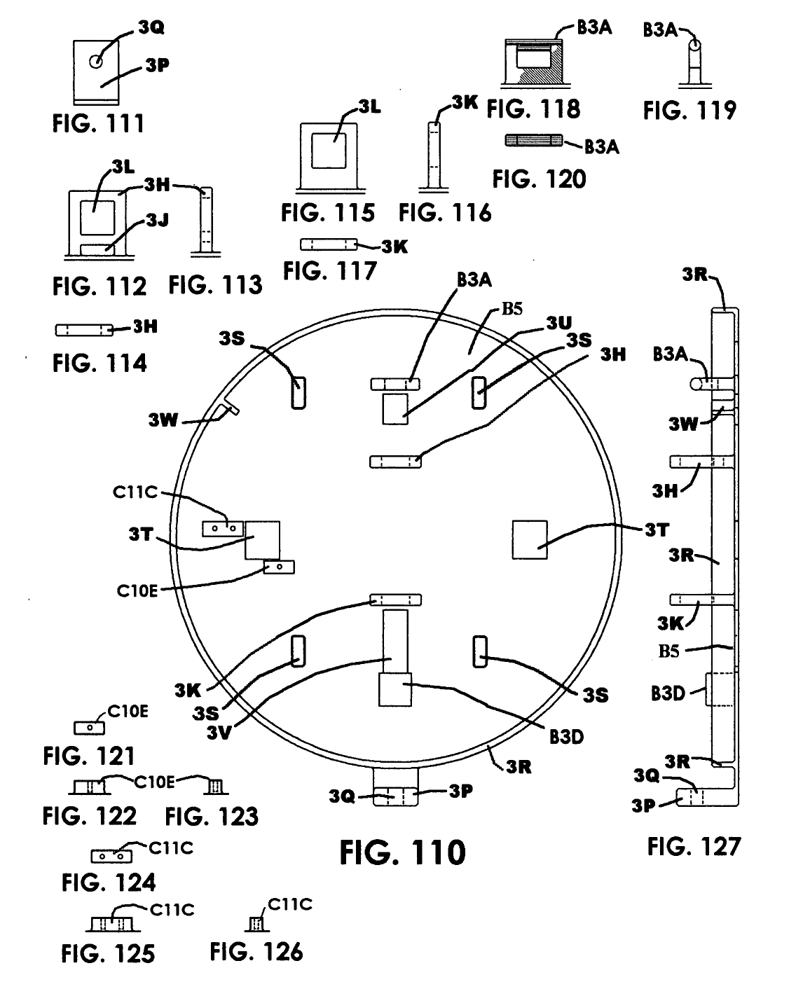

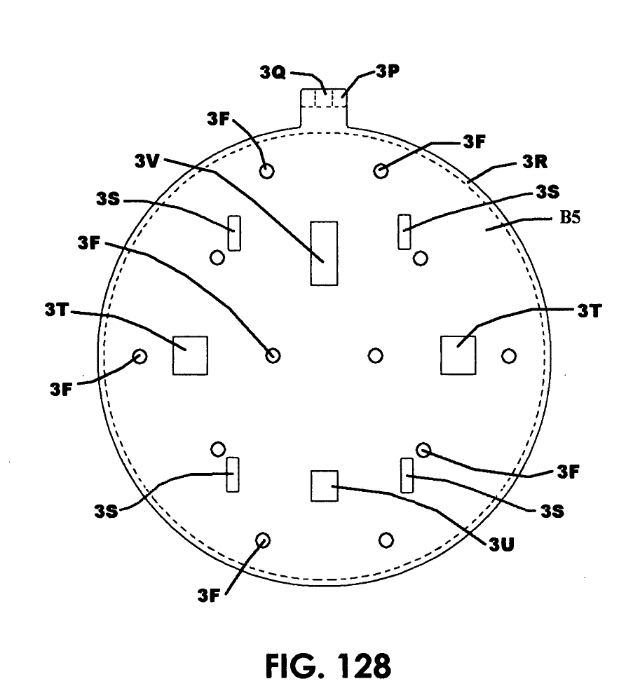

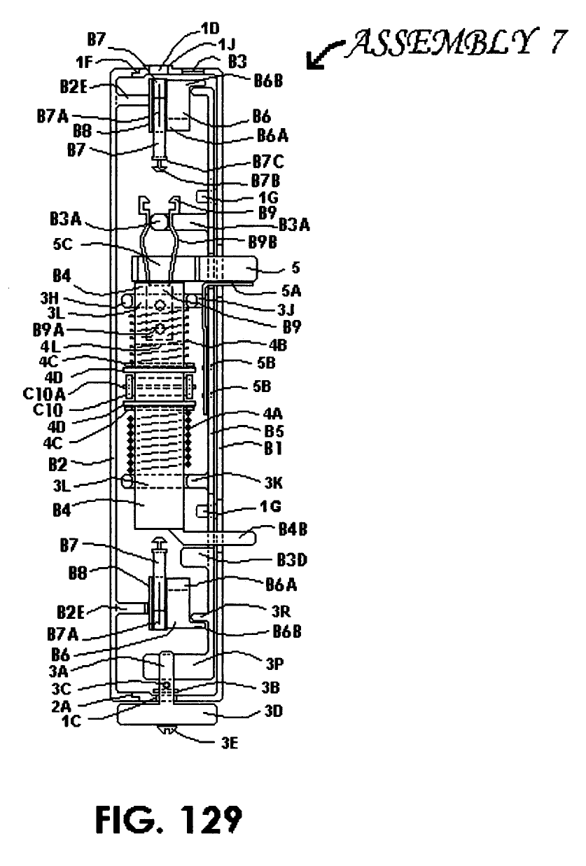

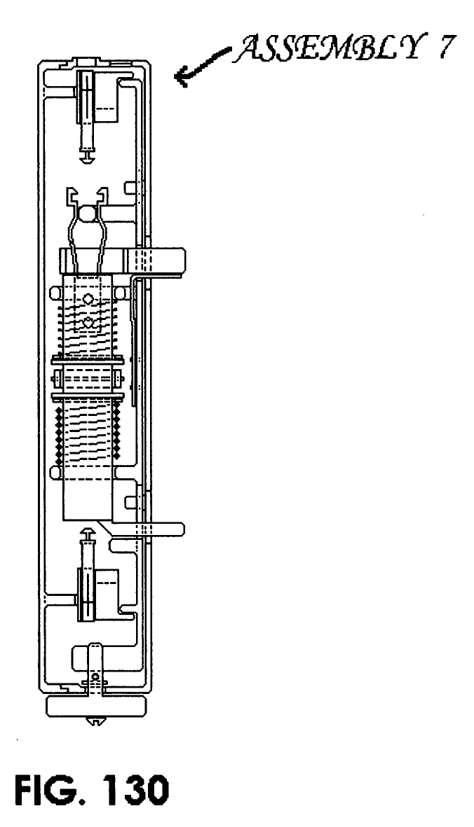

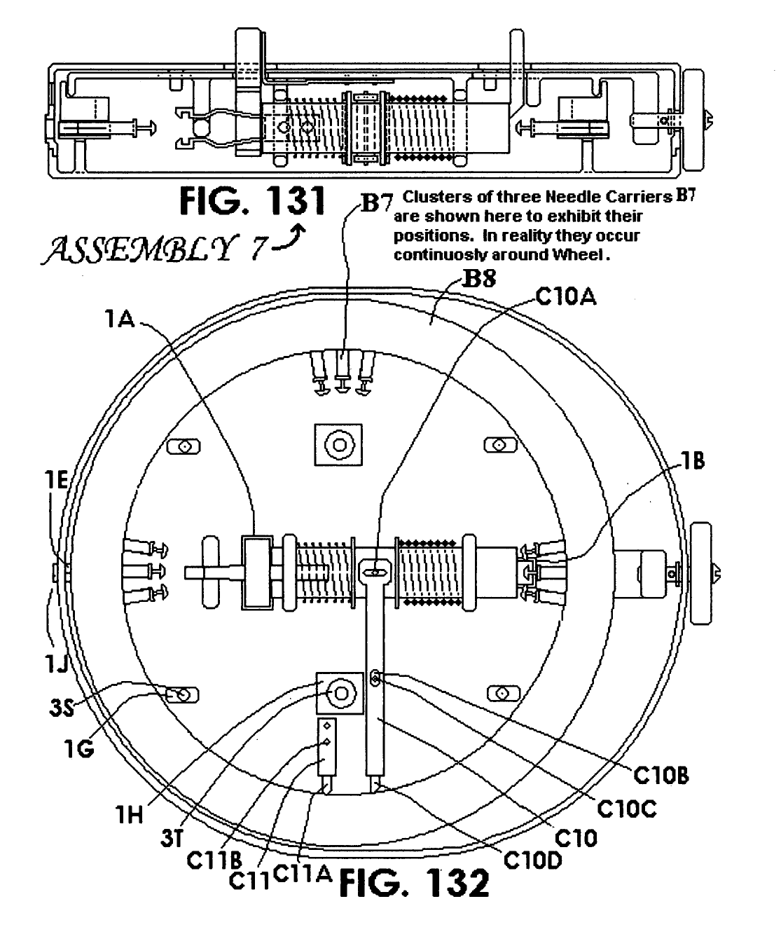

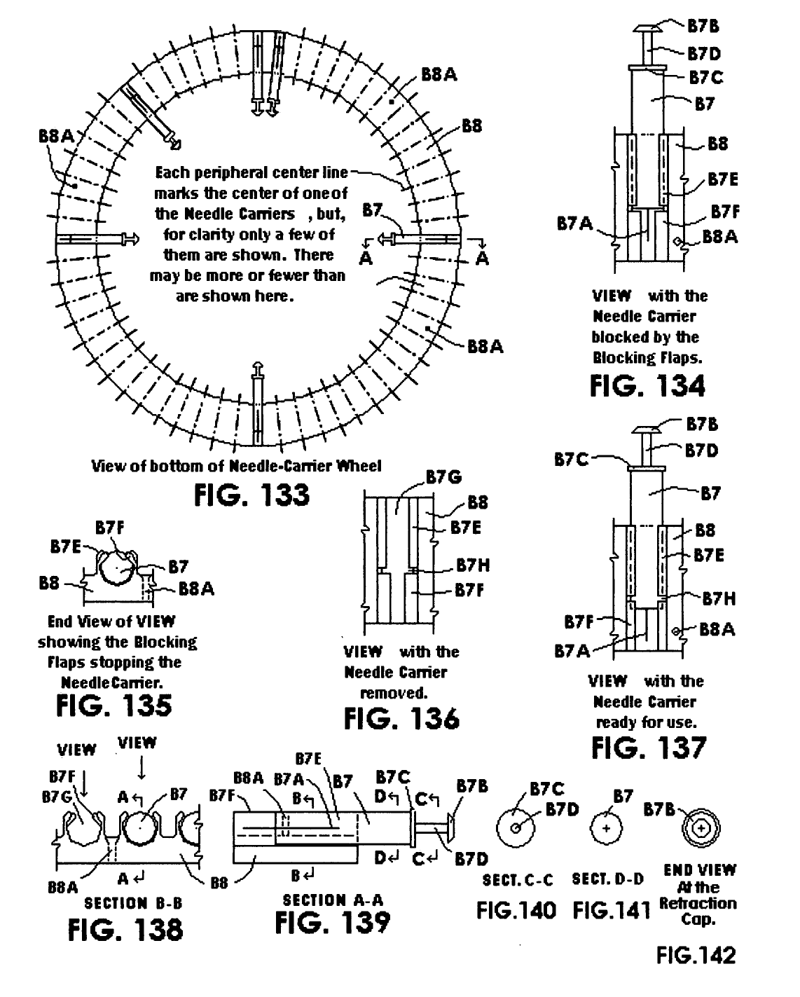

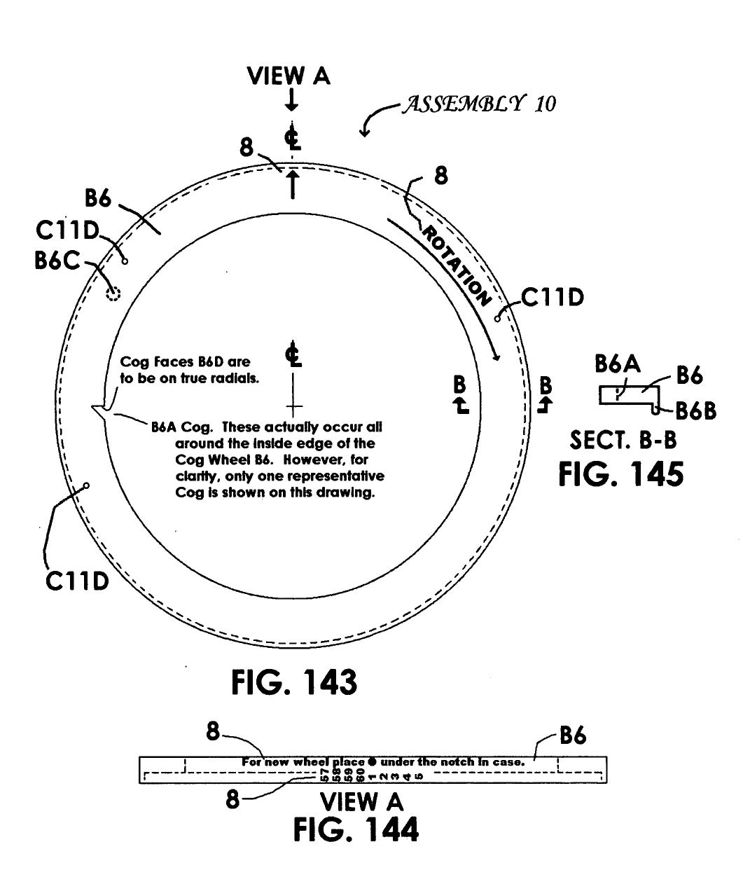

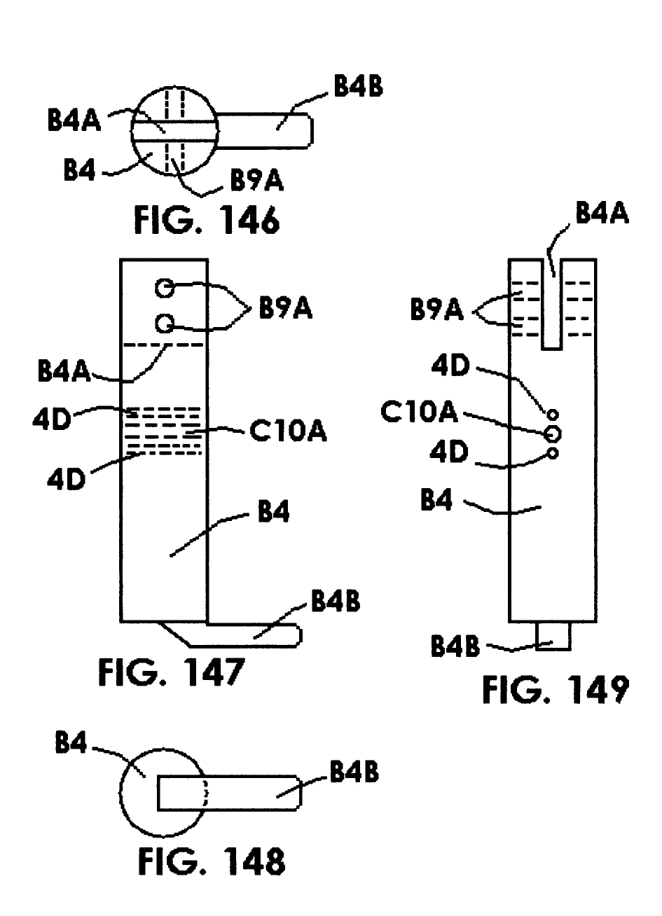

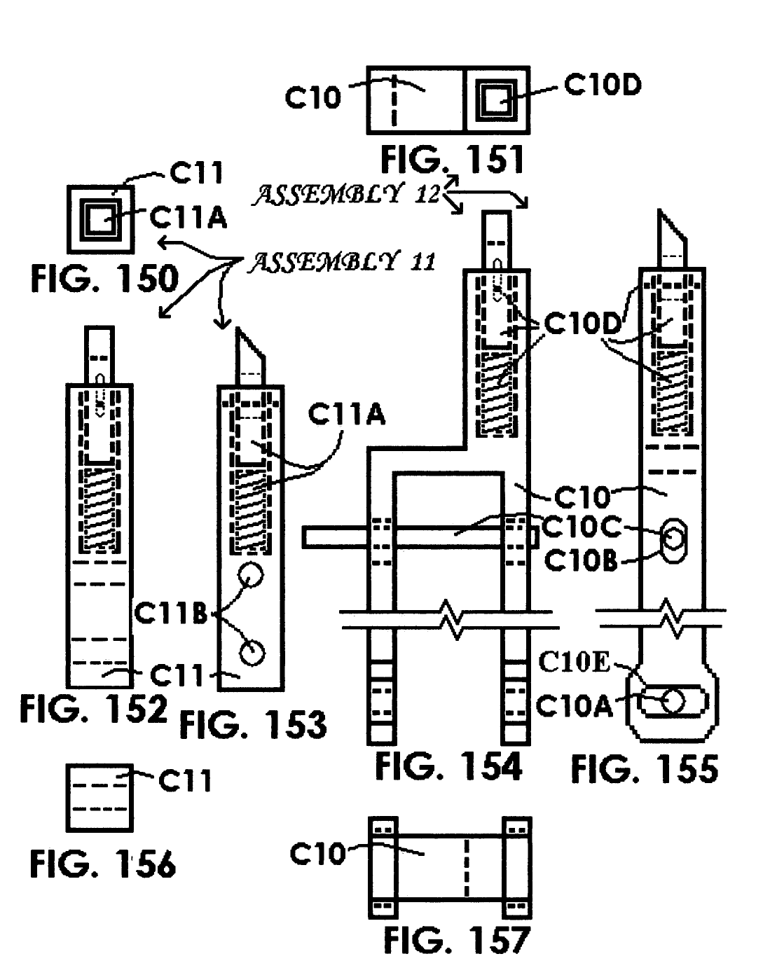

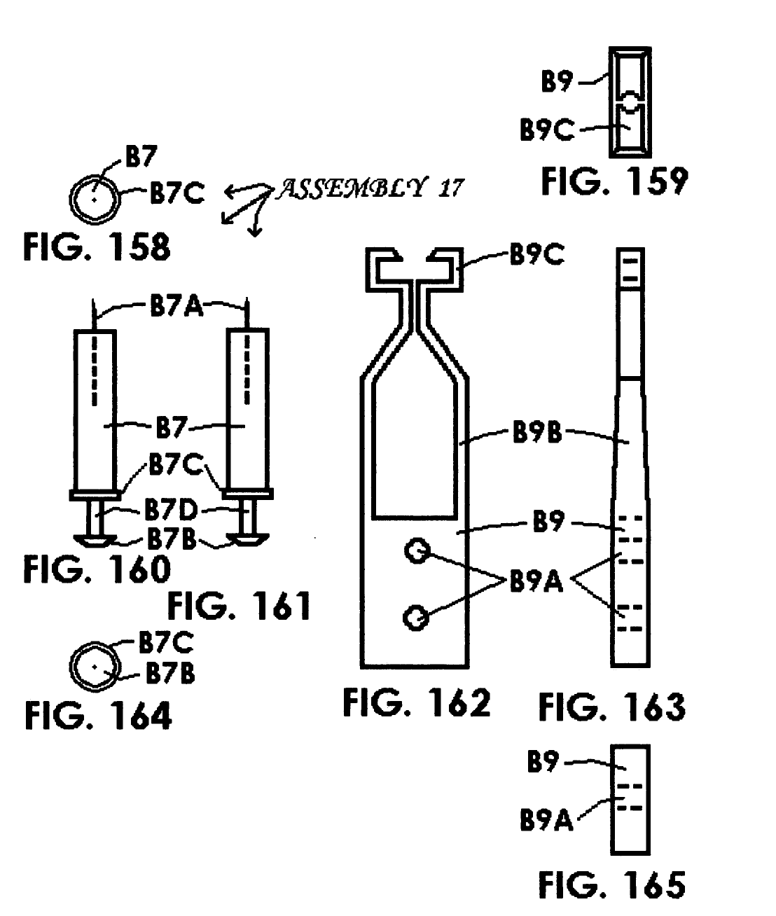

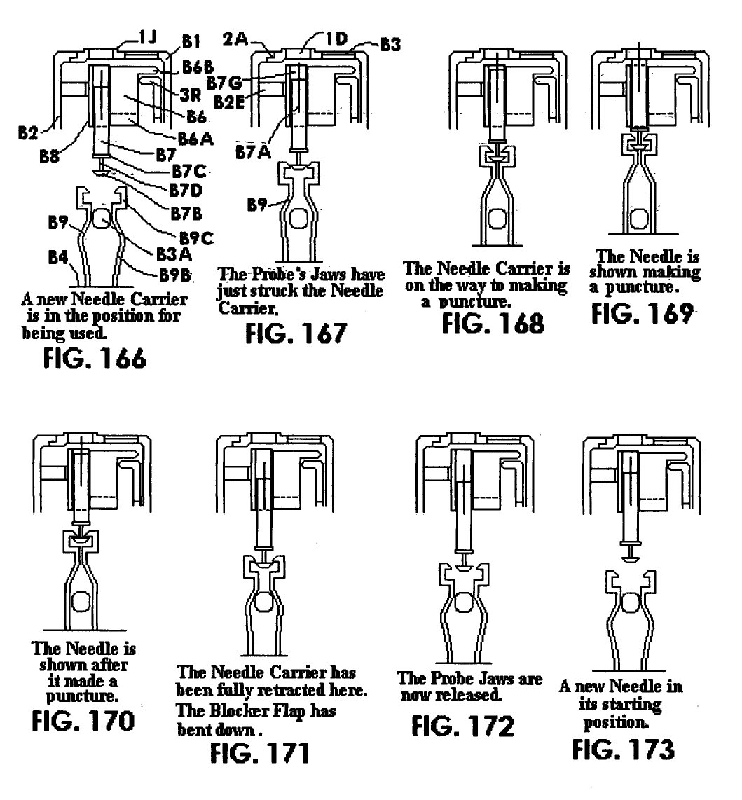

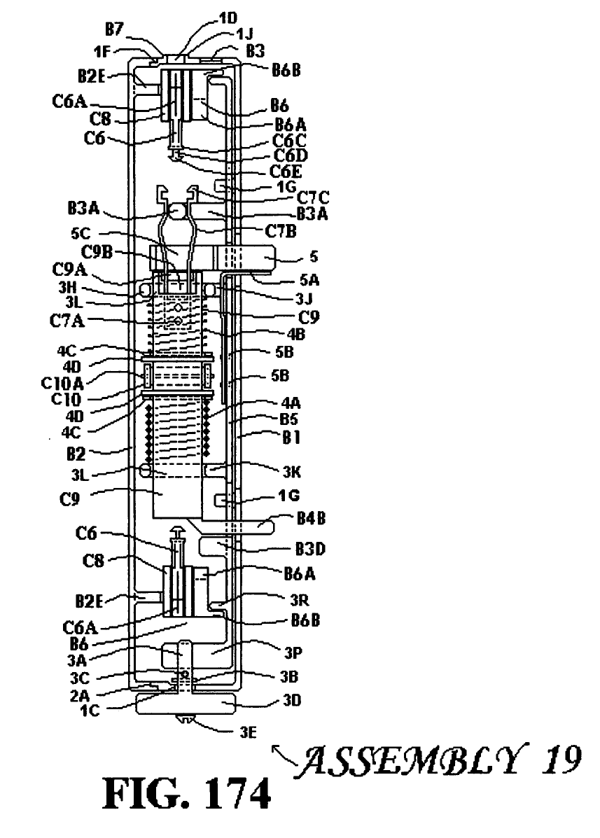

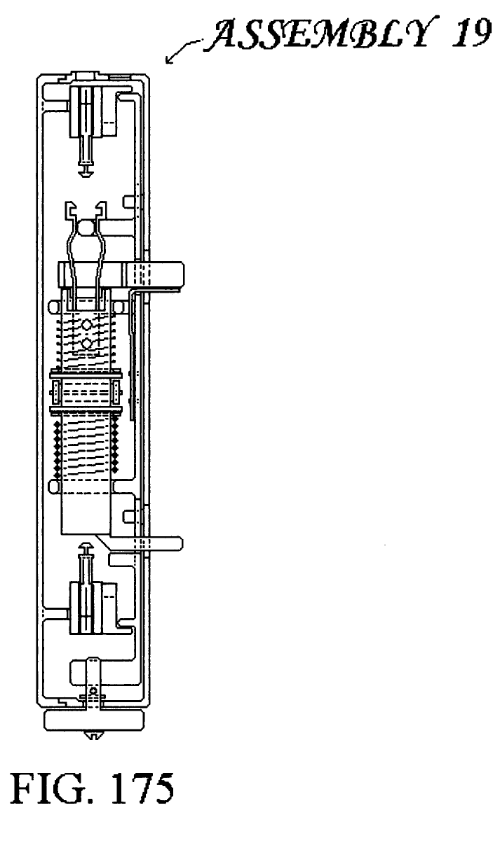

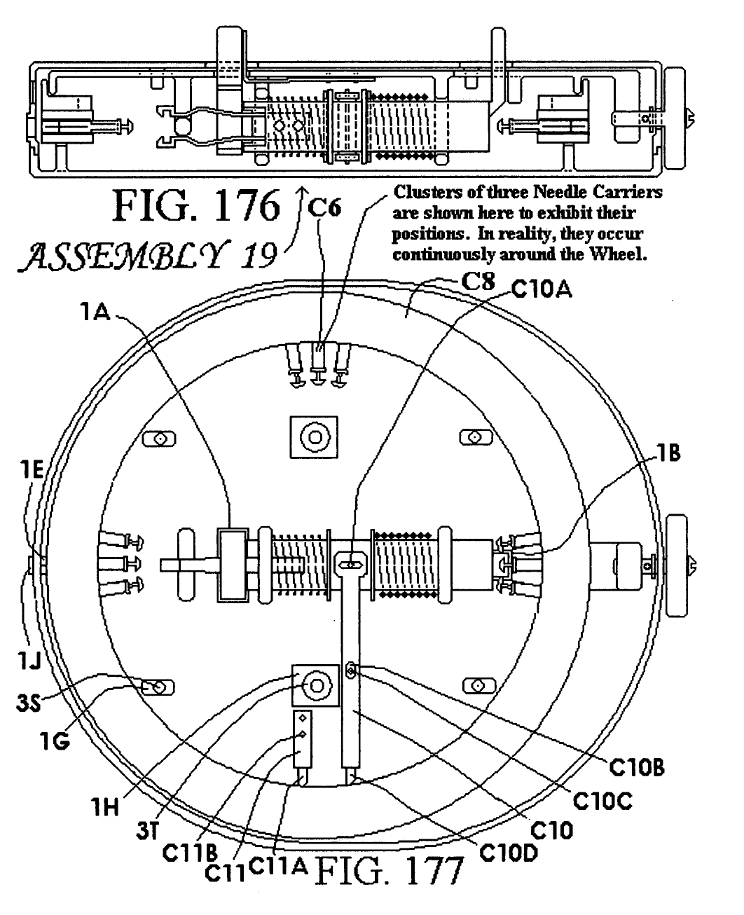

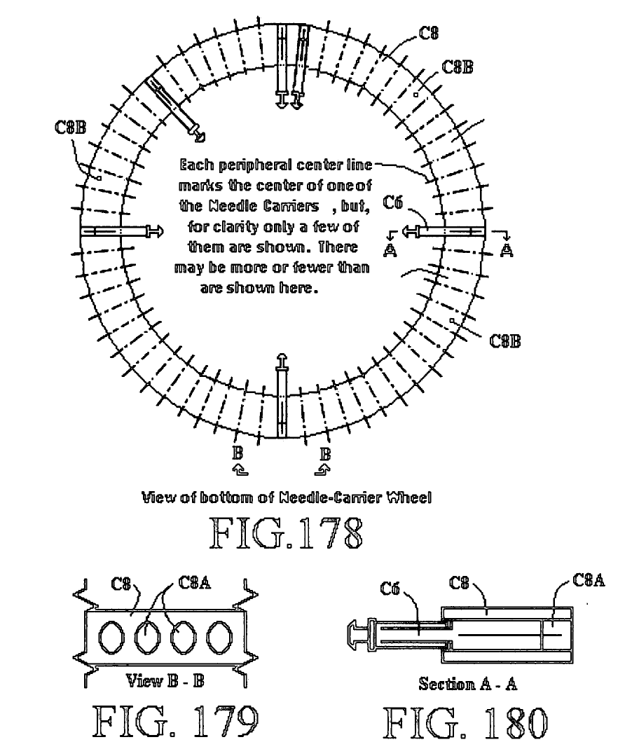

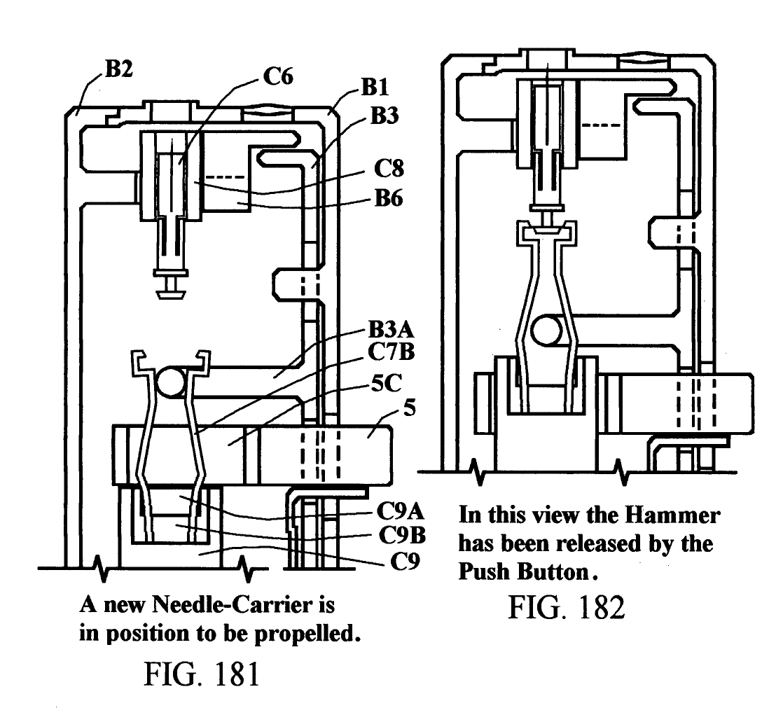

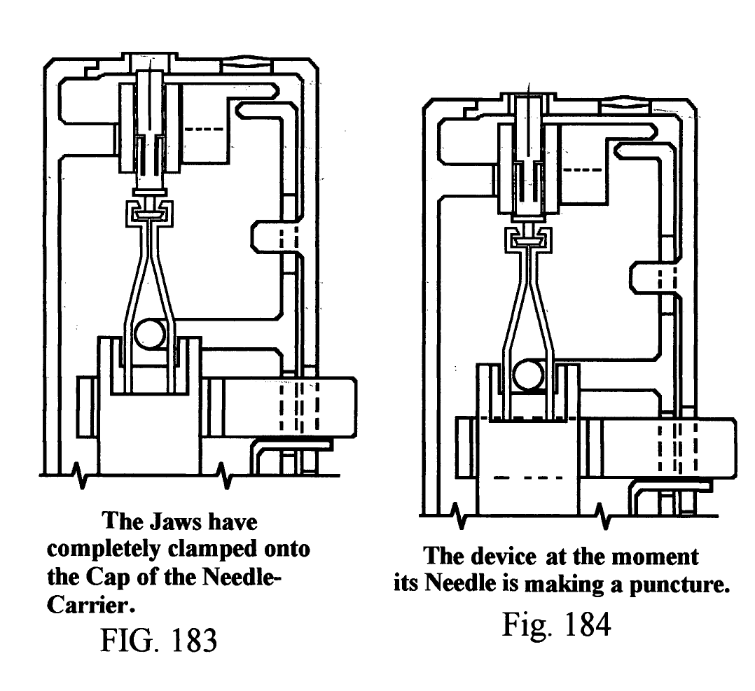

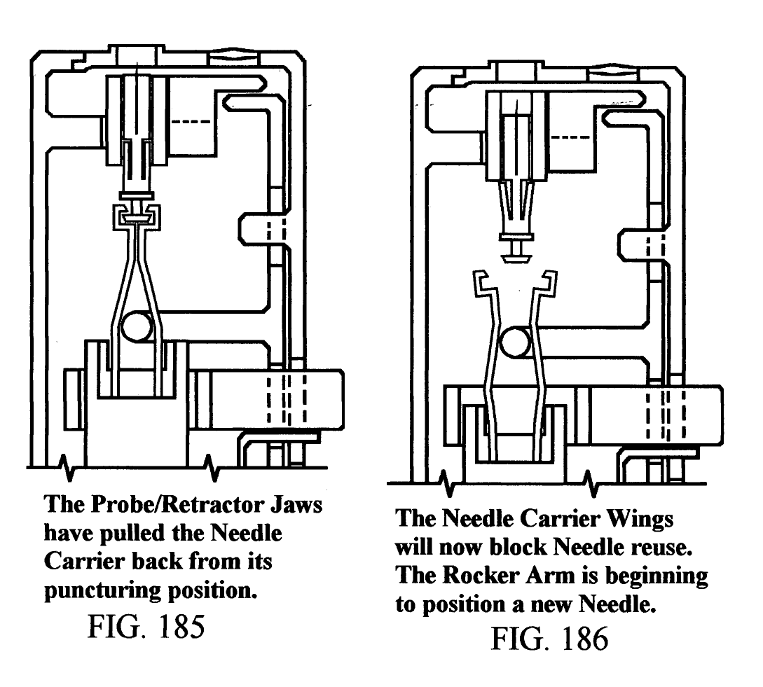

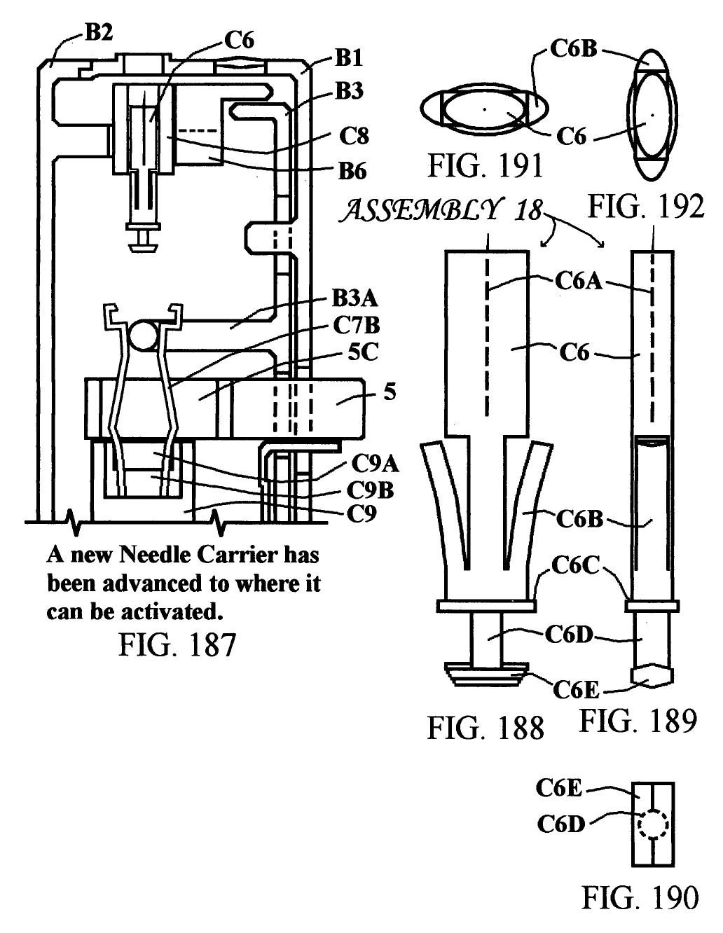

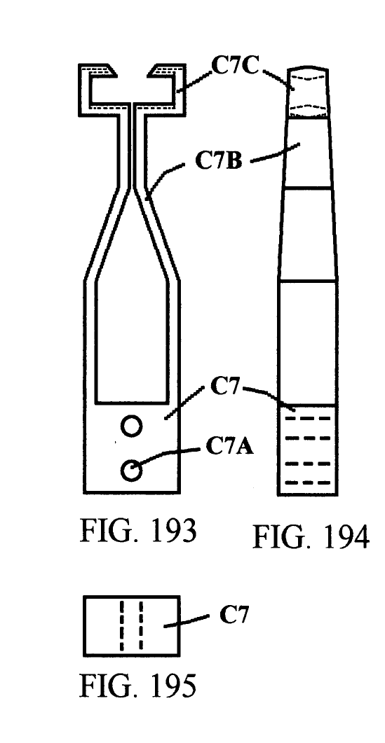

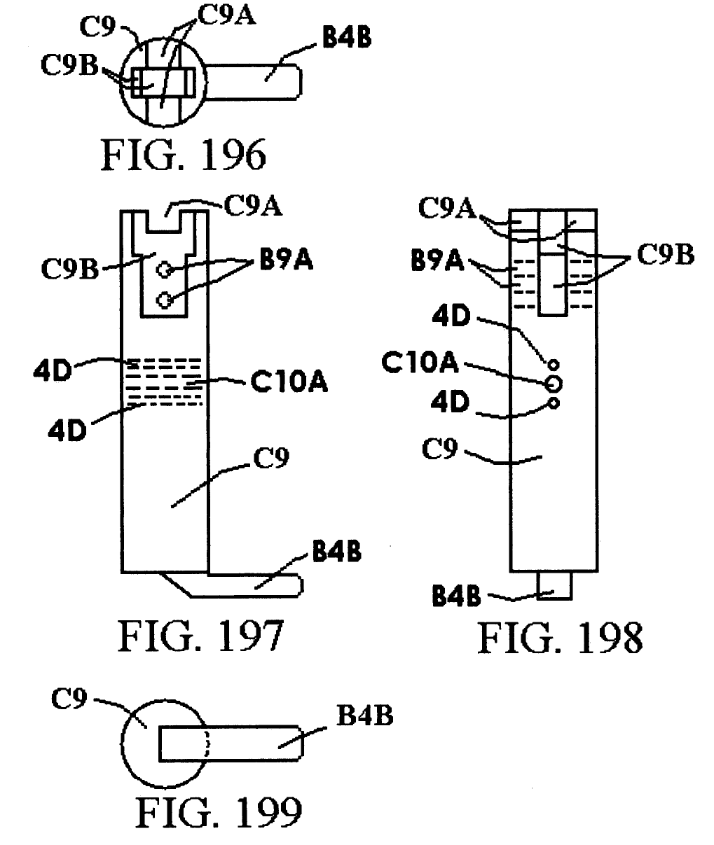

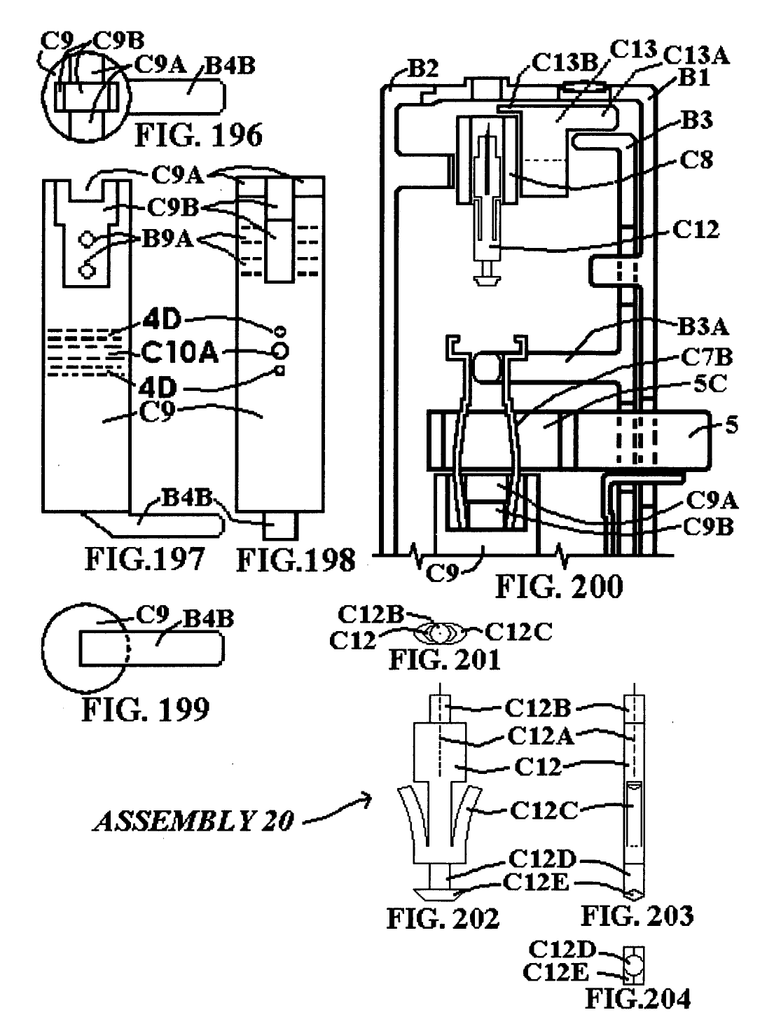

### DETAILED DESCRIPTION OF THE INVENTION

Referring to FIGS. 1-204 on the drawings detailing the RENEWABLE ROTARY SKIN LANCET, there are three different but quite similar devices shown, also an array of several different Needle Carrier and Probe/Retractor combinations are shown. It may be that more than one of these different devices will become preferred embodiments, or that a combination of the parts shown for more than one of the devices will be combined to make preferred embodiments of the present invention, for the preferred embodiment cannot be known until prototypes of the present invention are constructed and tested. Additionally, the preferred embodiment may be chosen depending on the manufacturing capabilities of the company selected to manufacture the device for marketing.

Nevertheless, each of the three different devices has the following characteristics in common with each other, making essentially the same device.

### 1. A case comprised of a lid and a bottom fastened together securely, and containing the mechanisms to puncture on demand a person's skin in order to obtain a small amount of blood for testing purposes.

### 2. An aperture in said case through which a needle may be projected into a person's skin.

### 3. A viewing window in said case, said viewing window to be equipped with an integral magnifying lens to more clearly make visible the numbers inside said case.

### 4. Numbers on the mechanism inside said case for indicating through said viewing window the number of the needle then positioned in said mechanism for being propelled into a person's skin.

### 5. A pushbutton projecting through said case to act as a trigger for initiating the propulsion or firing of said needle into a person's skin.

### 6. A cocking lever projecting through said case to cock said mechanism after each propulsion or firing of said needle.

### 7. A renewable, replaceable needle wheel in the mechanism, said wheel to contain a plurality of radially-positioned needle carriers.

8. A plurality of said needle carriers, each movable outwardly with restrictions to contain them in their slots and to keep them from, being propelled too far, and each bearing a needle made for the purpose of puncturing a person's skin. The shape of said needle carriers is to be complementary to the shape of the Probe/Retractors which are used to strike, propel, and retract said needle carriers.

### 9. A cylindrical hammer moving longitudinally within guides towards and then away from said needle aperture.

### 10. A main spring used to propel said hammer towards said needle aperture.

### 11. A rebound spring used to partially retract said hammer and needle carriers after they have been propelled towards a person's skin.

### 12. A sliding wheel carrier to which the mechanisms inside said case may be fastened and together moved closer to or further from said needle aperture.

13. A plurality of small bumps or rounded protrusions on the bottom of said wheel carrier for the purpose of reducing the friction between the bottom of said wheel carrier and the inner face of said case bottom when said wheel carrier is being moved for the purpose of adjusting the depth of penetration of said needles.

### 14. Protrusions from said case bottom which snugly, but not tightly, pass through said wheel carrier and keep said wheel carrier aligned laterally so it can slide only directly towards or directly from said needle aperture.

15. A turnable knob projecting from said case, the knob being used to move said sliding wheel carrier in gradual increments of distance towards and away from said needle aperture, thus adjusting the depth of penetration when a person's skin is punctured.

### 16. Protrusions on said wheel carrier for the purpose of limiting the distance said hammer can travel both towards and away from said needle aperture.

### 17. A mechanism within said case for advancing, after each firing of said hammer, the said wheel carrier in a rotational manner the distance required to move an unused needle carrier into a proper position for it to be propelled into a person's skin.

### 18. A mechanism built onto each of the said wheel carriers to prevent said needle wheel from rotating in a direction opposite the intended rotational direction.

19. Areas or protrusions on the inner, bottom side of said case lid to be used to meet the upper side of said needle wheel in order to closely govern unwanted vertical movement of said needle wheel, this being done to assure proper centering of a said needles in said needle aperture
20. A probe/retractor attached to said hammer, and moving in conjunction with said hammer, used to strike said needle carriers as a means of propelling their needles into a person's skin, and, after that is accomplished, used to retract said needle to a place where it will not be exposed to accidental skin puncturing.

### 21. A means of positively stopping any further rotation of said needle wheel after its last unused needle carrier has been used.

### 22. Slots, tunnels or grooves in said needle wheel into which said needle carriers can be inserted so that said needle carriers may freely slide within said needle wheel without adverse lateral movement.

### 23. The necessary provisions within said mechanisms whereby said needle wheel can be easily and correctly positioned when a new needle wheel is inserted into the mechanisms.

The three different devices are shown and referred to in the present invention as Alternate Device Number 1, Alternate Device Number 2, and Alternate Device Number 3. The extent and nature of said differences can be visualized on the drawings which accompany the present patent application.

The Needle-Carrier Wheel differs in each of the said devices. The Needle Carrier Wheel for Alternate Device Number 1 contains the Advancer Pits which are used in rotating said Wheel, while Alternate Device Number 2 and Alternate Device Number 3 have Needle Carrier Wheels which fit atop separate wheels known as Cog Wheels. The Cogs in the Cog Wheels are used in rotating those Needle Carrier Wheels. These differences are necessitated by the different means of advancing the Needle Carrier Wheel on its rotational path.

Each of the different Needle Carrier Wheels is equipped with a different type of slot, channel, or groove for containing said Needle Carriers. These differences are necessitated by the different shapes of the various Needle Carriers.

Several different combinations of Needle Carriers and Probe/Retractors are shown on the said drawing. These are able to be used, perhaps with moderate revisions, on the various Alternate Devices.

Alternate Device Number One uses a sort of flexible, cam-equipped finger, reaching into an Advancer Pit to rotate the Needle-Carrier Wheel when the Hammer of that device is fired.

Alternate Devices Number 2 and 3 use a Rocker Arm. Said Rocker Arm is necessitated because the change in design of said Needle-Carrier Wheels is necessitated by the use of different types of Needle Carriers. The Dog end of the Rocker Arm slips into a Cog in said Cog Wheel to provide rotation, while said Rocker Arm's opposite end is attached to said hammer. When said Hammer is cocked, said Dog end rotates said Cog Wheel, and, thereby, said Needle-Carrier Wheel. Upon the firing of said Hammer, cocking of said Hammer must take place before the device can be used again, and the Dog end then moves a new Needle Carrier into a firing position. Said Rocker Arm is attached to said Hammer via slotted holes. The slotted holes allow said Hammer, during the first part of its travel while being cocked, to retract the used Needle Carrier without starting to rotate the Needle-Carrier Wheel. During the last part of said Hammer's travel upon being cocked, the pin at the slotted holes alongside the Hammer will finally engage the end of the slotted holes and only then will the rotation of the Needle-Carrier Wheel be started. Said slotted holes are the key element in this dual function of said Rocker Arm.

See “IDENTIFICATION AND DESCRIPTION OF THE FIGURES ON THE DRAWINGS FOR THE RENEWABLE ROTARY SKIN LANCET” for some descriptive language related to certain drawings and figures. Also see “BRIEF SUMMARY OF THE INVENTION” for additional descriptive material.

### RENEWABLE ROTARY SKIN LANCET PARTS AND FEATURES

### ** Used Unchanged in Alternate Device No. 1, Alternate Device No. 2, and Alternate Device No. 3

1. CASE BOTTOM ONE
1A.** Pushbutton Opening
1B.** Cocking-Lever Opening
1C.** Adjusting-Stud Opening
1D.** Needle Aperture
1E.** Centering Notch
1F.** Perimeter Nesting-Edge
1G.** Carrier Alignment Posts
1H.** Lid-Attachment Screw Posts
1J.** Raised Rim at Needle Aperture
2. CASE LID ONE
2A.** Perimeter Nesting-Edge
2B.** Attachment Screws
2C.** Attachment-Screw Openings
2D.** Lancet Number Viewing Opening
3. WHEEL CARRIER ONE
3A.** Adjusting Stud
3B.** Adjusting-Stud Washer
3C.** Adjusting-Stud Washer pins
3D.** Adjusting Knob
3E.** Adjusting-Knob Screw
3F.** Glide Bumps
3G. Hammer Stop
3H.** Upper Hammer Guide
3J.** Pushbutton-Spring Opening
3K.** Lower Hammer Guide
3L.** Hammer-Guide Openings
3M. Cocking-Lever Stop
3N. Cocking-Lever Stop Screw
3O. Advancer Adjustment Screw
3P.** Adjusting Arm
3Q.** Adjusting-Arm Threaded Hole
3R.** Rubbing Rail
3S.** Alignment-Post Openings
3T.** Lid-Attachment Post Openings
3U.** Pushbutton Opening
3V.** Cocking-Lever Opening
3W.** Wheel Stop Tab
3X. Detent Ramp
3Y. Detent leaf with protrusion
3Z. Detent-Leaf Screws
4. HAMMER ONE
4A.** Spring
4B.** Rebound Spring
4C.** Spring Washers
4D.** Spring-Washer Pins
4E. Probe/Retractor One
4F. Retractor Slot on Probe/Retractor
4G. Cocking Lever
4H. Advancer Clamping Slot
4J. Advancer Clamping Screw and Nut
4K. Advancer
4K. Slot for Probe/Retractor
5.** PUSHBUTTON
5A.** Pushbutton Spring
5B.** Pushbutton-Spring Attachment Screws And Holes
5C.** Hammer Opening
6. NEEDLE CARRIER WHEEL ONE
6A. Wheel Rubbing Rail
6B. Needle-Carrier Slots
6C. Advancer Pits
6D. Stop Post
6E. Detent Notches
6F. Null Position Between Probe/Retractor Slots.
6G. Probe/Retractor Slot for Wheel Insertion
6H. Probe/Retractor Slot for Wheel Removal
A18. Shoulder Pocket in Wheel if Needle Carrier A11 is used with this Wheel.
7. NEEDLE CARRIER ONE
7A. Needles
7B. Needle Carrier Wings
7C. Stop Protrusions
7D. Retraction Protrusions
8. ** NUMBERING, LETTERING, AND DIRECTIONAL ARROWS
9. NEEDLE CARRIER TWO
9A. Needle Carrier Wings
9B. Stop Shoulder
9C. Clamping Jaws
9D. Needles
10. PROBE/RETRACTOR TWO
10A. Retractor Knobs
A1. NEEDLE CARRIER THREE
A5. Flexible Shoulder Arm
A6. Stop Shoulder
A7. Clamping-Jaw Arm
A8. Clamping Jaw
A9. Clamping-Jaw Beveled End
A10. Clamping-Jaw Shoulder
A2. PROBE/RETRACTOR THREE
A3. Retraction Cap
A4. Beveled End on Retraction Cap
A11. NEEDLE CARRIER FOUR
A12. Flexible Shoulder Arm
A13. Stop Shoulder
A14. Clamping-Jaw Arm
A15. Clamping Jaw
A16. Clamping-Jaw Beveled End
A17. Clamping-Jaw Shoulder
B1. CASE BOTTOM TWO
B3. Lancet Number Viewing Opening
B2. CASE LID TWO
B2E. Pressure Post
B5. WHEEL CARRIER TWO
B3A. Hammer Stop/Retractor Spreader
C10E. Rocker Arm Pivot Pedestal and Pivot Pin
C11C. Detent Dog Pedestal and Fastenings
B6. COG WHEEL
B6A. Cog Wheel Cogs
B6B. Cog Wheel Rubbing Rail
B6C. Cog Wheel Stop Post
B6D. Cog Faces
C11D. Pins For Positioning Needle Carrier Wheel
B8. NEEDLE CARRIER WHEEL TWO
B8A. Positioning-Pin Socket Holes
B7E. Cover Lips
B7F. Needle Carrier Blocking Flaps
B7G. Slot For Needle Carrier
B7H. Gap between Cover Lips and Blocking Flaps
B7. NEEDLE CARRIER FIVE
B7A. Needle
B7B. Needle Carrier Cap
B7C. Needle Carrier Stop Rim
B7D. Needle Carrier Cap Connector
B4. HAMMER TWO
B4A. Probe/Retractor Slot
B4B. Cocking Lever
B9A. Probe/Retractor Pins and Holes
C10. ROCKER ARM
C10A. Rocker Arm Pin and Hole
C10B. Elongated Hole for Pivot Pin
C10C. Rocker Arm Pivot Pin
C10D. Rocker Arm Dog, Spring, and Retaining Pin
C10E. Elongated Holes in Rocker Arm at the Hammer End
C11. COG WHEEL DETENT BODY
C11A. Detent Dog, Spring, and Retaining Pin
C11B. Detent Body Fastenings
B9. PROBE/RETRACTOR FOUR
B9A. Holes for Fastenings
B9B. Flexible Arm on Probe/Retractor
B9C. Probe/Retractor Jaws
C6. NEEDLE CARRIER SIX
C6A. Needle
C6B. Stop Arms
C6C. Stop Rim
C6D. Cap Connector
C6E. Cap
C7. PROBE/RETRACTOR FIVE
C7A. Holes for Fastenings
C7B. Flexible Arms
C7C. Jaws
C8. NEEDLE CARRIER WHEEL THREE
C8A. Slots for Needle Carriers
C8B. Positioning-Pin Socket Holes
C9. HAMMER THREE
C9A. Recess for Spreader
C9B. Recess for Probe/Retractor socketing and flexing.
C12. RIMLESS NEEDLE CARRIER SIX
C12A. Embedded needle
C12B. Bypassing projection on needle carrier C12
C12C. Stop arms
ASSEMBLY 1. The entire Alternate Device Number 1.
C12D. Cap Connector
C12E. Cap
C13. COG WHEEL WITH CONTINUOUS BUMPER FOR NEEDLE STOP
C13A. Cog Wheel rubbing rail
C13B. Continuous bumper rim on Cog Wheel
ASSEMBLY 2. Case Bottom 1, Case Lid 2.
ASSEMBLY 3. Case Lid 2 and Magnifying Lancet Viewing Window 2D.
ASSEMBLY 4. Wheel Carrier 3, Adjusting Screw 3N, & Adjusting Screw 30.
ASSEMBLY 5. Hammer 4, Advancer 4K, and Clamping Screw/Nut 4J.
ASSEMBLY 6. Pushbutton 5 and Spring 5A.
ASSEMBLY 7. The entire Alternate Device Number 2.
ASSEMBLY 8. Case Lid B2 and Case Bottom B1.
ASSEMBLY 9. Case Bottom B1 and Magnifying Lancet Viewing Window B3.
ASSEMBLY 10. Cog Wheel B6 and positioning pins C11D.
ASSEMBLY 11. Cog Wheel Detent C11 and its dog, spring, and retaining pin.
ASSEMBLY 12. Rocker Arm C10, its Dog, Spring, & Retaining & Pivot Pins.
ASSEMBLY 13. Needle Carrier 7 and Needle 7A.
ASSEMBLY 14. Needle Carrier 9 and Needle 9D.
ASSEMBLY 15. Needle Carrier A1 and Needle 7A.
ASSEMBLY 16. Needle Carrier A11 and Needle 7A.
ASSEMBLY 17. Needle Carrier B7 and Needle B7A.
ASSEMBLY 18. Needle Carrier C6 and Needle C6A.
ASSEMBLY 19. Entire Alternate Device Number Three.
ASSEMBLY 20. Needle Carrier C12 and Needle C12A.

What is claimed is:

1. A cased renewable rotary lancet device having a plurality of numbered, sterile, non-reusable, untouchable needles mounted radially on a renewable wheel which can be rotated by a dual-purpose cog wheel mechanism such that a new needle is moved into a position wherein a hammer mechanism, after being manually cocked, and upon being released and fired by a pushbutton, will propel the needle an adjustable depth into a person's skin to make skin punctures for the purpose of obtaining blood to be used in making analytical tests on that blood, after which, when each of the needles have been used one time, the wheel cannot be rotated further, the needles cannot be used again, not even accidentally, the needles will have been hidden from human touch, and the wheel can be removed and replaced with a new wheel bearing new needles, said renewable rotary skin lancet device comprising:
[a] a case one;
[b] a case two;
[c] a needle-carrier wheel one;
[d] a needle-carrier wheel two;
[e] a needle-carrier wheel three;
[f] a hammer one, including its attached advancer and cocking lever;
[g] a hammer two, including its cocking lever;
[h] a hammer three, including its cocking lever;
[k] a wheel carrier one;
[l] a wheel carrier two;
[m] a rocker arm;
[n] a pushbutton;
[o] a needle carrier one, with exposesd needle point;
[p] a needle carrier two, with exposesd needle point;
[q] a needle carrier three, with exposesd needle point;
[r] a needle carrier four, with exposesd needle point;
[s] a needle carrier five, with exposesd needle point;
[t] a needle carrier six, two varieties, one lacking a stop rim, both with an exposesd needle point;
[u] a probe/retractor one;
[v] a probe/retractor two;
[w] a probe/retractor three;
[x] a probe/retractor four;
[y] a probe/retractor five
[z] a cog wheel, two types, one with a raised needle carrier bumper rim;
[aa] a detent leaf with protrusion;
[bb] a cog wheel detent;
[cc] miscellaneous numbering, lettering, and markings; and
[dd] miscellaneous fastenings, pins, springs, washers, screws, bolts, and nuts.

2. A reusable lancet device as recited in claim 1 wherein said cases will have a lid part and a bottom part comprising:
[a] openings for those parts, including at least the needle aperture, recited in claim 1 requiring apertures in said cases for the proper operation of said parts;
[b] a lancet-number viewing window wherein a magnifying lens is fastened;
[c] areas or projections supplying pressure to hold the parts recited in claim 1 inside said cases in a workable position;
[d] projections inside said cases for the operational alignment of parts recited in claim 1;
[e] projections inside said cases for fastenings recited in claim 1;
[f] instructional lettering and markings as recited in claim 1;
[g] fastenings as recited in claim 1 used to keep the lid part and bottom part of the cases joined;
[h] said cases as recited in claim 1 in which said cases are made of metal;
[k] said cases as recited in claim 1 in which said cases are made of plastic; and
[l] said cases as recited in claim 1 in which said cases are made of a combination of plastic and metal.

3. A reusable lancet device as recited in claim 1 wherein said needle-carrier wheel one is comprised of:
[a] a flat top surface, an irregular bottom surface, a circular outer edge, and a circular inner edge;
[b] tunnels between said inner edge and said outer edge, evenly spaced, parallel to imaginary radial lines from the center of said needle-carrier wheel one, spaced continuously around said needle-carrier wheel one as closely as possible without disturbing any of the functions of said reusable lancet device, wherein at least some of the needle carriers recited in claim 1 will fit in said tunnels with clearances which will allow said needle carriers to freely slide longitudinally in said tunnels, but also with clearances which will not allow lateral movement of said needle carriers to any extent which will be visible to normal, visually-unassisted eyes;
[c] grooves at the bottom surface, inner edge, of said needle-carrier wheel one to allow insertion and removal of said needle-carrier wheel one when the hammer recited in claim 1 has been fired and its probe/retractor extends into the space normally occupied by needle-carrier wheel one;
[d] detent notches, one at each needle carrier recited in claim 1, in the bottom surface, inner edge, of said needle-carrier wheel one wherein the protrusion on the detent leaf recited in claim 1 can project into said detent notches to stabilize said needle-carrier wheel one and reduce the chance of the said needle carriers being misaligned when they are to be centered with the needle aperture recited in claim 2;
[e] a rubbing rail at the bottom of the outer edge of said needle-carrier wheel one wherein said rubbing rail fits outside a complementary rubbing rail located at the outside edge of the wheel carrier one recited in claim 1 such that said needle-carrier wheel one maintains a constant common center with said wheel carrier one so long as said needle-carrier wheel one is positioned for rotation;
[f] advancer pits, as recited in claim 1, one near each needle carrier, on the inner edge of said needle-carrier wheel one at its upper surface, each to serve as a cog wherein said pits can receive the distal end of the advancer recited with hammer one in claim 1, such that movement of said advancer will, in its path of movement as the hammer recited in claim 1 is cocked, advance said needle-carrier wheel one the distance required to position the next unused needle carrier where the probe/retractor one recited in claim 1 can strike said unused needle carrier and propel it to the position whereby the needle point in said needle carrier can puncture skin if skin is then present at the needle aperture recited in claim 2.
[g] the ability of said needle-carrier wheel one to be rotated while said needle carriers are being used one by one until all of said needle carriers have been used one time;
[h] an integrally-formed, cylindrical post located on the bottom of said needle-carrier wheel one to serve as a stop post wherein said stop post will, at the moment the final unused needle carrier is in a usable position at said needle aperture, meet a wheel stop tab located at a coordinated position on the wheel carrier one recited in claim 1;
[k] a number, as recited in claim 1, on the surface of said needle-carrier wheel one adjacent to each needle carrier, with said numbers being sequential, consecutive, and indelibly marked in a dark color on said needle-carrier wheel one;
[l] directions and markings, as recited in claim 1, marked on said needle-carrier wheel one indelibly in a dark color;
[m] said needle-carrier wheel one as recited in claim 1 in which said needle-carrier wheel one is made of metal;
[n] said needle-carrier wheel one as recited in claim 1 in which said needle-carrier wheel one is made of plastic; and
[o] said needle-carrier wheel one as recited in claim 1 in which said needle-carrier wheel one is made of a combination of metal and plastic.

4. A reusable lancet device as recited in claim 1 wherein said needle-carrier wheel two is comprised of:
[a] a flat top surface, an irregular bottom surface, a circular outer edge, and a circular inner edge;
[b] channels between said inner edge and said outer edge, evenly spaced, parallel to imaginary radial lines from the center of said wheel, spaced continuously around said wheel as closely as possible without disturbing any of the functions of said reusable lancet device, wherein at least some of the said needle carriers recited in claim 1 will fit in said channels with clearances which will allow said needle carrier to freely slide longitudinally in said channels, but with clearances which will not allow lateral movement of said needle carriers to any extent which will be visible to normal, visually-unassisted eyes;
[c] a plurality of positioning-pin socket holes matching the locations of positioning pins located in the separate cog wheel, as recited in claim 1, upon which said needle-carrier wheel two is to be inserted;
[d] cover lips arching part way over said needle carriers part way along each side of said channels recited in claim 4, said cover lips to be flexible enough such that the needle carriers can be inserted into said channels by pressing the needle carrier between said cover lips into said channels;
[e] blocking flaps arching over and part way along each side of said needle-carriers channel recited in claim 4[b], more specifically, along that part of said channel near the outer edge of needle-carrier wheel two, said blocking flaps to have been formed when they were made so that absent a needle carrier in the blocking-flaps part of the channel, said blocking flaps will return to their natural state, and will thereby block said channels so a partially-retracted, used needle carrier cannot again be fully propelled in the channel to puncture skin;
[f] a gap between said blocking flaps and said cover lips such that said blocking flaps do not interfere with said cover lips;
[g] instructional signage marked on the top surface of said needle-carrier wheel two, using an indelible dark color; and
[h] a material forming said needle-carrier wheel two as recited in claim 1 in which said needle-carrier wheel two is made of a plastic having the flexible qualities required for the proper composition of said cover lips and said blocking flaps.

5. A reusable lancet device as recited in claim 1 wherein said needle-carrier wheel three is comprised of:
[a] a flat top surface, a flat bottom surface, a circular outer edge, and a circular inner edge;
[b] tunnels between said inner edge and said outer edge, evenly spaced, parallel to imaginary radial lines from the center of said needle-carrier wheel three, spaced continuously around said needle-carrier wheel three as closely as possible without disturbing any of the functions of said reusable lancet device, wherein some of said needle carriers recited in claim 1 will fit in said tunnels with clearances which will allow said needle carrier to freely slide longitudinally in said tunnels, but with clearances which will not allow lateral movement of said needle carriers to any extent which will be visible to normal, visually-unassisted eyes;
[c] a plurality of positioning-pin socket holes matching the locations of positioning pins located in the separate cog wheel as recited in claim 1 upon which said needle-carrier wheel three is to be inserted;
[d] instructional signage marked as needed on the top surface of said needle-carrier wheel three, using an indelible dark color;
[e] said wheel as recited in claim 1 in which said needle-carrier wheel three is made of metal;
[f] said wheel as recited in claim 1 in which said wheel is made of plastic; and
[g] said wheel as recited in claim 1 in which said wheel is made of a combination of metal and plastic.

6. A reusable lancet device as recited in claim 1 wherein hammer one and its advancer are comprised of:
[a] a hammer one comprising an attached, removable advancer, also a socket wherein a probe/retractor can be fastened, and wherein a cocking-lever arm used to cock said hammer is integrally formed and a part of said hammer one;
[b] a cocking lever as recited in claim 1 made integrally with said hammer one, and projecting through the case recited in claim one where it can be grasped by a user's finger and pulled to cock said hammer one preparatory to its being fired;
[c] a cylindrical compression spring as recited in claim 1, encompassing said hammer one such that said hammer one is propelled by said spring towards the needle aperture as recited in claim 2 when said hammer one is released by the pushbutton recited in claim 1;
[d] a cylindrical, compression, rebound spring as recited in claim 1, encompassing said hammer one such that when said hammer one has been propelled towards said needle aperture, and the needle carrier recited in claim 1 has been conjunctively moved to where its needle can make a skin puncture, said rebound spring will cause said hammer one to partially withdraw, along with the probe/retractor as recited in claim 1, and along with the needle carrier as recited in claim 1;
[e] an advancer fastened onto said hammer one such that when said hammer one is being cocked said advancer will project into an advancer pit as recited in claim three, and will advance said needle-carrier wheel one the distance required to position the next unused needle carrier where the probe/retractor one recited in claim 1 can strike said unused needle carrier and propel it to the position whereby the needle point in said needle carrier can puncture skin if skin is then present at the needle aperture recited in claim 1.
[f] a hammer one weighing an amount of weight such that its momentum after being propelled by said spring recited in claim 6 [c] will be enough to carry said hammer one by momentum such that its action can result in an effective skin puncture by said needle contained in said needle carrier;
[g] a hammer one weighing an amount of weight such that its momentum, while being retracted by said rebound spring recited in claim 6 [d], will be sufficient to withdraw the then-connected needle carrier such that the needle point contained in said needle carrier is hidden from human touch within the confines of said needle-carrier wheel one;
[h] said hammer one as recited in claim 1 in which said hammer one is made of metal;
[k] said hammer one as recited in claim 1 in which said hammer one is made of plastic; and
[l] said hammer one as recited in claim 1 in which said hammer one is made of plastic in combination with metal.

7. A reusable lancet device as recited in claim 1 wherein hammer two is comprised of:
[a] a hammer two comprising a socket wherein a probe/retractor can be fastened, and wherein a cocking-lever arm used to cock said hammer is an integrally-formed part of said hammer two;
[b] a cocking lever as recited in claim 1 made integrally with said hammer two, and projecting through the case recited in claim one where it can be grasped by a user's finger and pulled to cock said hammer two preparatory to its being fired;
[c] a cylindrical compression spring as recited in claim 1, encompassing said hammer two such that said hammer two is propelled towards the needle aperture as recited in claim 2 when said hammer two is released by the pushbutton recited in claim 1;
[d] a cylindrical, compression, rebound spring as recited in claim 1, encompassing said hammer two such that when said hammer two has been propelled towards said needle aperture, and the needle carrier recited in claim 1 has been conjunctively moved to where its needle can make a skin puncture, said rebound spring will partially withdraw said hammer one, along with the probe/retractor as recited in claim 1, and along with the needle carrier as recited in claim 1;
[f] a hammer two weighing the amount of weight such that its momentum after being propelled by said spring recited in claim 7 [c] will be enough to carry said hammer two such that its action can result in an effective skin puncture by said needle contained in said needle carrier;
[g] a hammer two weighing the amount of weight such that its momentum, while being retracted by said rebound spring recited in claim 7 [d], will be sufficient to withdraw the then-connected needle carrier such that the needle point contained in said needle carrier is hidden from human touch within the confines of said needle-carrier wheel two;
[h] a hole for connecting the rocker arm recited in claim 1 to said hammer two with a pin, said rocker arm having a slotted hole for effecting said connection such that said rocker arm will have a delayed action in its rotation of needle carrier wheel two when hammer two is cocked;
[k] said hammer two as recited in claim 1 in which said hammer two is made of metal;
[l] said hammer two as recited in claim 1 in which said hammer two is made of plastic; and
[m] said hammer two as recited in claim 1 in which said hammer two is made of plastic in combination with metal.

8. A reusable lancet device as recited in claim 1 wherein hammer three is comprised of:
[a] a hammer three comprising sockets and recesses wherein a probe/retractor can be fastened and such that parts of said reusable lancet device can remain undisturbed by said hammer as said hammer three is cocked or fired, and wherein a cocking-lever arm used to cock said hammer is an integrally-formed part of said hammer three;
[b] a cocking lever as recited in claim 1 made integrally with said hammer three, and projecting through the case recited in claim one where it can be grasped by a user's finger and pulled to cock said hammer three preparatory to its being fired;
[c] a cylindrical compression spring as recited in claim 1, encompassing said hammer three such that said hammer three is propelled towards the needle aperture as recited in claim 2 when said hammer three is released by the pushbutton recited in claim 1;
[d] a cylindrical, compression, rebound spring as recited in claim 1, encompassing said hammer three such that when said hammer three has been propelled towards said needle aperture, and the needle carrier recited in claim 1 has been conjunctively moved to where its needle can make a skin puncture, said rebound spring will partially withdraw said hammer one, along with the probe/retractor as recited in claim 1, and along with the needle carrier as recited in claim 1;
[f] a hammer three weighing the amount of weight such that its momentum after being propelled by said spring recited in claim 7 [c] will be enough to carry said hammer three such that its action can result in an effective skin puncture by said needle contained in said needle carrier;
[g] a hammer three weighing the amount of weight such that its momentum, while being retracted by said rebound spring recited in claim 7 [d], will be sufficient to withdraw the then-connected needle carrier such that the needle point contained in said needle carrier is hidden from human touch within the confines of said needle-carrier wheel three as recited in claim 1;
[h] a hole for connecting the rocker arm recited in claim 1 to said hammer three with a pin, said rocker arm having a slotted hole for effecting said connection such that said rocker arm will have a delayed action in its rotation of needle-carrier wheel three as recited in claim 1 when hammer three is cocked preparatory to its being fired;
[k] said hammer three as recited in claim 1 in which said hammer three is made of metal;
[l] said hammer three as recited in claim 1 in which said hammer three is made of plastic; and
[m] said hammer three as recited in claim 1 in which said hammer three is made of plastic in combination with metal.

9. A reusable lancet device as recited in claim 1 wherein wheel carrier one is comprised of:
[a] a circular floor to which other of its parts are integrally formed;
[b] an adjusting arm integral with said circular floor of wheel carrier one, and serving as a member whereby a threaded adjusting stud can be attached such that rotation of said threaded adjusting stud will move said wheel carrier one longitudinally with respect to said adjusting stud;
[c] a threaded adjusting stud as recited in claim 9[b] coupled to an adjusting knob, said adjusting knob equipped with pins, washers, and screws, whereby the rotation of said knob will move said wheel carrier one longitudinally with respect to said adjusting stud, said adjusting knob, and the needle aperture recited in claim 2 and thereby will provide a means for adjusting said wheel carrier one, needle-carriers one, two, three, or four, as recited in claim 1, and with one of said needle carriers positioned where its needle point will be propelled when said hammer one and probe/retractors one, two, or three, as recited in claim 1, are fired, whereby said needle point will, as its placement is adjusted, have its penetration into skin presented at said needle aperture reduced or increased as the need arises;
[d] a circular rubbing rail rising from the perimeter of said floor of wheel carrier one, and so disposed that needle-carrier wheel one's rubbing rail will encircle wheel carrier one's rubbing rail whereby said wheel carrier one's rubbing rail will guide needle-carrier wheel one as said needle-carrier wheel one rotates with a common center existing between said wheel carrier one and needle-carrier wheel one;
[e] a plurality of glide bumps on the surface of wheel carrier one which abuts the inner surface of the case bottom part recited in claim 2, whereby any friction between said wheel carrier one and said case at the time the position of wheel carrier one is being adjusted will be reduced;
[f] a wheel stop tab, an integral part of wheel carrier one, positioned so it will stop the rotation of needle-carrier wheel one when said wheel stop tab meets the stop post recited in claim 3;
[g] guides for the hammer one recited in claim 1, said guides constructed as integral parts of said wheel carrier one;
[h] stops limiting the longitudinal movements of hammer one when said hammer one is being cocked or fired, with said stops constructed as integral parts of wheel carrier one;
[k] a detent ramp upon which the detent leaf with protrusion recited in claim one can be fastened, with said detent ramp constructed as an integral part of wheel carrier one;
[l] openings as required in wheel carrier one for the penetration or attachment of other parts of said reusable lancet device;
[m] parts required for fastenings;
[n] said wheel carrier one as recited in claim 1 in which said wheel carrier one is made of metal;
[o] said wheel carrier one as recited in claim 1 in which said wheel carrier one is made of plastic; and
[p] said wheel carrier one as recited in claim 1 in which said wheel carrier one is made of a combination of plastic and metal.

10. A reusable lancet device as recited in claim 1 wherein wheel carrier two is comprised of:
[a] a circular floor to which other of its parts are integrally formed;
[b] an adjusting arm integral with said circular floor of wheel carrier two, and serving as a member whereby a threaded adjusting stud can be attached such that rotation of said threaded adjusting stud will move said wheel carrier two longitudinally with respect to said adjusting stud;
[c] a threaded adjusting stud as recited in claim 10[b] coupled to an adjusting knob, said adjusting knob equipped with pins, washers, and screws, whereby the rotation of said knob will move said wheel carrier two longitudinally with respect to said adjusting stud, said adjusting knob, and the needle aperture recited in claim 2 and thereby will provide a means for adjusting said wheel carrier two, needle-carrier wheel two or three, and the needle carrier five or six positioned where the needle point of needle carrier five or six will be propelled when said hammer two or three and probe/retractor four or five are fired, whereby said needle point will, as its placement is adjusted, have its penetration into skin presented at said needle aperture reduced or increased as the need arises;
[d] a circular rubbing rail rising from the perimeter of said floor of wheel carrier two, and so disposed that the rubbing rail of the cog wheel recited in claim 1 will encircle wheel carrier two's rubbing rail so that said wheel carrier two's rubbing rail will guide said cog wheel as said cog wheel, coupled to said needle-carrier wheel two or three rotates with a common center existing between said wheel carrier two and needle-carrier wheel two or three;
[e] a plurality of glide bumps on the surface of wheel carrier two which abuts the inner surface of the case bottom part recited in claim 2, whereby any friction between said wheel carrier two and said case at the time the position of wheel carrier two is being adjusted will be reduced;
[f] a wheel stop tab positioned so it will stop the rotation of said cog wheel and its coupled needle-carrier wheel two or three when said wheel stop tab meets the stop post which is an integral part of said cog wheel;
[g] guides for the hammer two and three recited in claim 1, said guides constructed as integral parts of said wheel carrier two;
[h] stops limiting the longitudinal movements of hammer two or three, and for spreading the jaws of probe/retractor four or five when said hammer two or three is being cocked or fired, with said stops constructed as integral parts of wheel carrier two;
[k] a rocker arm pivot pedestal, rising from and integral with the inner floor of wheel carrier two, for supporting the rocker arm recited in claim 1;
[l] a detent-dog pedestal, rising from and integral with the inner floor of wheel carrier two, for supporting the cog wheel detent recited in claim 1 and referred to as a detent dog and as shown on the accompanying drawings;
[m] openings as required on wheel carrier two for the penetration or attachment of other parts of said reusable lancet device;
[n] parts required for fastenings;
[o] said wheel carrier two as recited in claim 1 in which said wheel carrier two is made of metal;
[p] said wheel carrier two as recited in claim 1 in which said wheel carrier two is made of plastic; and
[q] said wheel carrier two as recited in claim 1 in which said wheel carrier two is made of a combination of metal and plastic.

11. A reusable lancet device as recited in claim 1 wherein a rocker arm as recited in claim 1 is used to rotate the cog wheel recited in claim 1 with its coupled needle carrier wheel two or three as recited in claim 1, said rocker arm comprised of:
[a] slotted holes where said rocker arm attaches by a pin to hammer two or hammer three, said slotted holes made so said hammer two or three will first retract whichever probe/retractor is then involved, and only after said retraction will the rocker arm engage said cog wheel and move said cog wheel, with its accompanying needle-carrier wheel two or three, such that an unused needle carrier will be placed where it can be propelled when the involved hammer two or three is fired;
[b] slotted holes, located a distance along the length of said rocker arm, and of such hole length as is decided by trial and error, such that, because of their slotted nature, there will be no conflicting pressure exerted on the pin recited in claim 11[a];
[c] a bevel-ended, spring-loaded finger used to engage a cog face in said cog wheel for the purpose of rotating said cog wheel and its coupled needle-carrier wheel two or three;
[d] a retaining pin through said spring-loaded finger to retain said finger without impeding its required movements;
[e] parts required for fastenings, washers, and springs;
[f] said rocker arm as recited in claim 1 in which said rocker arm is made of metal;
[g] said rocker arm as recited in claim 1 in which said rocker arm is made of plastic; and
[h] said rocker arm as recited in claim 1 in which said rocker arm is made of a combination of metal and plastic.

12. A reusable lancet device as recited in claim 1 wherein a pushbutton as recited in claim 1 will be comprised of:
[a] a button part made to protrude through an opening in the case recited in claim 1, said part serving as a means of actuating a rectangular ring resting in the path of, and retaining and blocking, the travel of hammer one, two, or three, such that pressing on the protruding part of the pushbutton will release said hammer one, two, or three for firing;
[b] a ring made integrally with the button part recited in claim 12[a] to serve to block and retain said hammer one, two, or three until said button part is pressed, and then allowing said hammer one, two, or three to pass through said ring during the firing process;
[c] a spring used to provide resistance to a push on said button part, such that said ring will maintain its blocking and retaining function until a push on said button part can overcome said resistance so said hammer one, two, or three can be released;
[d] fastenings required for enabling said pushbutton to function;
[f] said pushbutton as recited in claim 1 in which said pushbutton is made of metal;
[g] said pushbutton as recited in claim 1 in which said pushbutton is made of plastic; and
[h] said pushbutton as recited in claim 1 in which said pushbutton is made of a combination of metal and plastic.

13. A reusable lancet device as recited in claim 1 wherein Needle carrier one as recited in claim 1 is comprised of:
[a] a needle made from a cylindrical rod of metal pointed on at least the distal end, and embedded in the body of said needle carrier one such that the needle will remain fixedly-embedded at least during the use of said needle carrier and needle for lancet purposes;
[b] flexible wings protruding from the body of said needle carrier one at the end opposite the end where said needle is projecting;
[c] protrusions on said wings such that complementary grooves on probe/retractor one can grasp said wings for the purpose of propelling and retracting said needle carrier one;
[d] protrusions on said wings such that needle carrier one must stop when said protrusions meet the inner edge of needle wheel carrier one as needle carrier one is being propelled during the firing process;
[e] a body to which said wings and said needle can be embedded or attached;
[f] a body wherein the cross section of said body is rectangular;
[g] a body wherein the cross section of said body is round;
[h] a body wherein the cross section of said body is elliptical;
[k] a needle carrier in which all parts are metal;
[l] a needle carrier in which all parts are plastic except for said needle, and said needle is metal; and
[m] A needle carrier in which the parts are made of a combination of metal and plastic.

14. A reusable lancet device as recited in claim 1 wherein Needle carrier two as recited in claim 1 is comprised of:
[a] a needle made from a cylindrical rod of metal pointed on at least the distal end, and embedded in the body of said needle carrier two such that the needle will remain fixedly-embedded at least during the use of said needle carrier and its needle for lancet purposes;
[b] flexible wings protruding from the body of said needle carrier two at the end opposite the end where said needle is projecting;
[c] jaws at the distal end of said wings, such that said jaws can grasp probe/retractor two whereby said needle carrier two can be propelled or retracted by the action of said probe/retractor two;
[d] jaws which are, as shown on drawings accompanying the present patent application, sculpted in shapes complementary to parts on said probe/retractor two wherein the inclination of said needle carrier two and said probe/retractor two to slip, separate, or otherwise malfunction is reduced;
[e] a body to which said wings and said needle can be embedded or attached;
[f] a body wherein the cross section of said body is rectangular;
[g] a body wherein the cross section of said body is round;
[h] a body wherein the cross section of said body is elliptical;
[k] a needle carrier in which all parts are metal;
[l] a needle carrier in which all parts are plastic except for said needle; and
[m] A needle carrier in which the parts are made of a combination of metal and plastic.

15. A reusable lancet device as recited in claim 1 wherein Needle carrier three as recited in claim 1 is comprised of:
[a] a needle made from a cylindrical rod of metal pointed on at least the distal end, and embedded in the body of said needle carrier three such that the needle will remain fixedly-embedded at least during the use of said needle carrier and its needle for lancet purposes;
[b] flexible wings protruding from the body of said needle carrier three at the end opposite the end where said needle is projecting;
[c] protrusions on said wings which are contained within said needle carrier wheel one until said needle carrier three is retracted, and are then exposed such that reentry of the needle carrier three into the needle-carrier wheel will be prevented because said protrusions will catch on the inner edge of said needle-carrier wheel;
[d] jaws at the distal end of said wings, such that said jaws can grasp probe/retractor three whereby said needle carrier three can be propelled or retracted by the action of said probe/retractor three;
[e] jaws which are, as shown on drawings accompanying the present patent application, sculpted in shapes complementary to parts on said probe/retractor three wherein the inclination of said needle carrier three and said probe/retractor three to slip, separate, or otherwise malfunction is reduced;
[f] a body to which said wings and said needle can be embedded or attached;
[g] a body wherein the cross section of said body is rectangular;
[h] a body wherein the cross section of said body is round;
[k] a body wherein the cross section of said body is elliptical;
[l] a needle carrier in which all parts are metal;
[m] a needle carrier in which all parts are plastic except for said needle; and
[m] A needle carrier in which the parts are made of a combination of metal and plastic.

16. A reusable lancet device as recited in claim 1 wherein Needle carrier four as recited in claim 1 is comprised of:
[a] a needle made from a cylindrical rod of metal pointed on at least the distal end, and embedded in the body of said needle carrier four such that the needle will remain fixedly-embedded at least during the use of said needle carrier and its needle for lancet purposes;
[b] flexible wings protruding from the body of said needle carrier four at the end opposite the end where said needle is projecting;
[c] protrusions on said wings which are contained within said needle carrier wheel one until said needle carrier four is retracted, and are then pressed into pockets inside needle-carrier wheel one, and thereupon said needle carrier four will be prevented from reentry into needle-carrier wheel one.
[d] jaws at the distal end of said wings, such that said jaws can grasp probe/retractor three whereby said needle carrier four can be propelled or retracted by the action of said probe/retractor three;
[e] jaws which are, as shown on drawings accompanying the present patent application, sculpted in shapes complementary to parts on said probe/retractor three wherein the inclination of said needle carrier four and said probe/retractor three to slip, separate, or otherwise malfunction is reduced;
[f] a body to which said wings and said needle can be embedded or attached;
[g] a body wherein the cross section of said body is rectangular;
[h] a body wherein the cross section of said body is round;
[k] a body wherein the cross section of said body is elliptical;
[l] a needle carrier in which all parts are metal;
[m] a needle carrier in which all parts are plastic except for said needle; and
[n] A needle carrier in which the parts are made of a combination of metal and plastic.

17. A reusable lancet device as recited in claim 1 wherein Needle carrier five as recited in claim 1 is comprised of:
[a] a needle made from a cylindrical rod of metal pointed on at least the distal end, and embedded in the body of said needle carrier five such that the needle will remain fixedly-embedded at least during the use of said needle carrier five and its needle for lancet purposes;
[b] a needle-carrier stop-rim made integrally with said needle carrier five, and comprised of a raised ring around the perimeter of the end of the body of said needle carrier five opposite said exposed needle point, such that said needle-carrier stop-rim will prevent said needle carrier from being propelled into the needle-carrier wheel further than the point at which said needle-carrier stop-rim meets the inner edge of said needle-carrier wheel;
[c] a needle-carrier cap-connector made integrally with said needle carrier, and comprised of an appendage to said needle carrier at the end of the body of said needle carrier opposite said needle point, the cross section of said needle-carrier cap-connector being made round, rectangular, or elliptical in shape.
[d] a needle-carrier cap made integrally with the body of needle carrier five, and comprised of a circular disc shaped to receive the grasp of probe/retractor four as recited in claim 1 in such way that the inclination for probe/retractor four and said needle carrier cap to become unexpectedly separated will be reduced, so that the work of said probe/retractor four in propelling and retracting said needle-carrier five will be carried out without failure;
[e] a body to which said needle-carrier stop-rim, said needle-carrier cap-connector and said needle can be embedded or attached;
[f] a body wherein the cross section of said body is round;
[g] a body wherein the cross section of said body is elliptical;
[h] a needle carrier in which all parts are metal; and
[k] a needle carrier in which all parts are plastic except for said needle.

18. A reusable lancet device as recited in claim 1 wherein Needle carrier six, the rimmed and the rimless varieties, as recited in claim 1 is comprised of:
[a] a needle made from a cylindrical rod of metal pointed on at least the distal end, and embedded in the body of said needle carrier six such that the needle will remain fixedly-embedded at least during the use of said needle carrier six and its needle for lancet purposes;
[b] a needle-carrier stop-rim on the rimmed variety made integrally with said needle carrier six, and comprised of a raised ring around the perimeter of the end of said needle carrier six opposite said exposed needle point, such that said needle-carrier stop-rim will prevent said needle carrier from being propelled into the needle-carrier wheel further than the point at which said needle-carrier stop-rim meets the inner edge of said needle-carrier wheel;
[c] a needle-carrier cap-connector made integrally with said needle carrier six, and comprised of an appendage to said needle carrier at the end of the body of said needle carrier opposite said needle point, the cross section of said needle-carrier cap-connector being made round, rectangular, or elliptical in shape;
[d] a needle-carrier cap made integrally with said needle carrier six, and comprised of a knob shaped to receive the grasp of probe/retractor five as recited in claim 1 in such way that the inclination for probe/retractor five and said needle carrier cap to become unexpectedly separated will be reduced, wherein the work of said probe/retractor five in propelling and retracting said needle-carrier six will be carried out without failure;
[e] at least two stop arms projecting from the body of said needle carrier six, and made integrally with said body, wherein said stop arms are hidden within the tunnel in which said needle carrier six is positioned within needle-carrier wheel three until said needle carrier six is retracted after making a skin puncture, at which time the free ends of said stop arms will move away from the body of needle carrier six to the extent that said stop arms can catch on the inner edge of needle-carrier wheel three, wherein said needle carrier six can no longer be fully moved into said tunnel, thus preventing an accidental reuse of said needle carrier six;
[f] a body to which said needle-carrier stop-rim, said needle-carrier cap-connector, said stop arms, and said needle can be embedded or attached;
[g] a body wherein the cross section of said body is round;
[h] a body wherein the cross section of said body is elliptical;
[k] a body wherein the cross section of said body is rectangular;
[l] a needle carrier in which all parts are metal;
[m] a needle carrier in which all parts are plastic except for said needle; and
[n] A needle carrier in which the parts are made of a combination of metal and plastic.

19. A reusable lancet device as recited in claim 1 wherein Probe/retractor one is comprised of:
[a] a cylindrical shaft wherein its distal end is blunted to ease its entry between the wings of needle carrier one as recited in claim 13;
[b] a cylindrical shaft wherein slots are made along the side of said shaft such that said slots are complementary with protrusions on said wings as recited in claim 13, wherein at the time said needle carrier one is impacted by probe/retractor one, said slots and said protrusions will nest together whereby movement of said probe/retractor one can move said needle carrier one and the needle point in needle carrier one to a position whereby said needle point can puncture skin located at the needle aperture recited in claim 2, and, further, will retract said needle carrier one after said puncture may have taken place until said probe/retractor one and said needle carrier one have separated;
[c] a cylindrical shaft such that the end opposite the distal end is able to be fastened into the distal end of hammer one as recited in claim 6 such that said shaft moves in conjunction with said hammer one;
[d] a cylindrical shaft made an integral part of said hammer one, and formed on its distal end;
[e] a probe/retractor as recited in claim 1 wherein the cross section of said shaft is rectangular or elliptical instead of circular
[f] a shaft made of metal;
[g] a shaft made of plastic; and
[h] a shaft made of a combination of metal and plastic.

20. A reusable lancet device as recited in claim 1 wherein Probe/retractor two is comprised of:
[a] a shaft wherein its distal end has a connected cap such that said cap has deformations making it equipped to be securely grasped by the jaws of needle carrier two which is to be made equipped with complementary deformations;
[b] a shaft wherein at the time said needle carrier two is impacted by said probe/retractor two, said jaws of needle carrier two will grasp the cap of probe/retractor two and together probe/retractor two and said needle carrier two and the needle point in needle carrier two will move in conjunction to a position whereby said needle point can puncture skin located at the needle aperture recited in claim 2, and, further, said probe/retractor two will retract said needle carrier two after said puncture may have taken place until said probe/retractor two and said needle carrier two have separated;
[c] a shaft such that the end opposite the distal end is able to be fastened into the distal end of hammer one as recited in claim 6 such that said shaft moves in conjunction with said hammer one;
[d] a cylindrical shaft made an integral part of said hammer one, and formed on its distal end;
[e] a probe/retractor as recited in claim 1 wherein the cross section of said shaft is rectangular, circular, or elliptical in shape;
[f] a shaft made of metal;
[g] a shaft made of plastic; and
[h] a shaft made of a combination of metal and plastic.

21. A reusable lancet device as recited in claim 1 wherein Probe/retractor three is comprised of:
[a] a shaft wherein its distal end has a connected cap such that said cap has deformations making it equipped to be securely grasped by the jaws of either needle carrier three or needle carrier four, wherein said jaws are to have complementary deformations;
[b] a shaft wherein at the time said needle carrier three or needle carrier four are impacted by said probe/retractor three, said jaws of either needle carrier three or needle carrier four will be spread then grasp the cap of probe/retractor three and together probe/retractor three and said needle carriers and the needle point in said needle carriers will move in conjunction to a position whereby said needle point can puncture skin located at the needle aperture recited in claim 2, and, further, said probe/retractor three will retract said needle carrier three or said needle carrier four after said puncture may have taken place until said probe/retractor three and said needle carrier three or said needle carrier four have separated;
[c] a shaft such that the end opposite the distal end is able to be fastened into the distal end of hammer one as recited in claim 6 such that said shaft moves in conjunction with said hammer one;
[d] a shaft made an integral part of said hammer one, and formed on its distal end;
[e] a probe/retractor as recited in claim 1 wherein the cross section of said shaft is rectangular, circular, or elliptical in shape;
[f] a shaft made of metal; and
[g] a shaft made of plastic.

22. A reusable lancet device as recited in claim 1 wherein Probe/retractor four is comprised of:
[a] flexible wings each having a jaw at its distal end, said jaw made with deformations enabling a secure connection with complementary deformations on the cap on needle carrier five as recited in claim 1;
[b] jaws which will be spread or closed when meeting or withdrawing from the stop as recited in claim 10[h], wherein a cap on needle carrier five as recited in claim 17[d] can be grasped by said jaws such that probe/retractor four together with needle carrier five and the needle point in said needle carrier five can move in conjunction to a position whereby said needle point can puncture skin located at the needle aperture recited in claim 2, and, further, said probe/retractor four can retract said needle carrier five after said puncture may have taken place until said probe/retractor four and said needle carrier five have separated;
[c] a body made integrally with said wings and said jaws, wherein said wings and jaws are connected to the distal end of said body;
[d] a body made whereby it can be an integral part of and located at the distal end of hammer two as recited in claim 1;
[e] a body made as a separate part able to be fastened to or in the distal end of said hammer two;
[f] a body made of metal;
[g] a body made of plastic; and
[h] a body made of a combination of metal and plastic.

23. A reusable lancet device as recited in claim 1 wherein Probe/retractor five is comprised of:
[a] flexible wings each having a jaw at its distal end, said jaw made with deformations enabling a secure connection with complementary deformations on the cap on needle carrier six as recited in claim 1;
[b] jaws which will be spread or closed when meeting or withdrawing from the stop as recited in claim 10[h], wherein a cap on needle carrier six as recited in claim 17[d] can be grasped by said jaws such that probe/retractor five together with needle carrier six and the needle point in said needle carrier six can move in conjunction to a position whereby said needle point can puncture skin located at the needle aperture recited in claim 2, and, further, said probe/retractor five can retract said needle carrier six after said puncture may have taken place until said probe/retractor five and said needle carrier six have separated;
[c] a body made integrally with said wings and said jaws, wherein said wings and jaws are connected to the distal end of said body;
[d] a body made whereby it can be an integral part of and located at the distal end of hammer three as recited in claim 1;
[e] a body made as a separate part able to be fastened to or in the distal end of said hammer three;
[f] a body made of metal;
[g] a body made of plastic; and
[h] a body made of a combination of metal and plastic.

24. A reusable lancet device as recited in claim 1 wherein a cog wheel is comprised of:
[a] an irregular bottom surface, circular and inner edges, and a flat top surface or with a raised peripheral bumper rim as a needle-carrier stop;
[b] a group of cogs on the inner edge of said cog wheel such that the movement of the rocker arm recited in claim 1 will rotate said cog wheel and said cog wheel's companion needle-carrier wheel in a way which will accurately position the needle carriers in said needle-carrier wheel for propulsion towards the needle aperture recited in claim 1 and claim 2;
[c] a rubbing rail at the outer periphery and bottom of said cog wheel, said rubbing rail being situated around the periphery of the rubbing rail of wheel carrier two when said cog wheel is in its working location, with said wheel carrier and said cog wheel having a common center;
[d] a cog wheel stop post made integrally with said cog wheel, projecting from the bottom of the cog wheel, and located such that it will meet, and cause said cog wheel to be stopped by, a wheel stop tab located on wheel carrier two as recited in claim 10[f], said stoppage to occur at the time the last unused needle carrier has be used, wherein no used needle carriers on the then-present needle-carrier wheel are likely to be used again for making skin punctures;
[e] at least three pins embedded eccentrically in said cog wheel's upper surface, and projecting such that the cog wheel's companion needle-carrier wheel, having complementary holes through its body, can be properly located atop said cog wheel, and will rotate in conjunction with said cog wheel;
[f] numbering, lettering, signage, and markings made with an indelible dark color, and needed for the proper usage of said cog wheel;
[g] a cog wheel made of metal;
[h] a cog wheel made of plastic;
[k] a cog wheel made of a combination of metal and plastic

25. A reusable lancet device as recited in claim 1 wherein a detent leaf with protrusion is comprised of:
[a] a flat spring detent leaf together with a means of fastening said detent leaf to a detent ramp located on the floor of wheel carrier one as recited in claim 9[k];
[b] a protrusion on said detent leaf such that said protrusion will move into complementary recesses located on the bottom of needle-carrier wheel one as said needle-carrier wheel one is rotated, wherein said protrusion will hold said needle-carrier wheel one in a fixed position until its rotation occurs again;
[c] the fastenings required for fastening said detent leaf to said detent ramp;
[d] a detent leaf and protrusion made of metal;
[e] a detent leaf and protrusion made of plastic; and
[f] a detent leaf and protrusion made of a combination of metal and plastic.

26. A reusable lancet device as recited in claim 1 wherein a cog wheel detent is comprised of:
[a] a bevel-ended, spring-loaded finger used to engage a cog face of the cog wheel recited in claim 1 for the purpose of obstructing said cog wheel together with its coupled needle-carrier wheel two or three such that said cog wheel and its coupled needle-carrier wheel two or three are restricted from being rotated in an incorrect direction;
[b] a bevel-ended, spring-loaded finger used to engage a cog face in said cog wheel for the purpose of keeping said cog wheel and its coupled needle-carrier wheel two or three in a correct position such that an unused needle carrier, having been moved into the correct position for its propulsion, will remain in said correct position until after it has been propelled in the direction of the needle aperture recited in claim 2;
[c] a body such that said bevel-ended, spring-loaded finger is cased wherein it can remain assembled and can be fastened securely atop the detent-dog pedestal recited in claim 10[l];
[d] a retaining pin through said spring-loaded finger to retain said finger without impeding its required movements;
[e] parts required for fastenings, washers, and springs;
[f] said cog wheel detent as recited in claim 1 in which said cog wheel detent is made of metal;
[g] said cog wheel detent as recited in claim 1 in which said cog wheel detent is made of plastic; and
[h] said cog wheel detent as recited in claim 1 in which said cog wheel detent is made of a combination of metal and plastic.

27. A reusable lancet device as recited in claim 1 wherein numbering, lettering, and markings are comprised of:
[a] dark, indelible, marks on any of the parts of said lancet device such that said marks will provide unambiguous information about the use of said lancet device for the user of said lancet device;
[b] markings comprising instructions about the use of said lancet device for the user of said lancet device; and
[c] markings comprising information for the user of said lancet device.

28. A reusable lancet device as recited in claim 1 wherein fastenings, pins, springs, washers, screws, bolts, and nuts are comprised of:
[a] metal suitable for the use to which said fastenings, pins, springs, washers, screws, bolts, and nuts will be consigned in said lancet device;
[b] plastic suitable for the use to which said fastenings, pins, springs, washers, screws, bolts, and nuts will be consigned in said lancet device; and
[c] a combination of metal and plastic suitable for the use to which said fastenings, pins, springs, washers, screws, bolts, and nuts will be consigned in said lancet device.
